(19) United States
(12) Patent Application Publication
(10) Pub. No.: US 2024/0252553 A1
(43) Pub. Date: Aug. 1, 2024

(54) ENGINEERED S. TYPHIMURIUM AND USES THEREOF

(71) Applicant: VIRGINIA TECH INTELLECTUAL PROPERTIES, INC., Blacksburg, VA (US)

(72) Inventors: Bahareh BEHKAM, Blacksburg, VA (US); Eric LEAMAN, Blacksburg, VA (US)

(21) Appl. No.: 18/578,856

(22) PCT Filed: Jul. 12, 2022
(86) PCT No.: PCT/US22/36796

§ 371 Date: Jan. 12, 2024

Related U.S. Application Data
(60) Provisional application No. 63/220,775, filed on Jul. 12, 2021.

(51) Int. Cl. A61K 35/74; A61K 45/06; A61P 35/00; C12N 9/52; C12N 15/74

(52) U.S. Cl. CPC A61K 35/74; A61K 45/06; A61P 35/00; C12N 9/52; C12N 15/74; C12Y 304/24003

(57) ABSTRACT

Cancer is a significant cause of mortality and morbidity worldwide. One of the principal impediments to the broad success of conventional chemotherapy is poor delivery to and transport within the tumor microenvironment (TME), caused by irregular and leaky vasculature, the lack of functional lymphatics, and underscored by the overproduction of extracellular matrix (ECM) proteins such as collagen. Described herein are engineered Salmonella Typhimurium (S. Typhimurium) bacteria and uses thereof. In some embodiments, the engineered S. Typhimurium bacteria comprises an exogenous collagenase. Also described in exemplary embodiments herein methods of using the engineered S. Typhimurium bacteria, such as part of a,cancer therapy.

### CROSS-REFERENCE TO RELATED APPLICATIONS

This application claims the benefit of and priority to co-pending U.S. Provisional Patent Application No. 63/220,775, filed on Jul. 12, 2021, entitled “ENGINEERED S. TYPHIMURIUM AND USES THEREOF,” the contents of which is incorporated by reference herein in its entirety.

### STATEMENT REGARDING FEDERALLY SPONSORED RESEARCH

This invention was made with government support under Grant No.(s) CBET-1454226 awarded by the National Science Foundation. The government has certain rights in the invention.

### TECHNICAL FIELD

The subject matter disclosed herein is generally directed to engineered Salmonella Typhimurium and uses thereof.

### BACKGROUND

Cancer is a significant cause of mortality and morbidity worldwide. One of the principal impediments to the broad success of conventional chemotherapy is poor delivery to and transport within the tumor microenvironment (TME), caused by irregular and leaky vasculature, the lack of functional lymphatics, and underscored by the overproduction of extracellular matrix (ECM) proteins such as collagen. Coupled with limited specificity, the high chemotherapeutic doses needed to effectively treat tumors often lead to unacceptable levels of damage to healthy tissues. Bacteria-based cancer therapy (BBCT) is an innovative alternative. However, current BBCT approaches have failed to achieve clinical success due primarily to a lack of sufficient tumor colonization. As such there exists a need for improved cancer therapies, including improved BBCTs.

Citation or identification of any document in this application is not an admission that such a document is available as prior art to the present invention.

### SUMMARY

Described in certain example embodiments herein are engineered Salmonella Typhimurium (S. Typhimurium) bacterium, population thereof, and/or progeny thereof, the engineered S. Typhimurium bacterium comprising: an exogenous collagenase encoding polynucleotide, polypeptide product thereof, or both, wherein the engineered S. Typhimurium strain is an S. Typhimurium14028 or S. Typhimurium VNP20009.

In certain example embodiments, the collagenase encoding polynucleotide is or encodes a collagenase or functional domain thereof as set forth in Table 1.

In certain example embodiments, the exogenous collagenase gene is a metalloproteinase gene.

In certain example embodiments, the collagenase gene is prtV from Vibrio parahaemolyticus EB101, a homologue thereof, an orthologue thereof, or a paralogue thereof.

In certain example embodiments, the exogenous collagenase encoding polynucleotide is present on a plasmid, cosmid, or artificial chromosome.

In certain example embodiments, the exogenous collagenase encoding polynucleotide is operably coupled to one or more regulatory elements, optionally wherein the one or more regulatory elements is or comprises a promoter, wherein the promoter is a constitutive promoter, inducible promoter, tissue or tumor specific promoter, or any permissible combination thereof.

In certain example embodiments, the exogenous collagenase encoding polynucleotide is constitutively expressed, is inducibly expressed, or is selectively expressed by the engineered bacterium.

In certain example embodiments, the engineered bacterium, population thereof, and/or progeny thereof has increased tumor or tumor microenvironment penetration, increased tumor microenvironment retention, increased tumor colonization, or any combination thereof as compared to a parent S. Typhimurium, optionally of the strain S. Typhimurium 14028 or strain S. Typhimurium VNP20009.

In certain example embodiments, the engineered bacterium, population thereof, and/or progeny thereof is capable of degrading a collagen matrix.

In certain example embodiments, the engineered bacterium, population thereof, and/or progeny thereof is capable of producing and/or secreting a collagenase polypeptide and/or functional domain thereof.

In certain example embodiments, the engineered bacterium, population thereof and/or progeny thereof further comprises a second active agent, a cargo, or both, wherein the second active agent, cargo, or both is/are coupled to, integrated with, contained within, or otherwise associated with the engineered bacterium, population thereof, and/or progeny thereof.

In certain example embodiments, collagen matrix penetration, tumor microenvironment penetration, and/or extracellular matrix penetration is increased 10-1,000 percent or more as compared to a S. Typhimurium parent bacterium, optionally of the strain S. Typhimurium 14028 or strain S. Typhimurium VNP20009.

Described in certain example embodiments are pharmaceutical formulations comprising: an engineered bacterium, population thereof, and/or progeny thereof as described herein; and a pharmaceutically acceptable carrier. In certain example embodiments, the pharmaceutical formulation further comprises one or more secondary active agents. In certain example embodiments, the one or more secondary active agents is/are or comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, a genetic modifying agent, a vaccine, or a combination thereof.

Described in certain example embodiments herein are methods of (a) treating and/or preventing a disease or a symptom thereof in a subject. (b) modifying a cell, tissue, organ, and/or tumor microenvironment of a subject, (c) modifying an extracellular matrix or component thereof optionally of a subject, (d) modifying a collagen matrix optionally of a subject; or (e) any combination of (a)-(d) the method comprising: administering an engineered bacterium, population thereof, and/or progeny thereof as described herein or a pharmaceutical formulation thereof to the subject, extracellular matrix or component thereof, collagen matrix, or combination thereof.

In certain example embodiments, the disease is a cancer. In certain example embodiments, the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis fungoide, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.

In certain example embodiments, the method further comprises administering one or more secondary active agents to the subject. In certain example embodiments, the one or more secondary active agents comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, a genetic modifying agent, a vaccine, or any combination thereof.

In certain example embodiments, the engineered bacterium, population thereof, and/or progeny thereof as described herein, or the pharmaceutical formulation thereof is effective to treat a disease in the subject in need thereof.

Described in certain example embodiments herein are kits for treating and/or preventing a disease in a subject in need thereof comprising: an engineered bacterium, population thereof, and/or progeny thereof described herein or a pharmaceutical formulation thereof, optionally one or more secondary active agents, and/or optionally one or more delivery reagents and/or devices, one or more storage reagents and/or devices, one or more culture reagents and/or devices, or any combination thereof; and instructions in a tangible medium expression directing a user to administer the engineered bacterium, population thereof, and/or progeny thereof as described herein or a pharmaceutical formulation thereof 5, and optionally the one or more secondary active agents to the subject in need thereof.

In certain example embodiments, the subject in need thereof has a cancer. In certain example embodiments, the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis a fungoid, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Cloning of prtV into a Robust Plasmid Construct for Expression in S. Typhimurium. (FIG. 1A) A map of the plasmid constructed for this work and (FIG. 1B) screening of transformed colonies of E. coli harboring the constructed plasmid after cloning of each of the two fragments of prtV (2048 bp and 1110 bp represent successful cloning of the full gene and the first gene fragment, respectively). The constructed plasmid is based on a medium copy number vector for arabinose-inducible expression, but we replaced the pBAD promoter with the synthetic constitutive promoter BBa_J23100. Downstream of prtV, we inserted mRFP1 driven independently by the E. coli lac promoter for constitutive expression.

FIG. 2A-2C—Microfluidic Experiments. (FIG. 2A) Schematic of the microfluidic device used to quantitate the transport of non-motile S. Typhimurium VNP20009prtV and its parental counterpart. (FIG. 2B) a composite bright-field and fluorescent micrograph showing the distribution of S. Typhimurium VNP20009prtV in the device after about 20 hr, (FIG. 2C) fluorescent micrographs and heatmaps showing the relative bacterial colonization of the collagen gel in space and time.

FIG. 3A-3E—Gelatin-based Assays to Confirm Expression, Secretion, and Activity of PrtV. FIG. 3A-FIG. 3C show V. parahaemolyticus (positive control), S. Typhimurium 14028 and VNP20009 (negative controls), and S. Typhimurium 14028prtV and VNP20009prtV, respectively, colonies on agar plates supplemented with 2% gelatin and stained with Coomassie Brilliant Blue. FIG. 3D shows localized growth of the VNP20009prtV strain in semi-solid nutrient medium supplemented with 10% gelatin several days after inoculation. FIG. 3E shows test tubes originally containing this same semi-solid nutrient medium several weeks after inoculation with VNP20009prtV (left) and wild-type VNP20009 (right). The medium was liquefied in the prtV case but remains a semi-solid in the control case, with the VNP20009 control localized near the gel-air interface where it was initially seeded.

FIG. 4A-4B—Measurements of Proteolytic Activity against Collagen Type I by Engineered VNP20009. (FIG. 4A) DQ collagen type I probe fluorescence and (FIG. 4B) RFP fluorescence vs. time for prtV and control strains encapsulated in 7.1 mg/mL collagen type I. The amount of collagen degraded is proportional to the fluorescent intensity measurements shown in (FIG. 4A), while the bacterial concentration is indicated by data shown in (FIG. 4B). Note that control strains in these experiments expressed the BioBrick part BBa_J04450 in pSB1C3 (high copy number) rather than the plasmid constructed in this work for control experiments (See also Methods, Working Example 1).

FIG. 5A-5E—Quantitation of Transport of Non-motile S. Typhimurium VNP20009prtV in Collagen Type I. (FIG. 5A) Representative fluorescence images of S. Typhimurium VNP20009 (control) and S. Typhimurium VNP20009prtV in the central collagen barriers of microfluidic devices. (FIG. 5B) and (FIG. 5C) the penetration distance and penetration rate, respectively, of each strain as functions of time, (FIG. 5D) and (FIG. 5E) the average penetration and penetration rates, respectively, as functions of time for n_{control}=11 and n_{prtV}=16 experimental replicates.

FIG. 6—An exemplary scheme for engineering a collagenase secreting Salmonella.

FIG. 7—Dye-quenched Collagen Type I assay results.

FIG. 8—Gelatin-based Assays to Confirm Expression, Secretion, and Activity of mmPG.

FIG. 9—Microfluidic setup of advective transport of non-motile bacteria under oscillatory interstitial flow of mmPG expressing S. Typhimrurim VNP2009mmpG.

FIG. 10—Swim plate assay of motile bacteria transport.

FIG. 11—Microfluidic experimental results from control and engineered VNP2009mmpG bacteria.

FIG. 12—Graphs showing penetration rate of control and engineered S. Typhimrurim VNP2009mmpG in collagen type I.

FIG. 13—Graphs showing penetration distance of control and engineered S. Typhimrurim VNP2009mmpG in collagen type I.

FIG. 14—Doubling time of control and engineered S. Typhimrurim VNP2009mmp.

FIG. 15—Diffusion rate of control and engineered S. Typhimrurim VNP2009mmp.

FIG. 16A-16F—Genetic tuning of motile bacteria. (FIG. 16A) Genetic tuning with synthetic biology design of an RBS library. (FIG. 16B) Results of a dye-quenched collagen Type I assay. (FIG. 16C and FIG. 16E) Graph demonstrating doubling time of engineered bacteria and associated relative bacterial concentrations. (FIG. 16D and FIG. 16F) Graph demonstrating swimming speed of engineered bacteria and associated relative bacterial concentrations.

FIG. 17—Microscopic image showing bacteria swimming in about 5 mg/ml collagen.

FIG. 18—Fluorescent microscopic images showing bacteria infiltration into pancreatic tumor organoids.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where “x” and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis): Molecular Cloning: A Laboratory Manual, 4^{th} edition (2012) (Green and Sambrook): Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.): Animal Cell Culture (1987) (R. I. Freshney, ed.); Benjamin Lewin, Genes IX, published by Jones and Bartlett, 2008 (ISBN 0763752223): Kendrew et al. (eds.), The Encyclopedia of Molecular Biology, published by Blackwell Science Ltd., 1994 (ISBN 0632021829): Robert A. Meyers (ed.), Molecular Biology and Biotechnology: a Comprehensive Desk Reference, published by VCH Publishers, Inc., 1995 (ISBN 9780471185710): Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson: Brown et al., Chemistry, The Central Science 14^{th} ed. (2017), published by Pearson, Clayden et al., Organic Chemistry, 2^{nd} ed. 2012, published by Oxford University Press: Carey and Sunberg, Advanced Organic Chemistry, Part A: Structure and Mechanisms, 5^{th} ed. 2008, published by Springer: Carey and Sunberg, Advanced Organic Chemistry, Part B: Reactions and Synthesis, 5^{th} ed. 2010, published by Springer, and Vollhardt and Schore, Organic Chemistry, Structure and Function: 8^{th} ed. (2018) published by W. H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill: Isik et la., Genetic Data Analysis for Plant and Animal Breeding. First ed. 2017. published by Springer International Publishing AG: Green, E. L. Genetics and Probability in Animal Breeding Experiments. 2014, published by Palgrave: Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press: Fasso, D. Classification of Genetic Variance in Animals. First Ed. 2015, published by Callisto Reference: Megahed, M. Handbook of Animal Breeding and Genetics, 2013, published by Omniscriptum Gmbh & Co. Kg., LAP Lambert Academic Publishing: Reece. Analysis of Genes and Genomes. 2004, published by John Wiley & Sons. Inc: Deonier et al., Computational Genome Analysis. 5^{th} Ed. 2005, published by Springer-Verlag, New York: Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994: Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides or polynucleotides of the present disclosure, unless stated otherwise.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

The term “modification causing said increased expression” and the like refers to a modification in a gene which affects the expression level of that or another gene such that expression of that or another gene is increased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used interchangeably herein, “operatively linked” and “operably linked” in the context of recombinant or engineered polynucleotide molecules (e.g., DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “plasmid” refers to a non-chromosomal double-stranded DNA sequence including an intact “replicon” such that the plasmid is replicated in a host cell.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “radiation sensitizer” refers to agents that can selectively enhance the cell killing from irradiation in a desired cell population, such as tumor cells, while exhibiting no single agent toxicity on tumor or normal cells.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

The term “modification causing said reduced expression” refers to a modification in a gene which affects the expression level of that or another gene such that the expression level of that or another gene is reduced or decreased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein covers any treatment of a cancer, in a subject, particularly a human or non-human animal, and can include any one or more of the following: (a) preventing the disease from occurring in a subject which may be predisposed to the disease but has not yet been diagnosed as having it: (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein “tumour” or “tumour tissue” refer to an abnormal mass of tissue resulting from excessive cell division. A tumour or tumour tissue comprises “tumour cells” which are neoplastic cells with abnormal growth properties and no useful bodily function. Tumours, tumour tissue and tumour cells may be benign, pre-malignant or malignant, or may represent a lesion without any cancerous potential. A tumour or tumour tissue may also comprise “tumour-associated non-tumour cells”, e.g., vascular cells which form blood vessels to supply the tumour or tumour tissue. Non-tumour cells may be induced to replicate and develop by tumour cells, for example, the induction of angiogenesis in a tumour or tumour tissue.

As used herein, the term “tumor microenvironment” (TME) refers to is the cellular environment in which the tumor exists, including surrounding blood vessels, immune cells, cancer associated fibroblasts (CAFs), bone marrow-derived inflammatory cells, lymphocytes, signaling molecules and the extracellular matrix (ECM).

As used herein, the term “vector” or is used in reference to a vehicle used to introduce an exogenous nucleic acid sequence into a cell. A vector may include a DNA molecule, linear or circular (e.g., plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, the terms “weight percent.” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “exogenous” refers to a molecule, such as a polynucleotide, that is not native (or endogenous) to the host organism into which it is introduced.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Cancer is a significant cause of mortality and morbidity worldwide. One of the principal impediments to the broad success of conventional chemotherapy is poor delivery to and transport within the tumor microenvironment (TME), caused by irregular and leaky vasculature, the lack of functional lymphatics, and underscored by the overproduction of extracellular matrix (ECM) proteins such as collagen. Coupled with limited specificity, the high chemotherapeutic doses needed to effectively treat tumors often lead to unacceptable levels of damage to healthy tissues. Bacteria-based cancer therapy (BBCT) is an innovative alternative. However, current BBCT approaches have failed to achieve clinical success due primarily to a lack of sufficient tumor colonization. As such there exists a need for improved cancer therapies, including improved BBCTs.

That said described in certain example embodiments herein is a modified strain of the attenuated tumor-targeting bacteria Salmonella Typhimurium VNP20009 which constitutively expresses and secretes a bacterial collagenase. As demonstrated in the Working Examples, the exemplary modified strain, hereafter referred to as S. Typhimurium VNP20009prtV, was constructed by cloning a recombinant bacterial metalloproteinase into a plasmid construct under control of a constitutive promoter. Evidence of expression, secretion, and activity of the enzyme was confirmed using gelatin plate assays and dye-quenched collagen type I. The Working Examples herein at least demonstrate a significant enhancement of penetration and colonization by S. Typhimurium VNP20009prtV relative to controls in collagen gel and in tumor organoids. Described in certain example embodiments is a method of using the engineered bacterium and progeny thereof described herein as a treatment, such as a tumor treatment modality, that (i) can have improved transport and retention characteristics in tumors relative to its parental counterpart, and (ii) can improve the transport of macromolecular substances in the tumor microenvironment.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered S. Typhimurium

Described in certain example embodiments herein are engineered Salmonella Typhimurium (S. Typhimurium) bacterium, population thereof, and/or progeny thereof, the engineered S. Typhimurium bacterium comprising: an exogenous collagenase encoding polynucleotide, polypeptide product thereof, or both, wherein the engineered S. Typhimurium strain is an S. Typhimurium 14028 or S. Typhimurium VNP20009.

In certain example embodiments, the collagenase encoding polynucleotide is or encodes a collagenase or functional domain thereof as set forth in Table 1. In some embodiments, the functional domain is capable of collagenase activity. In some embodiments, the exogenous collagenase encoding polynucleotide is an exogenous collagenase gene or portion thereof encoding a functional domain thereof. In certain example embodiments, the exogenous collagenase encoding polynucleotide is an exogenous metalloproteinase encoding polynucleotide. In some embodiments, the exogenous metalloproteinase encoding polynucleotide is an exogenous metalloproteinase gene or portion thereof encoding a functional domain thereof.

In some embodiments, the collagenase encoding polynucleotide is or encodes a collagenase or functional domain thereof that is 50-100 percent identical to a polynucleotide or polypeptide set forth in Table 1. In some embodiments, the collagenase encoding polynucleotide is or encodes a collagenase or functional domain thereof that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to/or 100% identical to a polynucleotide or polypeptide set forth in Table 1.

In certain example embodiments, the collagenase gene is prtV from Vibrio parahaemolyticus EB101, a homologue thereof, an orthologue thereof, or a paralogue thereof.

In certain example embodiments, the exogenous collagenase encoding polynucleotide is present on a plasmid, cosmid, or artificial chromosome.

In certain example embodiments, the exogenous collagenase encoding polynucleotide is operably coupled to one or more regulatory elements, optionally wherein the one or more regulatory elements is or comprises a promoter, wherein the promoter is a constitutive promoter, inducible promoter, tissue or tumor specific promoter, or any permissible combination thereof.

In certain example embodiments, the exogenous collagenase encoding polynucleotide is constitutively expressed, is inducibly expressed, or is selectively expressed by the engineered bacterium.

In certain example embodiments, the engineered bacterium, population thereof, and/or progeny thereof has increased tumor or tumor microenvironment penetration, increased tumor microenvironment retention, increased tumor colonization, or any combination thereof as compared to a parent S. Typhimurium, optionally of the strain S. Typhimurium 14028 or strain S. Typhimurium VNP20009.

In certain example embodiments, the engineered bacterium, population thereof, and/or progeny thereof is capable of degrading a collagen matrix. In some embodiments, the engineered bacterium has a faster rate of collagen matrix degradation as compared to a parent strain, wild-type strain, or non-engineered strain. In some embodiments, the rate of collagen matrix degradation is increased by 1 to 1000 fold or more as compared to a parent strain, wild-type strain, or non-engineered strain. In some embodiments, the rate of collagen matrix degradation is increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, to/or 1000 fold or more.

In certain example embodiments, the engineered bacterium, population thereof, and/or progeny thereof is capable of producing and/or secreting a collagenase polypeptide and/or functional domain thereof. Without being bound by theory, a secreted collagenase polypeptide, which is generated by expression of the exogenous collagenase encoding polynucleotide can degrade a collagen matrix.

In some embodiments, the engineered bacterium, population thereof, and/or progeny thereof has increased collagen matrix penetration, tumor microenvironment penetration, and/or extracellular matrix penetration as compared to a parent strain, wild-type strain, and/or non-engineered strain. In certain example embodiments, collagen matrix penetration, tumor microenvironment penetration, and/or extracellular matrix penetration is increased 10-1,000 percent or more as compared to a S. Typhimurium parent bacterium, optionally of the strain S. Typhimurium 14028 or strain S. Typhimurium VNP20009, wild-type bacterium, and/or non-engineered bacterium. In certain example embodiments, collagen matrix penetration, tumor microenvironment penetration, and/or extracellular matrix penetration is increased 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000, 5010, 5020, 5030, 5040, 5050, 5060, 5070, 5080, 5090, 5100, 5110, 5120, 5130, 5140, 5150, 5160, 5170, 5180, 5190, 5200, 5210, 5220, 5230, 5240, 5250, 5260, 5270, 5280, 5290, 5300, 5310, 5320, 5330, 5340, 5350, 5360, 5370, 5380, 5390, 5400, 5410, 5420, 5430, 5440, 5450, 5460, 5470, 5480, 5490, 5500, 5510, 5520, 5530, 5540, 5550, 5560, 5570, 5580, 5590, 5600, 5610, 5620, 5630, 5640, 5650, 5660, 5670, 5680, 5690, 5700, 5710, 5720, 5730, 5740, 5750, 5760, 5770, 5780, 5790, 5800, 5810, 5820, 5830, 5840, 5850, 5860, 5870, 5880, 5890, 5900, 5910, 5920, 5930, 5940, 5950, 5960, 5970, 5980, 5990, 6000, 6010, 6020, 6030, 6040, 6050, 6060, 6070, 6080, 6090, 6100, 6110, 6120, 6130, 6140, 6150, 6160, 6170, 6180, 6190, 6200, 6210, 6220, 6230, 6240, 6250, 6260, 6270, 6280, 6290, 6300, 6310, 6320, 6330, 6340, 6350, 6360, 6370, 6380, 6390, 6400, 6410, 6420, 6430, 6440, 6450, 6460, 6470, 6480, 6490, 6500, 6510, 6520, 6530, 6540, 6550, 6560, 6570, 6580, 6590, 6600, 6610, 6620, 6630, 6640, 6650, 6660, 6670, 6680, 6690, 6700, 6710, 6720, 6730, 6740, 6750, 6760, 6770, 6780, 6790, 6800, 6810, 6820, 6830, 6840, 6850, 6860, 6870, 6880, 6890, 6900, 6910, 6920, 6930, 6940, 6950, 6960, 6970, 6980, 6990, 7000, 7010, 7020, 7030, 7040, 7050, 7060, 7070, 7080, 7090, 7100, 7110, 7120, 7130, 7140, 7150, 7160, 7170, 7180, 7190, 7200, 7210, 7220, 7230, 7240, 7250, 7260, 7270, 7280, 7290, 7300, 7310, 7320, 7330, 7340, 7350, 7360, 7370, 7380, 7390, 7400, 7410, 7420, 7430, 7440, 7450, 7460, 7470, 7480, 7490, 7500, 7510, 7520, 7530, 7540, 7550, 7560, 7570, 7580, 7590, 7600, 7610, 7620, 7630, 7640, 7650, 7660, 7670, 7680, 7690, 7700, 7710, 7720, 7730, 7740, 7750, 7760, 7770, 7780, 7790, 7800, 7810, 7820, 7830, 7840, 7850, 7860, 7870, 7880, 7890, 7900, 7910, 7920, 7930, 7940, 7950, 7960, 7970, 7980, 7990, 8000, 8010, 8020, 8030, 8040, 8050, 8060, 8070, 8080, 8090, 8100, 8110, 8120, 8130, 8140, 8150, 8160, 8170, 8180, 8190, 8200, 8210, 8220, 8230, 8240, 8250, 8260, 8270, 8280, 8290, 8300, 8310, 8320, 8330, 8340, 8350, 8360, 8370, 8380, 8390, 8400, 8410, 8420, 8430, 8440, 8450, 8460, 8470, 8480, 8490, 8500, 8510, 8520, 8530, 8540, 8550, 8560, 8570, 8580, 8590, 8600, 8610, 8620, 8630, 8640, 8650, 8660, 8670, 8680, 8690, 8700, 8710, 8720, 8730, 8740, 8750, 8760, 8770, 8780, 8790, 8800, 8810, 8820, 8830, 8840, 8850, 8860, 8870, 8880, 8890, 8900, 8910, 8920, 8930, 8940, 8950, 8960, 8970, 8980, 8990, 9000, 9010, 9020, 9030, 9040, 9050, 9060, 9070, 9080, 9090, 9100, 9110, 9120, 9130, 9140, 9150, 9160, 9170, 9180, 9190, 9200, 9210, 9220, 9230, 9240, 9250, 9260, 9270, 9280, 9290, 9300, 9310, 9320, 9330, 9340, 9350, 9360, 9370, 9380, 9390, 9400, 9410, 9420, 9430, 9440, 9450, 9460, 9470, 9480, 9490, 9500, 9510, 9520, 9530, 9540, 9550, 9560, 9570, 9580, 9590, 9600, 9610, 9620, 9630, 9640, 9650, 9660, 9670, 9680, 9690, 9700, 9710, 9720, 9730, 9740, 9750, 9760, 9770, 9780, 9790, 9800, 9810, 9820, 9830, 9840, 9850, 9860, 9870, 9880, 9890, 9900, 9910, 9920, 9930, 9940, 9950, 9960, 9970, 9980, 9990, to/or 10000 percent or more as compared to a S. Typhimurium parent bacterium, optionally of the strain S. Typhimurium 14028 or strain S. Typhimurium VNP20009, wild-type bacterium, and/or non-engineered bacterium.

In certain example embodiments, the engineered bacterium, population thereof and/or progeny thereof further comprises a second active agent, a cargo, or both, wherein the second active agent, cargo, or both is/are coupled to, integrated with, contained within, or otherwise associated with the engineered bacterium, population thereof, and/or progeny thereof. Exemplary secondary agents and/or cargos include, but are not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, genetic modifying agents and any combination thereof. Without being bound by theory, the engineered cells can be further engineered to carry a secondary agent and/or cargo that can be delivered to a target cell, such as a cancer cell.

The engineered bacterium can be cultured, stored, expanded, and/or otherwise propagated using routine cell culture and storage techniques, which will be appreciated by those of ordinary skill in the art.

### Additional Exemplary Exogenous Collagenases

TABLE 1
Exemplary Exogenous Collagenases

1. Human collagenase gene, 5′ end (698 bp linear DNA, M16567.1 GI: 180668).
2. Bacillus subtilis extracellular metalloprotease (mpr) gene, complete cds (998 bp linear DNA, L10505.1
GI: 143209).
3. Cytophaga sp. DNA for collagenase, complete cds (5,653 bp linear DNA, D50600.1 GI: 849045).
4. Streptomyces lividans metalloprotease (prt) gene, complete cds (1,899 bp linear DNA,. M89476.1
GI: 153411).
5. Streptomyces cacaoi extracellular metalloprotease (npr) gene (1,837 bp linear DNA, M37055.1
GI: 153374).
6. Vibrio vulnificus vvp gene for metalloprotease, type strain CECT 4602T (3,045 bp linear DNA,
AM492792.1 GI: 169642808).
7. Bacteroides fragilis bft-2 gene for metalloprotease, complete cds, (1,546 bp linear DNA,. AB026626.1
GI: 4757386).
8. Bacteroides fragilis bft-1 gene for metalloprotease, complete cds (1,546 bp linear DNA, AB026625.1
GI: 4757384).
9. Bacteroides fragilis bft-3 gene for metalloprotease, complete cds (1,547 bp linear DNA, AB026624.1
GI: 4757372).
10. Pseudomonas fluorescens gene for metalloprotease, complete cds (1,431 bp linear DNA, AB013895.1
GI: 3135219).
11. V.alginolyticus gene for collagenase (3,927 bp linear DNA, X62635.1 GI: 48325).
12. Aspergillus fumigatus metalloprotease exons 1-5, complete cds (2,692 bp linear DNA,. L29566.1
GI: 461376).
13. Renibacterium salmoninarum hly gene for metalloprotease (1,870 bp linear DNA,. X76499.2
GI: 18250640).
14. Porphyromonas gingivalis DNA for collagenase, complete cds (1,306 bp linear DNA, AB006973.1
GI: 2505966).
15. Vibrio aestuarianus metalloprotease precursor (VAM) gene, complete cds (1,852 bp linear DNA,
AY605667.1 GI: 78125891).
16. Vibrio vulnificus metalloprotease gene, complete cds (2,660 bp linear DNA, U50548.1 GI: 2114338)
17. Serratia sp. KCK metalloprotease precursor, gene, complete cds (1,936 bp linear DNA, EF191201.1
GI: 124518423)
18. Clostridium perfringens colA gene for collagenase, complete cds (4,141 bp linear DNA, D13791.1
GI: 440850)
19. L. monocytogenes mpl gene for metalloprotease (1,952 bp linear DNA, X54619.1 GI: 44114).
20. Clostridium histolyticum colH gene for collagenase, complete cds (3,500 bp linear DNA, D29981.1
GI: 563954).
21. Serratia marcescens metalloprotease gene (3,871 bp linear DNA, X55521.1 GI: 47238).
22. Vibrio sp. Pr21 gene for metalloprotease, complete cds (1,887 bp linear DNA, AB734038.2
GI: 1818460859).
23. Serratia plymuthica strain UBCF_13 metalloprotease gene, complete cds (1,059 bp linear DNA,
MK524938.1 GI: 1733436778).
24. Serratia plymuthica strain UBCF_01 metalloprotease gene, complete cds (1,059 bp linear DNA,
MK524937.1 GI: 1733436776).
25. Serratia plymuthica strain UBCR_12 metalloprotease gene, complete cds (1,059 bp linear DNA,
MK524936.1 GI: 1733436774).
26. Aeromonas piscicola strain AH-3 collagenase (colAh) gene, complete cds (2,748 bp linear DNA,
JQ639076.1 GI: 399762992).
27. Flavobacterium psychrophilum gene for collagenase, complete cds, isolate: WA-2 (2,886 bp linear DNA,
AB921327.1 GI: 765715998).
28. Flavobacterium psychrophilum gene for collagenase, complete cds, isolate: WA-1 (2,883 bp linear DNA,
AB921326.1 GI: 765715995).
29. Flavobacterium psychrophilum fppl gene for metalloprotease, complete cds, isolate: WB-1 (3,417 bp
linear DNA, AB921322.1 GI: 765715977).
30. Flavobacterium psychrophilum fppl gene for metalloprotease, complete cds, isolate: WA-2 (3,420 bp
linear DNA, AB921321.1 GI: 765715972).
31. Aeromonas hydrophila strain 450 metalloprotease gene, complete cds (1,773 bp linear DNA, JQ613372.1
GI: 386785840).
32. Aeromonas hydrophila strain 438 metalloprotease gene, complete cds (1,773 bp linear DNA, JQ613371.1
GI: 386785838).
33. Aeromonas hydrophila strain 434 metalloprotease gene, complete cds (1,773 bp linear DNA, JQ613370.1
GI: 386785836).
34. Aeromonas hydrophila strain 431 metalloprotease gene, complete cds (1,773 bp linear DNA, JQ613369.1
GI: 386785834).
35. Aeromonas hydrophila strain 421 metalloprotease gene, complete cds (1,773 bp linear DNA, JQ613368.1
GI: 386785832).
36. Aeromonas hydrophila strain 433 metalloprotease gene, complete cds (1,770 bp linear DNA, JQ613367.1
GI: 386785830).
37. Aeromonas hydrophila strain 428 metalloprotease gene, complete cds (1,770 bp linear DNA, JQ613366.1
GI: 386785828).
38. Aeromonas hydrophila strain 405 metalloprotease gene, complete cds (1,770 bp linear DNA, JQ613365.1
GI: 386785826).
39. Aeromonas hydrophila strain 406 metalloprotease gene, complete cds (1,770 bp linear DNA, JQ613364.1
GI: 386785824).
40. Aeromonas hydrophila strain 412 metalloprotease gene, complete cds (1,770 bp linear DNA, JQ613363.1
GI: 386785822).
41. Aeromonas hydrophila strain 451 metalloprotease gene, complete cds (1,770 bp linear DNA, JQ613362.1
GI: 386785820).
42. Aeromonas hydrophila strain 453 metalloprotease gene, complete cds (1,770 bp linear DNA, JQ613361.1
GI: 386785818).
43. Serratia sp. F1390 metalloprotease gene, complete cds (1,515 bp linear DNA, KF372856.1
GI: 541129133).
44. Grimontia hollisae gene for collagenase, complete cds, strain: 1706B (2,304 bp linear DNA, AB600550.1
GI: 336088230).
45. Vibrio parahaemolyticus collagenase gene, complete cds (2,445 bp linear DNA, AF326572.1
GI: 12584927).
46. Listonella anguillarum strain M93Sm metalloprotease (empA) gene, complete cds (2,220 bp linear DNA,
AY428808.1 GI: 40557597).
47. Pseudomonas fluorescens metalloprotease (aprX) gene, complete cds (1,941 bp linear DNA, AF216700.1
GI: 8895496).
48. Helicobacter pylori metalloprotease gene, complete cds (621 bp linear DNA, AF380136.1 GI: 21310088).
49. Staphylococcus chromogenes metalloprotease (scp) gene, complete cds (1,506 bp linear DNA,
AF218055.1 GI: 6942069)
50. Vibrio cholerae collagenase (vcc) gene, complete cds (2,934 bp linear DNA, AF080248.1 GI: 3925384).
51. WO 2019187691-A/1: POLYPEPTIDE HAVING COLLAGENASE ACTIVITY AND PRODUCTION
(934 bp linear DNA, MB471429.1 GI: 1803042352).
52. Pseudomonas baetica aprA gene for aprA metalloprotease, isolate a390 (1,403 bp linear DNA,
LR736275.1 GI: 1795714649).
53. XXX (11,022 bp linear DNA, LR589271.1 GI: 1795669102).
54. XXX (10,918 bp linear DNA LR589191.1 GI: 1795667112).
55. Ganoderma boninense I6X8R2 gene for Probable zinc metalloprotease Zmp1 (1,944 bp linear DNA,
LR727177.1 GI: 1768658458).
56. Ganoderma boninense Q49GQ5 gene for Secreted alkaline metalloprotease (398 bp linear DNA,
LR725042.1 GI: 1768655205).
57. Ganoderma boninense C5P3X6 gene for Extracellular metalloprotease 1 (EC 3.4.24.-) (1,131 bp linear
DNA, LR724190.1 GI: 1768652831).
58. Lactobacillus helveticus strain GY-3 aminopeptidase I zinc metalloprotease (LAC_GM000834) gene,
complete cds (1,113 bp linear DNA, MK675051.1 GI: 1764573391).
59. Streptococcus gordonii strain SK186 CamG (camG), amino glycoside 6-adenylyltransferase, and CPBP
family intramembrane metalloprotease (ydiL) genes, complete cds (2,189 bp linear DNA, MH723708.1
GI: 1735625106).
60. Streptococcus gordonii strain SK86 CamG (camG), amino glycoside 6-adenylyltransferase, and CPBP
family intramembrane metalloprotease (ydiL) genes, complete cds (2,160 bp linear DNA, MH723707.1
GI: 1735625102).
61. Serratia plymuthica strain UBCF_13 metalloprotease gene, complete cds (1,059 bp linear DNA,
MK524938.1 GI: 1733436778).
62. Serratia plymuthica strain UBCF_01 metalloprotease gene, complete cds (1,059 bp linear DNA,
MK524937.1 GI: 1733436776).
63. Serratia plymuthica strain UBCR_12 metalloprotease gene, complete cds (1,059 bp linear DNA,
MK524936.1 GI: 1733436774).
64. Uncultured bacterium clone O5_aCas9_16 genomic sequence (1,243 bp linear DNA, MK637573.1
GI: 1728392661).
65. Homo sapiens TIMP metallopeptidase inhibitor 1 (TIMP1), RefSeqGene on chromosome X (11,501 bp
linear DNA, NG_012533.1 GI: 254911137).
66. Uncultured organism clone KBTEX_165 genomic sequence (6,630 bp linear DNA, MN079240.1
GI: 1720646339).
67. Uncultured organism clone KBTEX_155 genomic sequence (7,393 bp linear DNA, MN079230.1
GI: 1720646268)
68. Uncultured organism clone KBTEX_121 genomic sequence (10,305 bp linear DNA, MN079196.1
GI: 1720645953).
69. Bacillus cereus strain MH19 peptidase U32 gene, complete cds (930 bp linear DNA, MK389496.1
GI: 1718349207).
70. Bacillus cereus strain MH19 peptidase U32 gene, complete cds (1,281 bp linear DNA, MK389495.1
GI: 1718349205).
71. Bacillus cereus strain MH19 peptidase S8 gene, complete cds (1,842 bp linear DNA, MK389494.1
GI: 1718349203).
72. Bacillus cereus strain MH19 peptidase M9 gene, complete cds (2,898 bp linear DNA, MK389493.1
GI: 1718349201).
73. Bacillus cereus strain MH19 peptidase M9 gene, complete cds (2,916 bp linear DNA, MK389492.1
GI: 1718349199).
74. Proteus mirabilis strain 5PMK/SRLAAH/2018 metalloprotease gene, complete cds (1,754 bp linear
DNA, MK284532.1 GI: 1702223766).
75. Streptomyces mozunensis strain MK-23 phosphoramidon gene cluster, complete sequence; and TalB
(talB) gene, complete cds (7,021 bp linear DNA, MK644118.1 GI: 1654333832).
76. Anopheles culicifacies A aminopeptidase N 1 gene, complete cds (3,828 bp linear DNA, MK033514.1
GI: 1635477658).
77. UNVERIFIED: Serratia marcescens strain S6 metalloprotease-like gene, complete sequence (1,371 bp
linear DNA, MK018002.1 GI: 1632161445).
78. Vibrio coralliilyticus strain OCN008 VtpA (vtpA) gene, complete cds (1,824 bp linear DNA,
MH794510.1 GI: 1611977484).
79. Pleurotus salmoneostramineus NBRC 31859 gene for metalloprotease, complete cds (2,279 bp linear
DNA, LC467535.1 GI: 1594459819)
80. 960 Flavobacterium columnare genomic DNA SSH library Flavobacterium columnare genomic 5′ similar
to Zinc metalloprotease, genomic survey sequence (283 bp linear DNA, FI188228.1 GI: 297124726).
81. M3F85 Listonella anguillarum strain M3 fosmid library Vibrio anguillarum M3 genomic clone
M3FR8D04 similar to metalloprotease, genomic survey sequence (673 bp linear DNA, FI185189.1
GI: 225579011).
82. Salinivibrio sp. YH4 metalloprotease YHM gene, complete cds (1,836 bp linear DNA, MK105901.1
GI: 1558328208).
83. Pseudoalteromonas sp. strain SJ2 metalloprotease J2 gene, complete cds (2,184 bp linear DNA,
MK105899.1 GI: 1558327534).
84. Xenorhabdus bovienii strain xbm1 metalloprotease (mp) gene, complete cds (1,428 bp linear DNA,
FJ624425.1 GI: 255969435).
85. Sequence 1 from U.S. Pat. No. 1,010,6761 (2,950 bp linear DNA, MM106142.1 GI: 1531377966).
86. Escherichia coli strain 10-188 plasmid p10-188 clone c10 genomic sequence (4,956 bp linear DNA,
MH847540.1 GI: 1511117605).
87. Escherichia coli strain 15-313 plasmid p15-313 clone c4 genomic sequence (6,541 bp linear DNA,
MH846992.1 GI: 1511077344).
88. Escherichia coli strain 10-396 plasmid p10-396 clone c3 genomic sequence (6,600 bp linear DNA,
MH846964.1 GI: 1511077112).
89. Sequence 2 from U.S. Pat. No. 1,004,7353 (2,304 bp linear DNA, MI946437.1 GI: 1489267642).
90. Serratia marcescens strain K904 serralysin-family cytotoxic metalloprotease (slpD) gene, complete cds
(1,617 bp linear DNA, MG020515.1 GI: 1464276236).
91. Serratia marcescens strain K904 serralysin-family cytotoxic metalloprotease (slpC) gene, complete cds
(1,383 bp linear DNA, MG020514.1 GI: 1464276234).
92. Serratia marcescens strain K904 serralysin-family cytotoxic metalloprotease (slpB) gene, complete cds
(1,419 bp linear DNA, MG020513.1 GI: 1464276232).
93. Serratia marcescens strain K904 serralysin-family cytotoxic metalloprotease (prtS) gene, complete cds
(1,515 bp linear DNA, MG020512.1 GI: 1464276230).
94. B.jararaca mRNA for jararhagin (2,118 bp linear DNA, X68251.1 GI: 62467)
95. Clostridium botulinum strain Templin plasmid botulinum neurotoxin type B (bont/B) gene, complete cds
(3,876 bp linear DNA, MG545727.1 GI: 1388193544).
96. Streptomyces platensis strain CB00739 PtmS1 (ptmS1) gene, complete cds (426 bp linear DNA,
MG265942.1 GI: 1371543683).
97. Vibrio furnissii strain KCCM 41679 metalloprotease (vFMP) gene, complete cds (1,827 bp linear DNA,
MG954380.1 GI: 1357237960).
98. Vibrio cholerae strain R-18963 collagenase gene, complete cds, (2,430 bp linear DNA, MF100095.1
GI: 1354638853).
99. Vibrio cholerae strain R-18904 collagenase gene, complete cds (2,430 bp linear DNA, MF100094.1
GI: 1354638851).
100. Vibrio cholerae strain R-18588 collagenase gene, complete cds (2,430 bp linear DNA, MF100093.1
GI: 1354638849).
101. Vibrio cholerae strain R-18252 collagenase gene, complete cds (2,430 bp linear DNA, MF100092.1
GI: 1354638847).
102. Vibrio cholerae strain R-13169 collagenase gene, complete cds (2,430 bp linear DNA, MF100091.1
GI: 1354638845).
103. Vibrio cholerae strain R-81 collagenase gene, complete cds (2,430 bp linear DNA, MF100090.1
GI: 1354638843).
104. JP 2017121234-A/10: Use and Production of Storage-Stable Neutral Metalloprotease (1,566 bp linear
DNA, LX296980.1 GI: 1271810730).
105. JP 2017121234-A/2: Use and Production of Storage-Stable Neutral Metalloprotease (1,578 bp linear
DNA, LX296972.1 GI: 1271810722).
106. JP 2017121234-A/1: Use and Production of Storage-Stable Neutral Metalloprotease (713 bp linear
DNA, LX296971.1 GI: 1271810721).
107. KR 1020160131037-A/2: GRIMONTIA-HOLLISAE-DERIVED RECOMBINANT COLLAGENASE
AND ENZYME AGENT FOR CELL SEPARATION, 2,304 bp linear DNA, LY409089.1 GI: 1257727869.
108. Flavobacterium columnare clone T9SS-45 M4 family metalloprotease precursor, gene, complete cds,
2,988 bp linear DNA, MF535439.1 GI: 1242932190.
109. Flavobacterium columnare clone T9SS-43 zinc metalloprotease precursor subfamily M43a peptidase
gene, complete cds, 1,317 bp linear DNA, MF535437.1 GI: 1242932091.
110. Flavobacterium columnare clone T9SS-41 metalloprotease M12b family precursor, gene, complete cds,
3,231 bp linear DNA, MF535435.1 GI: 1242931975.
111. Flavobacterium columnare clone T9SS-36 putative thermolysin M4 family metalloprotease precursor,
gene, complete cds, 2,709 bp linear DNA, MF535430.1 GI: 1242931718.
112. Flavobacterium columnare clone T9SS-30 putative collagenase precursor subfamily M43a peptidase
gene, complete cds, 3,045 bp linear DNA, MF535424.1 GI: 1242931384.
113. Flavobacterium columnare clone T9SS-29 zinc-dependent metalloprotease gene, complete cds, 1,269 bp
linear DNA, MF535423.1 GI: 1242931334.
114. Flavobacterium columnare clone T9SS-28 zinc-dependent metalloprotease M43 family pappalysin-like
subfamily protein gene, complete cds, 2,832 bp linear DNA, MF535422.1 GI: 1242931281.
115. Flavobacterium columnare clone T9SS-19 zinc-dependent metalloprotease M36 fungalysin family
precursor, gene, complete cds, 2,733 bp linear DNA, MF535413.1 GI: 1242930936.
116. Flavobacterium columnare clone T9SS-14 zinc-dependent metalloprotease precursor M4/M36 family
protein gene, complete cds, 2,049 bp linear DNA, MF535408.1 GI: 1242930622.
117. Pseudoalteromonas piscicida mprIII gene for metalloprotease III, complete cds, 4,610 bp linear DNA,
AB084466.1 GI: 24460065.
118. Uncultured bacterium clone fosmid F383-385 genomic sequence, 2,500 bp linear DNA, KX576137.1
GI: 1197346184.
119. Uncultured bacterium clone pCAW6 genomic sequence, 2,125 bp linear DNA. KY939594.1
GI: 1194412178.
120. KR 1020160040344-A/1: Triclosan exposure to changes responsive gene in Strongylocentrotus nudus
and the method for diagnosing marine ecosystem using the same, 2,297 bp linear DNA, LG146000.1
GI: 1139296047.
121. Homo sapiens matrilysin gene, complete cds, 1,721 bp linear DNA, AH006461.2 GI: 1134617879.
122. Uncultured bacterium clone Control_TwinB_Time2_CL_58 genomic sequence, 2,146 bp linear DNA,
KX128572.1 GI: 1120617331.
123. Uncultured bacterium clone AmoxDisc_Mom_TR-SX 22 genomic sequence, 5,500 bp linear DNA,
KX125928.1 GI: 1120608655
124. Sequence 12 from U.S. Pat. No. 9,334,467, 1,566 bp linear DNA, HL810280.1 GI: 1115485643
125. Sequence 2 from U.S. Pat. No. 9,334,467, 1,578 bp linear DNA, HL810272.1 GI: 1115485635
126. Sequence 1 from U.S. Pat. No. 9,334,467, 713 bp linear DNA HL810271.1 GI: 1115485634
127. Pseudoalteromonas sp. strain CSN423-M metalloprotease gene, complete cds, 2,566 bp linear DNA
KX458247.1 GI: 1092945061
128. Pseudoalteromonas sp. strain CSN423 metalloprotease gene, complete cds 2,566 bp linear DNA
KX458246.1 GI: 1092945059
129. Uncultured bacterium clone pR-2-2 genomic sequence 3,920 bp linear DNA KT860436.1
GI: 1063798479
130. Uncultured bacterium clone pQ2N genomic sequence 3,838 bp linear DNA KT860434.1
GI: 1063798472
131. Sequence 6 from U.S. Pat. No. 9,211,316 3,066 bp linear DNA HL445047.1 GI: 1060773987
132. Sequence 4 from U.S. Pat. No. 9,211,316 330 bp linear DNA HL445046.1 GI: 1060773986
133. Sequence 3 from U.S. Pat. No. 9,211,316 3,357 bp linear DNA HL445045.1 GI: 1060773985
134. Homo sapiens Human type IV collagenase (CLG4B) gene 4,371 bp linear DNA AH001496.2
GI: 1059793967
135. Homo sapiens type IV collagenase (CLG4A) gene, complete cds 4,925 bp linear DNA AH002654.2
GI: 1049010829
136. Mutant Bacillus anthracis strain 34F2 plasmid PX O1 mutant lethal factor (mlef) gene, complete cds
2,430 bp linear DNA JQ798177.1 GI: 390132840
137. Salmonella enteritidis pathogenicity islet and unknown genes 8,095 bp linear DNA AF128839.1
GI: 7229294
138. gelatinase = matrix metalloprotease/enterotoxin [Bacteroides fragilis, VPI 13784, Genomic, 538 nt] 538
bp linear DNA S75941.1 GI: 913134
139. Escherichia coli strain EB260 YghJ (yghJ) gene, complete cds 4,723 bp linear DNA KX245009.1
GI: 1043526309
140. Oryctolagus cuniculus strain New England White collagenase-1 precursor (MMP-1) gene, complete cds
8,664 bp linear DNA AH005676.2 GI: 1036032494
141. Homo sapiens chromosome 3 tissue inhibitor of metalloproteinase 4 (TIMP4) gene, complete cds 2,379
bp linear DNA AH006411.2 GI: 1036030230
142. JP 2016019518-A/10: Use and Production of Storage-Stable Neutral Metalloprotease 1,566 bp linear
DNA HZ489807.1 GI: 1032158727
143. JP 2016019518-A/2: Use and Production of Storage-Stable Neutral Metalloprotease 1,578 bp linear
DNA HZ489799.1 GI: 1032158719
144. JP 2016019518-A/1: Use and Production of Storage-Stable Neutral Metalloprotease 713 bp linear DNA
HZ489798.1 GI: 1032158718
145. Eutrema salsugineum gene for ATP-dependent zinc metalloprotease ThFtsH8, complete cds 2,228 bp
linear DNA AB598414.1 GI: 311893428
146. Aeromonas piscicola strain AH-3 collagenase (colAh) gene, complete cds 2,748 bp linear DNA
JQ639076.1 GI: 399762992
147. Vibrio alginolyticus chemovar iophagus varA gene for response regulator, complete cds, strain: I.029
645 bp linear DNA LC146713.1 GI: 1019366631
148. Vibrio alginolyticus chemovar iophagus varS gene for sensor histidine kinase, complete cds, strain:
I.029 2,799 bp linear DNA LC146712.1 GI: 1019366629
149. Bacillus cereus strain Col15 collagenase gene, complete cds 2,898 bp linear DNA KT593866.1
GI: 999991863
150. Vibrio mimicus vmp gene, promoter region, strain: ES-37 507 bp linear DNA LC099949.1
GI: 957657114
151. Serratia liquefaciens strain LMG26066 Ser2 (ser2) gene, complete cds 2,368 bp linear DNA
KR935799.1 GI: 946731838
152. Serratia liquefaciens strain LMG26065 Ser2 (ser2) gene, complete cds 2,368 bp linear DNA
KR935798.1 GI: 946731836
153. Serratia liquefaciens strain ATCC51814 Ser2 (ser2) gene, complete cds 2,308 bp linear DNA
KR935797.1 GI: 946731834
154. Serratia liquefaciens strain ATCC25642 Ser2 (ser2) gene, complete cds 2,367 bp linear DNA
KR935796.1 GI: 946731832
155. Serratia liquefaciens strain DSM30066 Ser2 (ser2) gene, complete cds 2,396 bp linear DNA
KR935795.1 GI: 946731830
156. Serratia liquefaciens strain ATCC 27592 Ser2 (ser2) gene, complete cds 2,366 bp linear DNA
KR935794.1 GI: 946731828
157. Serratia liquefaciens strain L153 Ser2 (ser2) gene, complete cds 2,370 bp linear DNA KR935793.1
GI: 946731826
158. Serratia liquefaciens strain L146 Ser2 (ser2) gene, complete cds 2,377 bp linear DNA KR935792.1
GI: 946731824
159. Serratia liquefaciens strain L140 Ser2 (ser2) gene, complete cds 2,368 bp linear DNA KR935791.1
GI: 946731822
160. Serratia liquefaciens strain L137 Ser2 (ser2) gene, complete cds 2,388 bp linear DNA KR935790.1
GI: 946731820
161. Serratia liquefaciens strain L136 Ser2 (ser2) gene, complete cds 2,368 bp linear DNA KR935789.1
GI: 946731818
162. Serratia liquefaciens strain L135 Ser2 (ser2) gene, complete cds 2,366 bp linear DNA KR935788.1
GI: 946731816
163. Serratia liquefaciens strain L132 Ser2 (ser2) gene, complete cds 2,376 bp linear DNA KR935787.1
GI: 946731814
164. Serratia liquefaciens strain L130 Ser2 (ser2) gene, complete cds 2,366 bp linear DNA KR935786.1
GI: 946731812
165. Serratia liquefaciens strain L128 Ser2 (ser2) gene, complete cds 2,376 bp linear DNA KR935785.1
GI: 946731810
166. Serratia liquefaciens strain L113 Ser2 (ser2) gene, complete cds 2,367 bp linear DNA KR935784.1
GI: 946731808
167. Serratia liquefaciens strain L104 Ser2 (ser2) gene, complete cds 2,367 bp linear DNA KR935783.1
GI: 946731806
168. Serratia liquefaciens strain L98 Ser2 (ser2) gene, complete cds 2,346 bp linear DNA KR935782.1
GI: 946731804
169. Serratia liquefaciens strain L95 Ser2 (ser2) gene, complete cds 2,368 bp linear DNA KR935781.1
GI: 946731802
170. Serratia liquefaciens strain L79 Ser2 (ser2) gene, complete cds 2,380 bp linear DNA KR935780.1
GI: 946731800
171. Serratia liquefaciens strain L64 Ser2 (ser2) gene, complete cds 2,377 bp linear DNA KR935779.1
GI: 946731798
172. Serratia liquefaciens strain L61 Ser2 gene, complete cds 2,379 bp linear DNA KR935778.1
GI: 946731796
173. Serratia liquefaciens strain L53 Ser2 (ser2) gene, complete cds 2,357 bp linear DNA KR935777.1
GI: 946731794
174. Paenibacillus lautus ATP-independent zinc metalloprotease M50Ppl gene, complete cds 693 bp linear
DNA KM099426.1 GI: 782977319
175. Ralstonia pickettii metalloprotease RpA gene, complete cds 1,449 bp linear DNA KP225110.1
GI: 805486991
176. Flavobacterium psychrophilum DNA, collagenase pseudogene, isolate: WB-1 2,774 bp linear DNA
AB921328.1 GI: 765716002
177. Flavobacterium psychrophilum gene for collagenase, complete cds, isolate: WA-2 2,886 bp linear DNA
AB921327.1 GI: 765715998
178. Flavobacterium psychrophilum gene for collagenase, complete cds, isolate: WA-1 2,883 bp linear DNA
AB921326.1 GI: 765715995
179. Flavobacterium psychrophilum DNA, fpp2 pseudogene, transposon, isolate: WB-1 3,848 bp linear DNA
AB921325.1 GI: 765715992
180. Flavobacterium psychrophilum DNA, fpp2 pseudogene, isolate: WA-2 2,832 bp linear DNA
AB921324.1 GI: 765715987
181. Flavobacterium psychrophilum DNA, fpp2 pseudogene, transposon, isolate: WA-1 3,834 bp linear DNA
AB921323.1 GI: 765715983
182. Flavobacterium psychrophilum fppl gene for metalloprotease, complete cds, isolate: WB-1 3,417 bp
linear DNA AB921322.1 GI: 765715977
183. Flavobacterium psychrophilum fpp1 gene for metalloprotease, complete cds, isolate: WA-2 3,420 bp
linear DNA AB921321.1 GI: 765715972
184. Flavobacterium psychrophilum DNA, fpp1 pseudogene, isolate: WA-1 3,416 bp linear DNA
AB921320.1 GI: 765715969
185. Vibrio aestuarianus clone 12830515 secreted zinc metalloprotease Vam (vam) gene, complete cds 1,836
bp linear DNA KM588637.1 GI: 724471583
186. KR 1020140027423-A/1: USE AND PRODUCTION OF STORAGE-STABLE NEUTRAL
METALLOPROTEASE 713 bp linear DNA DI401411.1 GI: 724438585
187. KR 1020140027423-A/12: USE AND PRODUCTION OF STORAGE-STABLE NEUTRAL
METALLOPROTEASE 1,566 bp linear DNA DI401420.1 GI: 724438482
188. KR 1020140027423-A/2: USE AND PRODUCTION OF STORAGE-STABLE NEUTRAL
METALLOPROTEASE 1,578 bp linear DNA DI401412.1 GI: 724438480
189. Verrucosispora sp. MS100047 clone cluster9_9330_16959 hypothetical protein (VASRM7_657),
hypothetical protein (VASRM7_654), ArsR family transcriptional regulator (VASRM7_655), hypothetical
protein (VASRM7_656), and putative metalloprotease (VASRM7_658) genes, complete cds 7,628 bp linear
DNA KF826707.1 GI: 695117661
190. Trichophyton mentagrophytes MEP4 (MEP4) gene, complete cds 2,185 bp linear DNA KF751729.1
GI: 689297218
191. Trichophyton mentagrophytes MEP3 (MEP3) gene, complete cds 2,241 bp linear DNA KF751728.1
GI: 689297212
192. WO 2001034785-A/30: Novel metalloprotease having an activity of aggrecanase 3,455 bp linear DNA
BD095180.1 GI: 22640768
193. WO 2001034785-A/29: Novel metalloprotease having an activity of aggrecanase 3,462 bp linear DNA
BD095179.1 GI: 22640767
194. WO 2001034785-A/28: Novel metalloprotease having an activity of aggrecanase 3,467 bp linear DNA
BD095178.1 GI: 22640766
195. WO 2001034785-A/27: Novel metalloprotease having an activity of aggrecanase 3,470 bp linear DNA
BD095177.1 GI: 22640765
196. WO 2001034785-A/26: Novel metalloprotease having an activity of aggrecanase 3,469 bp linear DNA
BD095176.1 GI: 22640764
197. WO 2001034785-A/25: Novel metalloprotease having an activity of aggrecanase 3,464 bp linear DNA
BD095175.1 GI: 22640763
198. WO 2001034785-A/24: Novel metalloprotease having an activity of aggrecanase 3,467 bp linear DNA
BD095174.1 GI: 22640762
199. WO 2001034785-A/23: Novel metalloprotease having an activity of aggrecanase 3,473 bp linear DNA
BD095173.1 GI: 22640761
200. WO 2001034785-A/1: Novel metalloprotease having an activity of aggrecanase 2,853 bp linear DNA
BD095151.1 GI: 22640739
201. JP 2014064562-A/10: Use and Production of Storage-Stable Neutral Metalloprotease 1,566 bp linear
DNA HW509728.1 GI: 669239204
202. JP 2014064562-A/2: Use and Production of Storage-Stable Neutral Metalloprotease 1,578 bp linear
DNA HW509720.1 GI: 669239196
203. JP 2014064562-A/1: Use and Production of Storage-Stable Neutral Metalloprotease 713 bp linear DNA
HW509719.1 GI: 669239195
204. Morganella morganii strain ZM putative metalloprotease gene, complete cds 1,344 bp linear DNA
KJ649438.1 GI: 636571990
205. Aeromonas hydrophila strain 450 metalloprotease gene, complete cds 1,773 bp linear DNA JQ613372.1
GI: 386785840
206. Aeromonas hydrophila strain 438 metalloprotease gene, complete cds 1,773 bp linear DNA JQ613371.1
GI: 386785838
207. Aeromonas hydrophila strain 434 metalloprotease gene, complete cds 1,773 bp linear DNA JQ613370.1
GI: 386785836
208. Aeromonas hydrophila strain 431 metalloprotease gene, complete cds 1,773 bp linear DNA JQ613369.1
GI: 386785834
209. Aeromonas hydrophila strain 421 metalloprotease gene, complete cds 1,773 bp linear DNA JQ613368.1
GI: 386785832
210. Aeromonas hydrophila strain 433 metalloprotease gene, complete cds 1,770 bp linear DNA JQ613367.1
GI: 386785830
211. Aeromonas hydrophila strain 428 metalloprotease gene, complete cds 1,770 bp linear DNA JQ613366.1
GI: 386785828
212. Aeromonas hydrophila strain 405 metalloprotease gene, complete cds 1,770 bp linear DNA JQ613365.1
GI: 386785826
213. Aeromonas hydrophila strain 406 metalloprotease gene, complete cds 1,770 bp linear DNA JQ613364.1
GI: 386785824
214. Aeromonas hydrophila strain 412 metalloprotease gene, complete cds 1,770 bp linear DNA JQ613363.1
GI: 386785822
215. Aeromonas hydrophila strain 451 metalloprotease gene, complete cds 1,770 bp linear DNA JQ613362.1
GI: 386785820
216. Aeromonas hydrophila strain 453 metalloprotease gene, complete cds 1,770 bp linear DNA JQ613361.1
GI: 386785818
217. Pseudomonas aeruginosa strain CTM50182 elastase precursor (lasB) gene, complete cds 1,592 bp linear
DNA JX970630.1 GI: 480311130
218. Anopheles culicifacies C carboxypeptidase A gene, complete cds 2,994 bp linear DNA KJ002442.1
GI: 594542594
219. Sequence 13 from U.S. Pat. No. 8,617,543 2,979 bp linear DNA HJ695516.1 GI: 576942026
220. Sequence 12 from U.S. Pat. No. 8,617,543 2,946 bp linear DNA HJ695515.1 GI: 576942025
221. Vibrio tubiashii strain 07/118-T2 metalloprotease gene, complete cds 1,821 bp linear DNA KF270512.1
GI: 574609374
222. Uncultured bacterium Contig1536 genomic sequence 3,117 bp linear DNA KC247046.1 GI: 571053568
223. Uncultured bacterium Contig1285 genomic sequence 3,289 bp linear DNA KC247019.1 GI: 571053433
224. Vibrio coralliilyticus strain CI zinc metalloprotease (vcpA) gene, complete cds 1,824 bp linear DNA
JQ345046.1 GI: 387861973
225. Vibrio coralliilyticus P1 zinc metalloprotease (vcpA) gene, complete cds 1,824 bp linear DNA
JQ345045.1 GI: 387861971
226. Vibrio coralliilyticus strain C2 zinc metalloprotease (vcpA) gene, complete cds 1,824 bp linear DNA
JQ345044.1 GI: 387861969
227. Vibrio coralliilyticus strain P2 zinc metalloprotease (vcpA) gene, complete cds 1,824 bp linear DNA
JQ345043.1 GI: 387861967
228. Vibrio coralliilyticus strain P4 zinc metalloprotease (vcpA) gene, complete cds 1,824 bp linear DNA
JQ345042.1 GI: 387861965
229. Vibrio coralliilyticus strain P5 zinc metalloprotease (vcpA) gene, complete cds 1,824 bp linear DNA
JQ345041.1 GI: 387861963
230. Vibrio coralliilyticus strain P6 zinc metalloprotease (vcpA) gene, complete cds 1,824 bp linear DNA
JQ345040.1 GI: 387861961
231. Vibrio coralliilyticus strain Tav24 zinc metalloprotease (vcpA) gene, complete cds 1,824 bp linear DNA
JQ345039.1 GI: 387861959
232. Vibrio coralliilyticus strain BH1 zinc metalloprotease (vcpA) gene, complete cds 1,824 bp linear DNA
JQ345038.1 GI: 387861957
233. Vibrio coralliilyticus strain BH2 zinc metalloprotease (vcpA) gene, complete cds 1,824 bp linear DNA
JQ345037.1 GI: 387861955
234. Vibrio coralliilyticus strain BH3 zinc metalloprotease (vcpA) gene, complete cds 1,824 bp linear DNA
JQ345036.1 GI: 387861953
235. Vibrio coralliilyticus strain BH4 zinc metalloprotease (vcpA) gene, complete cds 1,824 bp linear DNA
JQ345035.1 GI: 387861951
236. Vibrio coralliilyticus strain BH5 zinc metalloprotease (vcpA) gene, complete cds 1,824 bp linear DNA
JQ345034.1 GI: 387861949
237. Vibrio coralliilyticus strain BH6 zinc metalloprotease (vcpA) gene, complete cds 1,824 bp linear DNA
JQ345033.1 GI: 387861947
238. JP 2013527756-A/29: Genetic Signatures and Gene Chips Associated with administration of Electrically
Conducted Radio Frequency Current to Skin and Methods and Treatments Relating Thereto 2,387 bp linear
DNA HW313792.1 GI: 559095693
239. JP 2013527756-A/28: Genetic Signatures and Gene Chips Associated with administration of Electrically
Conducted Radio Frequency Current to Skin and Methods and Treatments Relating Thereto 1,973 bp linear
DNA HW313791.1 GI: 559095692
240. Serratia marcescens ser gene for serralysin metalloprotease, complete cds, strain: 2170 1,482 bp linear
DNA AB873002.1 GI: 558610979
241. Serratia liquefaciens ser2 gene for serralysin-like metalloprotease 2, complete cds, strain: Kuo1-1 2,009
bp linear DNA AB638721.1 GI: 336088535
242. Serratia liquefaciens ser1, inh genes for serralysin-like metalloprotease 1, protease inhibitor, complete
cds, strain: Kuo1-1 3,278 bp linear DNA AB638720.1 GI: 336088532
243. Vibrio vulnificus gene for alkyl sulfatase, complete cds, strain: CECT5198 1,968 bp linear DNA
AB856281.1 GI: 548923652
244. Vibrio vulnificus gene for hypothetical protein, complete cds, strain: CECT5198 903 bp linear DNA
AB856280.1 GI: 548923650
245. Serratia sp. F1390 metalloprotease gene, complete cds 1,515 bp linear DNA KF372856.1 GI: 541129133
246. UNVERIFIED: Acacia auriculiformis isolate KUABAA54 hydrolase-like gene, complete sequence 878
bp linear DNA KC193587.1 GI: 507899240
247. Sequence 5 from U.S. Pat. No. 8,389,284 2,262 bp linear DNA GZ841069.1 GI: 507877687
248. Sequence 3 from U.S. Pat. No. 8,389,284 2,340 bp linear DNA GZ841068.1 GI: 507877686
249. Sequence 1 from U.S. Pat. No. 8,389,284 2,076 bp linear DNA GZ841067.1 GI: 507877685
250. Streptococcus pneumoniae strain SP168 zinc metalloprotease C (zmpC) gene, complete cds 4,848 bp
linear DNA JQ396430.1 GI: 379334187
251. Streptomyces mobaraensis mp2 gene for metalloprotease 2, strain DSM 40847 1,611 bp linear DNA
HF968454.1 GI: 487395534
252. Streptomyces mobaraensis mp1 gene for metalloprotease 1, strain DSM 40847 1,557 bp linear DNA
HF968453.1 GI: 487395531
253. Streptomyces mobaraensis tamep gene for transglutaminase-activating metalloprotease, strain DSM
40847 2,283 bp linear DNA HF968452.1 GI: 487395529
254. Streptomyces mobaraensis sti gene for subtilisin and transglutaminase-activating metalloprotease
inhibitor, strain DSM 40847 447 bp linear DNA HF968451.1 GI: 487395527
255. Trypanosoma rangeli strain P07 major surface protease gene, complete cds 1,767 bp linear DNA
JQ579649.1 GI: 409107867
256. Sequence 27 from Patent WO2009125295 5,379 bp linear DNA JA863243.1 GI: 448979704
257. Sequence 26 from Patent WO2009125295 5,379 bp linear DNA JA863242.1 GI: 448979703
258. Streptococcus australis strain FRStet12 ABC transporter subunit A (steA), ABC transporter subunit B
(steB), putative metalloprotease, putative diacylglycerol kinase, and putative GTP-binding protein Era genes,
complete cds 6,318 bp linear DNA HQ652506.1 GI: 356472762
259. Pseudoalteromonas sp. CF6-2 elastinolytic metalloprotease gene, complete cds 1,212 bp linear DNA
HQ005379.1 GI: 315570438
260. Pseudomonas aeruginosa strain ATCC 27853 alkaline metalloprotease (aprA) gene, complete cds 1,511
bp linear DNA JX853450.1 GI: 424719213
261. Pseudomonas aeruginosa strain PAO2/18 alkaline metalloprotease (aprA) gene, complete cds 1,507 bp
linear DNA JX853449.1 GI: 424719211
262. Pseudomonas aeruginosa strain PAC124/9 alkaline metalloprotease (aprA) gene, complete cds 1,512 bp
linear DNA JX853448.1 GI: 424719209
263. JP 2011516081-A/28: THROMBIN ACTIVATOR COMPOSITIONS AND METHODS OF MAKING
AND USING THE SAME 1,455 bp linear DNA HV932664.1 GI: 415676474
264. JP 2011516081-A/27: THROMBIN ACTIVATOR COMPOSITIONS AND METHODS OF MAKING
AND USING THE SAME 1,302 bp linear DNA HV932663.1 GI: 415676470
265. JP 2011516081-A/26: THROMBIN ACTIVATOR COMPOSITIONS AND METHODS OF MAKING
AND USING THE SAME 1,317 bp linear DNA HV932662.1 GI: 415676467
266. JP 2011516081-A/25: THROMBIN ACTIVATOR COMPOSITIONS AND METHODS OF MAKING
AND USING THE SAME 2,073 bp linear DNA HV932661.1 GI: 415676463
267. JP 2011516081-A/24: THROMBIN ACTIVATOR COMPOSITIONS AND METHODS OF MAKING
AND USING THE SAME 1,869 bp linear DNA HV932660.1 GI: 415676460
268. JP 2011516081-A/23: THROMBIN ACTIVATOR COMPOSITIONS AND METHODS OF MAKING
AND USING THE SAME 711 bp linear DNA HV932659.1 GI: 415676455
269. JP 2011516081-A/22: THROMBIN ACTIVATOR COMPOSITIONS AND METHODS OF MAKING
AND USING THE SAME 1,353 bp linear DNA HV932658.1 GI: 415676452
270. JP 2011516081-A/21: THROMBIN ACTIVATOR COMPOSITIONS AND METHODS OF MAKING
AND USING THE SAME 1,869 bp linear DNA HV932657.1 GI: 415676449
271. JP 2011516081-A/20: THROMBIN ACTIVATOR COMPOSITIONS AND METHODS OF MAKING
AND USING THE SAME 2,073 bp linear DNA HV932656.1 GI: 415676446
272. JP 2011516081-A/19: THROMBIN ACTIVATOR COMPOSITIONS AND METHODS OF MAKING
AND USING THE SAME 1,902 bp linear DNA HV932655.1 GI: 415676443
273. JP 2011516081-A/18: THROMBIN ACTIVATOR COMPOSITIONS AND METHODS OF MAKING
AND USING THE SAME 2,205 bp linear DNA HV932654.1 GI: 415676435
274. JP 2011516081-A/17: THROMBIN ACTIVATOR COMPOSITIONS AND METHODS OF MAKING
AND USING THE SAME 1,950 bp linear DNA HV932653.1 GI: 415676432
275. JP 2011516081-A/16: THROMBIN ACTIVATOR COMPOSITIONS AND METHODS OF MAKING
AND USING THE SAME 1,558 bp linear DNA HV932652.1 GI: 415676429
276. JP 2011516081-A/15: THROMBIN ACTIVATOR COMPOSITIONS AND METHODS OF MAKING
AND USING THE SAME 901 bp linear DNA HV932651.1 GI: 415676426
277. JP 2011516081-A/14: THROMBIN ACTIVATOR COMPOSITIONS AND METHODS OF MAKING
AND USING THE SAME 1,233 bp linear DNA HV932650.1 GI: 415676423
278. JP 2011516081-A/13: THROMBIN ACTIVATOR COMPOSITIONS AND METHODS OF MAKING
AND USING THE SAME 1,498 bp linear DNA HV932649.1 GI: 415676420
279. JP 2011516081-A/12: THROMBIN ACTIVATOR COMPOSITIONS AND METHODS OF MAKING
AND USING THE SAME 1,869 bp linear DNA HV932648.1 GI: 415676417
280. JP 2011516081-A/11: THROMBIN ACTIVATOR COMPOSITIONS AND METHODS OF MAKING
AND USING THE SAME 1,887 bp linear DNA HV932647.1 GI: 415676414
281. JP 2011516081-A/10: THROMBIN ACTIVATOR COMPOSITIONS AND METHODS OF MAKING
AND USING THE SAME 1,923 bp linear DNA HV932646.1 GI: 415676410
282. JP 2011516081-A/9: THROMBIN ACTIVATOR COMPOSITIONS AND METHODS OF MAKING
AND USING THE SAME 1,977 bp linear DNA HV932645.1 GI: 415676407
283. JP 2011516081-A/8: THROMBIN ACTIVATOR COMPOSITIONS AND METHODS OF MAKING
AND USING THE SAME 750 bp linear DNA HV932644.1 GI: 415676404
284. JP 2011516081-A/7: THROMBIN ACTIVATOR COMPOSITIONS AND METHODS OF MAKING
AND USING THE SAME 765 bp linear DNA HV932643.1 GI: 415676401
285. JP 2011516081-A/6: THROMBIN ACTIVATOR COMPOSITIONS AND METHODS OF MAKING
AND USING THE SAME 1,422 bp linear DNA HV932642.1 GI: 415676398
286. JP 2011516081-A/5: THROMBIN ACTIVATOR COMPOSITIONS AND METHODS OF MAKING
AND USING THE SAME 1,407 bp linear DNA HV932641.1 GI: 415676395
287. JP 2011516081-A/4: THROMBIN ACTIVATOR COMPOSITIONS AND METHODS OF MAKING
AND USING THE SAME 1,422 bp linear DNA HV932640.1 GI: 415676392
288. JP 2011516081-A/3: THROMBIN ACTIVATOR COMPOSITIONS AND METHODS OF MAKING
AND USING THE SAME 1,404 bp linear DNA HV932639.1 GI: 415676389
289. JP 2011516081-A/2: THROMBIN ACTIVATOR COMPOSITIONS AND METHODS OF MAKING
AND USING THE SAME 1,869 bp linear DNA HV932638.1 GI: 415676386
290. JP 2011516081-A/1: THROMBIN ACTIVATOR COMPOSITIONS AND METHODS OF MAKING
AND USING THE SAME 1,869 bp linear DNA HV932637.1 GI: 415676383
291. Chlamydia trachomatis strain 404 metalloprotease (ispH) and hypothetical protein (CT860) genes,
complete cds 2,468 bp linear DNA JQ066538.1 GI: 407651452
292. Chlamydia trachomatis strain 440 metalloprotease (ispH) and hypothetical protein (CT860) genes,
complete cds 2,468 bp linear DNA JQ066537.1 GI: 407651449
293. Chlamydia trachomatis strain UW31 metalloprotease (ispH) and hypothetical protein (CT860) genes,
complete cds 2,468 bp linear DNA JQ066536.1 GI: 407651446
294. Chlamydia trachomatis strain UW36 metalloprotease (ispH) and hypothetical protein (CT860) genes,
complete cds 2,468 bp linear DNA JQ066535.1 GI: 407651443
295. Chlamydia trachomatis strain UW12 metalloprotease (ispH) and hypothetical protein (CT860) genes,
complete cds 2,468 bp linear DNA JQ066534.1 GI: 407651440
296. Chlamydia trachomatis strain UW43 metalloprotease (ispH) and hypothetical protein (CT860) genes,
complete cds 2,468 bp linear DNA JQ066533.1 GI: 407651437
297. Chlamydia trachomatis strain UW57 metalloprotease (ispH) and hypothetical protein (CT860) genes,
complete cds 2,468 bp linear DNA JQ066532.1 GI: 407651434
298. Chlamydia trachomatis strain IC-Cal3 metalloprotease (ispH) and hypothetical protein (CT860) genes,
complete cds 2,468 bp linear DNA JQ066531.1 GI: 407651431
299. Chlamydia trachomatis strain Bour metalloprotease (ispH) and hypothetical protein (CT860) genes,
complete cds 2,468 bp linear DNA JQ066530.1 GI: 407651428
300. Chlamydia trachomatis strain TW3 metalloprotease (ispH) and hypothetical protein (CT860) genes,
complete cds 2,468 bp linear DNA JQ066529.1 GI: 407651425
301. Chlamydia trachomatis strain Apache2 metalloprotease (ispH) and hypothetical protein (CT860) genes,
complete cds 2,468 bp linear DNA JQ066528.1 GI: 407651422
302. Solanum lycopersicum cultivar Ailsa Craig lutescent 2 gene, complete cds 9,192 bp linear DNA
JQ683149.1 GI: 395484830
303. Legionella pneumophila 2300/99 zinc metalloprotease (proA) gene, complete cds 1,632 bp linear DNA
EU221245.1 GI: 165968099
304. Bacillus anthracis strain 34F2 lethal factor (lef) gene, complete cds 2,430 bp linear DNA JQ798176.1
GI: 390132838
305. Vibrio vulnificus vvsA, vvsB genes for alkaline serine protease, hypothetical protein, complete cds,
strain: NCIMB 2137 3,210 bp linear DNA AB509375.1 GI: 244538827
306. Exiguobacterium undae gene for protease, complete cds, strain: Su-1 2,119 bp linear DNA AB669473.1
GI: 386685043
307. Xenorhabdus nematophila strain Az157 secreted alkaline metalloprotease (prtA) gene, complete cds
1,437 bp linear DNA GU293162.1 GI: 310894075
308. Xenorhabdus nematophila strain Az155 secreted alkaline metalloprotease (prtA) gene, complete cds
1,437 bp linear DNA GU293161.1 GI: 310894073
309. Xenorhabdus nematophila strain Az154 secreted alkaline metalloprotease (prtA) gene, complete cds
1,437 bp linear DNA GU293160.1 GI: 310894071
310. Xenorhabdus nematophila strain Az152 secreted alkaline metalloprotease (prtA) gene, complete cds
1,437 bp linear DNA GU293159.1 GI: 310894069
311. Xenorhabdus nematophila strain Az150 secreted alkaline metalloprotease (prtA) gene, complete cds
1,437 bp linear DNA GU293158.1 GI: 310894067
312. Xenorhabdus nematophila strain Az149 secreted alkaline metalloprotease (prtA) gene, complete cds
1,437 bp linear DNA GU293157.1 GI: 310894065
313. Xenorhabdus nematophila strain Az143 secreted alkaline metalloprotease (prtA) gene, complete cds
1,437 bp linear DNA GU293156.1 GI: 310894063
314. Xenorhabdus nematophila strain Az20 secreted alkaline metalloprotease (prtA) gene, complete cds
1,437 bp linear DNA GU293155.1 GI: 310894061
315. Xenorhabdus nematophila strain R1 secreted alkaline metalloprotease (prtA) gene, complete cds 1,437
bp linear DNA GU293154.1 GI: 310894059
316. Xenorhabdus nematophila strain Bcn14 secreted alkaline metalloprotease (prtA) gene, complete cds
1,437 bp linear DNA GU293153.1 GI: 310894057
317. Xenorhabdus nematophila strain Caba02 secreted alkaline metalloprotease (prtA) gene, complete cds
1,437 bp linear DNA GU293152.1 GI: 310894055
318. Oryctolagus cuniculus matrix metalloproteinase (MMP9) gene, exons 1-3 1,862 bp linear DNA
L36050.1 GI: 535714
319. Plasmid pSS20 vector system DNA encoding beta-galactosidase, 3′ end 294 bp linear DNA M18897.1
GI: 209166
320. Human collagenase gene, 5′ end 698 bp linear DNA M16567.1 GI: 180668
321. Human type-4 collagenase (CLG4) preproenzyme gene, exon 1 451 bp linear DNA M33789.1
GI: 180600
322. Streptomyces lividans metalloprotease (prt) gene, complete cds 1,899 bp linear DNA M89476.1
GI: 153411
323. E.chrysanthemi protease B (prtB) gene, complete cds, and protease C (prtC) gene, 5′ end 1,871 bp linear
DNA J04736.1 GI: 148493
324. Streptococcus pneumoniae zinc metalloprotease C (zmpC) gene, complete cds 5,571 bp linear DNA
JQ320497.1 GI: 37782341
325. Mycobacterium bolletii strain CIP108541 putative metalloprotease gene, complete cds 513 bp linear
DNA HQ661997.1 GI: 336441267
326. Pseudomonas fluorescens strain Bk3 extracellular metalloprotease AprX gene, complete cds 1,434 bp
linear DNA GQ862303.1 GI: 291191918
327. Proteus mirabilis strain Pm7 metalloprotease zapA (zapA) gene, complete cds 1,476 bp linear DNA
HM217133.1 GI: 299818420
328. Sequence 29 from Patent WO2011133538 2,387 bp linear DNA JA662264.1 GI: 357993029
329. Sequence 28 from Patent WO2011133538 1,973 bp linear DNA JA662263.1 GI: 357993028
330. Sequence 29 from Patent WO2011133539 2,387 bp linear DNA JA662191.1 GI: 357992959
331. Sequence 28 from Patent WO2011133539 1,973 bp linear DNA JA662190.1 GI: 357992958
332. Bacillus amyloliquefaciens strain A50 extracellular metalloprotease gene, complete cds 972 bp linear
DNA GU992366.1 GI: 294986359
333. Sequence 5 from U.S. Pat. No. 8,012,471 2,262 bp linear DNA GY439356.1 GI: 348620311
334. Sequence 3 from U.S. Pat. No. 8,012,471 2,340 bp linear DNA GY439355.1 GI: 348620310
335. Sequence 1 from U.S. Pat. No. 8,012,471 2,076 bp linear DNA GY439354.1 GI: 34862030
336. Mycobacterium immunogenum strain CIP106684 putative metalloprotease gene, complete cds 513 bp
linear DNA HQ661999.1 GI: 336441271
337. Mycobacterium massiliense strain CCUG48898 putative metalloprotease gene, complete cds 513 bp
linear DNA HQ662000.1 GI: 336441273
338. Mycobacterium chelonae strain ATCC 35752 putative metalloprotease gene, complete cds 513 bp linear
DNAHQ661998.1 GI: 336441269
339. Mycobacterium franklinii strain CV002 putative metalloprotease gene, complete cds 513 bp linear DNA
HQ661996.1 GI: 336441265
340. Mycobacterium abscessus strain CIP104536 putative metalloprotease gene, complete cds 513 bp linear
DNA HQ661995.1 GI: 336441263
341. Brugia malayi astacin metalloprotease (nas-36) gene, complete cds 4,825 bp linear DNA FJ812520.1
GI: 270209734
342. Haemonchus contortus astacin metalloprotease (nas-36) gene, complete cds 9,663 bp linear DNA
FJ812519.1 GI: 270209732
343. Vibrio coralliilyticus strain LMG 23696 zinc-metalloprotease (vcpA) gene, complete cds 1,824 bp linear
DNA GQ452012.1 GI: 262262685
344. Albugo laibachii Nc14, genomic contig CONTIG_1178_NC14_v4_3148_231 3,148 bp linear DNA
FR824909.1 GI: 325193978
345. JP 2000511429-A/6: Fungal metalloprotease gene 1,760 bp linear DNA HV195760.1 GI: 34030182
346. JP 2000511429-A/5: Fungal metalloprotease gene 1,920 bp linear DNA HV195759.1 GI: 340301823
347. JP 2000511429-A/4: Fungal metalloprotease gene 1,062 bp linear DNA HV195758.1 GI: 340301822
348. JP 2000511429-A/3: Fungal metalloprotease gene 1,186 bp linear DNA HV195757.1 GI: 340301821
349. JP 2000511429-A/2: Fungal metalloprotease gene 1,047 bp linear DNA HV195756.1 GI: 340301820
350. JP 2000511429-A/1: Fungal metalloprotease gene 1,173 bp linear DNA HV195755.1 GI: 34030181
351. JP 2006204304-A/2: HOST CELL EXPRESSING REDUCED LEVELS OF A METALLOPROTEASE
AND METHODS USING THE HOST CELL IN PROTEIN PRODUCTION 747 bp linear DNA
HV188501.1 GI: 340174872
352. JP 2006204304-A/1: HOST CELL EXPRESSING REDUCED LEVELS OF A METALLOPROTEASE
AND METHODS USING THE HOST CELL IN PROTEIN PRODUCTION 2,052 bp linear DNA
HV188500.1 GI: 340174871
353. JP 2009511072-A/10: Use and Production of Storage-Stable Neutral Metalloprotease 1,566 bp linear
DNA HV234235.1 GI: 340086610
354. JP 2009511072-A/2: Use and Production of Storage-Stable Neutral Metalloprotease 1,578 bp linear
DNA HV234227.1 GI: 340086602
355. JP 2009511072-A/1: Use and Production of Storage-Stable Neutral Metalloprotease 713 bp linear DNA
HV234226.1 GI: 340086601
356. Grimontia hollisae gene for collagenase, complete cds, strain: 1706B 2,304 bp linear DNA AB600550.1
GI: 336088230
357. Sequence 9 from U.S. Pat. No. 7,867,747 853 bp linear DNA GX902550.1 GI: 330454570
358. Sequence 8 from U.S. Pat. No. 7,867,747 2,449 bp linear DNA GX902549.1 GI: 330454569
359. Sequence 5 from U.S. Pat. No. 7,867,747 2,427 bp linear DNA GX902546.1 GI: 330454566
360. Sequence 4 from U.S. Pat. No. 7,867,747 546 bp linear DNA GX902545.1 GI: 330454565
361. Sequence 3 from U.S. Pat. No. 7,867,747 542 bp linear DNA GX902544.1 GI: 330454564
362. Sequence 1 from U.S. Pat. No. 7,867,747 1,080 bp linear DNA GX902543.1 GI: 330454563
363. Leishmania donovani donovani strain MHOM/IN/1983/AG83 gp63 gene, complete cds 1,800 bp linear
DNA GQ301544.1 GI: 253400289
364. Photorhabdus luminescens subsp. laumondii strain Az148 secreted alkaline metalloprotease (prtA) gene,
complete cds 1,455 bp linear DNA AY928068.1 GI: 62754188
365. Photorhabdus luminescens subsp. laumondii strain Az144 secreted alkaline metalloprotease (prtA) gene,
complete cds 1,455 bp linear DNA AY928067.1 GI: 62754186
366. Photorhabdus luminescens subsp. laumondii strain Az140 secreted alkaline metalloprotease (prtA) gene,
complete cds 1,455 bp linear DNA AY928066.1 GI: 62754184
367. Photorhabdus luminescens subsp. laumondii strain Az39 secreted alkaline metalloprotease (prtA) gene,
complete cds 1,455 bp linear DNA AY928065.1 GI: 62754182
368. Photorhabdus luminescens subsp. laumondii strain Az36 secreted alkaline metalloprotease (prtA) gene,
complete cds 1,455 bp linear DNA AY928064.1 GI: 62754180
369. Photorhabdus luminescens subsp. laumondii strain Az35 secreted alkaline metalloprotease (prtA) gene,
complete cds 1,455 bp linear DNA AY928063.1 GI: 62754178
370. Photorhabdus luminescens subsp. laumondii strain Az34 secreted alkaline metalloprotease (prtA) gene,
complete cds 1,455 bp linear DNA AY928062.1 GI: 62754176
371. Photorhabdus luminescens subsp. laumondii strain Az33 secreted alkaline metalloprotease (prtA) gene,
complete cds 1,455 bp linear DNA AY928061.1 GI: 62754174
372. Photorhabdus luminescens subsp. laumondii strain Az3 1 secreted alkaline metalloprotease (prtA) gene,
complete cds 1,455 bp linear DNA AY928060.1 GI: 62754172
373. Photorhabdus luminescens subsp. laumondii strain Az30 secreted alkaline metalloprotease (prtA) gene,
complete cds 1,455 bp linear DNA AY928059.1 GI: 62754170
374. Photorhabdus luminescens subsp. laumondii strain Az28 secreted alkaline metalloprotease (prtA) gene,
complete cds 1,455 bp linear DNA AY928058.1 GI: 62754168
375. Photorhabdus luminescens subsp. laumondii strain HP88 secreted alkaline metalloprotease (prtA) gene,
complete cds 1,455 bp linear DNA AY928057.1 GI: 62754166
376. Photorhabdus temperata strain C1 secreted alkaline metalloprotease (prtA) gene, complete cds 1,452 bp
linear DNA AY928056.1 GI: 62754164
377. Flavobacterium psychrophilum pth, fpp1, fpp2 and ribf genes, strain THC02/90 8,220 bp linear DNA
FR667216.1 GI: 300806898
378. JP 2010248195-A/4: METALLOPROTEASE ACTIVATION OF MYOSTATIN, AND METHODS OF
MODULATING MYOSTATIN ACTIVITY 1,125 bp linear DNA FW576587.1 GI: 324302836
379. JP 2010248195-A/3: METALLOPROTEASE ACTIVATION OF MYOSTATIN, AND METHODS OF
MODULATING MYOSTATIN ACTIVITY 1,128 bp linear DNA FW576586.1 GI: 324302835
380. JP 2010248195-A/2: METALLOPROTEASE ACTIVATION OF MYOSTATIN, AND METHODS OF
MODULATING MYOSTATIN ACTIVITY 1,128 bp linear DNA FW576585.1 GI: 324302834
381. JP 2010248195-A/1: METALLOPROTEASE ACTIVATION OF MYOSTATIN, AND METHODS OF
MODULATING MYOSTATIN ACTIVITY 2,743 bp linear DNA FW576584.1 GI: 324302833
382. JP 2010263880-A/5: Novel collagenase gene from a microorganism 2,304 bp linear DNA FW576122.1
GI: 324302180
383. JP 2010263880-A/2: Novel collagenase gene from a microorganism 261 bp linear DNA FW576119.1
GI: 324302177
384. JP 2010263880-A/1: Novel collagenase gene from a microorganism 2,040 bp linear DNA FW576118.1
GI: 324302176
385. Legionella pneumophila strain ATCC BAA-74 glycerophospholipid: cholesterol acyltransferase (plaC)
gene, complete cds 1,383 bp linear DNA AY745197.1 GI: 58200460
386. Burkholderia pseudomallei extracellular zinc metalloprotease precursor (zmpA) gene, complete cds
1,698 bp linear DNA AY143551.1 GI: 33303603
387. Flavobacterium sp. YS-80-122 alkaline metalloprotease (lupA) gene, complete cds 1,482 bp linear DNA
GU084389.1 GI: 262093148
388. Bacillus thuringiensis serovar israelensis camelysin (calY) gene, complete cds 983 bp linear DNA
EU254516.1 GI: 162135981
389. Vibrio vulnificus strain E86 zinc metalloprotease (vvp) gene, complete cds 1,830 bp linear DNA
DQ923325.1 GI: 115353966
390. Bacillus thuringiensis serovar kurstaki camelysin (calY) gene, complete cds 600 bp linear DNA
EU604076.1 GI: 183604402
391. Pseudomonas aeruginosa elastase precursor (lasB) gene, complete cds 1,560 bp linear DNA
DQ150629.1 GI: 73612141
392. Bacillus intermedius strain 3-19 metal-dependent phosphohydrolase (ywfO), hypothetical protein
(ywfA), secreted metalloprotease (mprBi), membrane bound metalloprotease (pmbBi), hypothetical protein
(ywhD), and penicillin-binding protein (ywhE) genes, complete cds 5,896 bp linear DNA EU678894.2
GI: 215398146
393. Vibrio parahaemolyticus collagenase gene, complete cds 2,445 bp linear DNA AF326572.1
GI: 12584927
394. Sequence 3548 from Patent WO2010102262 5,360 bp linear DNA HH825958.1 GI: 308381813
395. Sequence 3474 from Patent WO2010102262 6,630 bp linear DNA HH825884.1 GI: 308381812
396. Sequence 3466 from Patent WO2010102262 5,369 bp linear DNA HH825876.1 GI: 308381811
397. Sequence 776 from Patent WO2010102262 2,480 bp linear DNA HH823186.1 GI: 308381810
398. Sequence 773 from Patent WO2010102262 1,647 bp linear DNA HH823183.1 GI: 308381809
399. Sequence 770 from Patent WO2010102262 1,246 bp linear DNA HH823180.1 GI: 308381808
400. Sequence 767 from Patent WO2010102262 1,919 bp linear DNA HH823177.1 GI: 308381807
401. Sequence 764 from Patent WO2010102262 3,262 bp linear DNA HH823174.1 GI: 308381806
402. Sequence 761 from Patent WO2010102262 1,674 bp linear DNA HH823171.1 GI: 308381805
403. Sequence 758 from Patent WO2010102262 3,565 bp linear DNA HH823168.1 GI: 308381804
404. Sequence 755 from Patent WO2010102262 4,344 bp linear DNA HH823165.1 GI: 308381803
405. Sequence 752 from Patent WO2010102262 2,438 bp linear DNA HH823162.1 GI: 308381802
406. Sequence 749 from Patent WO2010102262 6,347 bp linear DNA HH823159.1 GI: 308381801
407. Sequence 746 from Patent WO2010102262 4,456 bp linear DNA HH823156.1 GI: 308381800
408. Sequence 743 from Patent WO2010102262 3,558 bp linear DNA HH823153.1 GI: 308381799
409. Sequence 740 from Patent WO2010102262 1,825 bp linear DNA HH823150.1 GI: 308381798
410. Sequence 737 from Patent WO2010102262 998 bp linear DNA HH823147.1 GI: 308381797
411. Sequence 734 from Patent WO2010102262 1,147 bp linear DNA HH823144.1 GI: 308381796
412. Sequence 731 from Patent WO2010102262 2,247 bp linear DNA HH823141.1 GI: 308381795
413. Sequence 728 from Patent WO2010102262 1,743 bp linear DNA HH823138.1 GI: 308381794
414. Sequence 725 from Patent WO2010102262 1,828 bp linear DNA HH823135.1 GI: 308381793
415. Sequence 722 from Patent WO2010102262 2,387 bp linear DNA HH823132.1 GI: 308381792
416. Sequence 719 from Patent WO20101022623,546 bp linear DNA HH823129.1 GI: 308381791
417. Sequence 716 from Patent WO2010102262 1,609 bp linear DNA HH823126.1 GI: 308381790
418. Sequence 713 from Patent WO2010102262 2,722 bp linear DNA HH823123.1 GI: 308381789
419. Sequence 710 from Patent WO2010102262 2,223 bp linear DNA HH823120.1 GI: 308381788
420. Sequence 708 from Patent WO2010102262 1,973 bp linear DNA HH823118.1 GI: 308381787
421. Sequence 706 from Patent WO2010102262 1,353 bp linear DNA HH823116.1 GI: 308381783
422. Vibrio aestuarianus metalloprotease precursor (VAM) gene, complete cds 1,852 bp linear DNA
AY605667.1 GI: 78125891
423. Microsporum canis metalloprotease MEP4 (mep4) gene, complete cds 2,184 bp linear DNA
AY283573.1 GI: 33520310
424. Microsporum canis metalloprotease MEP5 (mep5) gene, complete cds 2,191 bp linear DNA
AY283570.1 GI: 33520304
425. A Collagenase with an affinity tag, and its preparation method 3,105 bp linear DNA FW351359.1
GI: 305395600
426. A Collagenase with an affinity tag, and its preparation method 3,396 bp linear DNA FW351358.1
GI: 305395599
427. Trichophyton equinum metalloprotease 3 (Mep3) gene, complete cds 2,395 bp linear DNA FJ348247.1
GI: 219816469
428. Trichophyton tonsurans metalloprotease 1 (Mep1) gene, complete cds 2,321 bp linear DNA FJ348242.1
GI: 219816459
429. Trichophyton tonsurans metalloprotease 4 (MEP4) gene, complete cds 2,335 bp linear DNA FJ267697.1
GI: 210076630
430. Trichophyton tonsurans metalloprotease 2 (MEP2) gene, complete cds 2,223 bp linear DNA FJ267696.1
GI: 210076628
431. Trichophyton tonsurans metalloprotease 3 gene, complete cds 2,443 bp linear DNA FJ349344.1
GI: 209974064
432. Homo sapiens aggrecanase 1 (ADAMTS4) gene, complete cds 10,766 bp linear DNA AY044847.1
GI: 15667234
433. Vibrio harveyi VhpA (vhpA) and VhpB (vhpB) genes, complete cds 4,400 bp linear DNA AY630354.1
GI: 49823287
434. Porphyromonas gingivalis strain 47A-1 collagenase (prtC) gene, complete cds 1,005 bp linear DNA
AY633706.1 GI: 48857026
435. Actinobacillus pleuropneumoniae aminopeptidase gene, complete cds 2,610 bp linear DNA
AY545035.1 GI: 44981978
436. Vibrio vulnificus metalloprotease gene, complete cds 2,660 bp linear DNA U50548.1 GI: 2114338
437. Vibrio vulnificus zinc metalloprotease (vvp) gene, complete cds 2,407 bp linear DNA U48780.1
GI: 1794193
438. Saccharomyces cerevisiae zinc metallo-protease (STE24) gene, complete cds 1,706 bp linear DNA
U77137.1 GI: 1679740
439. Bacteroides fragilis enterotoxin gene (bftP), complete cds 1,700 bp linear DNA U67735.1 GI: 1527173
440. Flavobacterium meningosepticum aspartyl endopeptidase gene, complete cds 1,332 bp linear DNA
L37784.1 GI: 825443
441. Streptomyces lividans aminopeptidase N gene, complete cds 2,849 bp linear DNA L23172.1 GI: 487884
442. Aspergillus fumigatus metalloprotease exons 1-5, complete cds 2,692 bp linear DNA L29566.1
GI: 461376
443. Streptococcus sanguis IgA1 protease gene, complete cds 5,826 bp linear DNA L29504.1 GI: 460338
444. prtD = protease SM transporter, prtE = protease SM transporter [Serratia marcescens, 365, Genomic, 3368
nt] 3,368 bp linear DNA S67013.1 GI: 452927
445. Serratia marcescens 11 kDa protease inhibitor gene, complete cds 536 bp linear DNA L09107.1
GI: 152842
446. Porphyromonas gingivalis prtC gene expressing collagenase activity, complete cds 1,197 bp linear DNA
M60404.1 GI: 150847
447. L.pneumophila zinc metalloprotease (pro A) gene, complete cds 1,766 bp linear DNA M31884.1
GI: 149695
448. E.chrysanthemi metalloprotease C (prtC) gene, complete cds 1,653 bp linear DNA M59229.1
GI: 148491
449. E.chrysanthemi metalloprotease C (prtC) gene, complete cds 2,164 bp linear DNA M37390.1
GI: 148489
450. Bacillus subtilis extracellular metalloprotease (mpr) gene, complete cds 998 bp linear DNA L10505.1
GI: 143209
451. Listonella anguillarum strain M93Sm metalloprotease (empA) gene, complete cds 2,220 bp linear DNA
AY428808.1 GI: 40557597
452. Leishmania amazonensis ecto-metalloproteinase precursor (gp63) gene, complete cds 2,046 bp linear
DNA L46798.1 GI: 1100212
453. Vibrio anguillarum metalloproteinase (empA) gene, complete cds 2,256 bp linear DNA L02528.1
GI: 155164
454. Sequence 7 from Patent EP2192129 1,125 bp linear DNA HC889212.1 GI: 298216575
455. Sequence 5 from Patent EP2192129 1,128 bp linear DNA HC889210.1 GI: 298216573
456. Sequence 3 from Patent EP2192129 1,128 bp linear DNA HC889208.1 GI: 298216571
457. Sequence 1 from Patent EP2192129 2,743 bp linear DNA HC889206.1 GI: 298216569
458. Arthroderma otae MEP3 gene for metalloprotease 3, complete cds 3,244 bp linear DNA AB125268.1
GI: 164456606
459. Arthroderma otae mcmp1 gene for metalloprotease1, complete cds 3,244 bp linear DNA AB097684.1
GI: 164456604
460. Microsporum canis mep2 gene for metalloprotease (MEP2), exons 1-2 1,957 bp linear DNA
AJ490185.1 GI: 24412836
461. Microsporum canis mep1 gene for metalloprotease (MEP1), exons 1-5 2,172 bp linear DNA
AJ490184.1 GI: 24412834
462. Microsporum canis mep3 gene for metalloprotease (MEP3), exons 1-5 2,186 bp linear DNA
AJ490183.1 GI: 24412832
463. Burkholderia cepacia extracellular zinc metalloprotease PSCP precursor (zmpA) gene, complete cds
1,698 bp linear DNA AY143552.1 GI: 33303605
464. Bacillus thuringiensis regulator SinR (sinR), regulator SinI (sinI), and immune inhibitor A genes,
complete cds 3,759 bp linear DNA AF287346.1 GI: 9858107
465. Pseudomonas fluorescens PrtB (prtB) and lipase (lipA) genes, complete cds 5,423 bp linear DNA
AF216702.1 GI: 8895499
466. Pseudomonas fluorescens genomic sequence 1,467 bp linear DNA AF216701.1 GI: 8895498
467. Pseudomonas fluorescens metalloprotease (aprX) gene, complete cds 1,941 bp linear DNA AF216700.1
GI: 8895496
468. Human collagenase-3 gene, promoter region 1,624 bp linear DNA U52692.1 GI: 1923215
469. Erwinia carotovora subsp. carotovora metalloprotease (prtW) and protease inhibitor (inh) genes,
complete cds 3,165 bp linear DNA AF141295.2 GI: 15149007
470. Brugia malayi astacin metalloprotease (nas-35) gene, complete cds, alternatively spliced 8,230 bp linear
DNA FJ812518.1 GI: 270209729
471. Brugia malayi astacin metalloprotease (nas-38) gene, complete cds 9,111 bp linear DNA FJ812522.1
GI: 270209738
472. Listeria monocytogenes strain XFL0605 Mp1 (mp1) gene, complete cds 1,533 bp linear DNA
EF183453.1 GI: 124495034
473. Enterococcus faecalis GM gelatinase (gelE) gene, complete cds 1,836 bp linear DNA EF105504.1
GI: 118722726
474. Listonella anguillarum VanT (vanT) gene, complete cds 1,759 bp linear DNA AF457643.2
GI: 40850677
475. Streptomyces exfoliatus LieB (lieB) gene, complete cds 3,938 bp linear DNA AY335439.1
GI: 34499903
476. Arthroderma benhamiae metalloprotease MEP4 (mep4) gene, complete cds 2,185 bp linear DNA
AY283576.1 GI: 33520316
477. Arthroderma benhamiae metalloprotease MEP2 (mep2) gene, complete cds 1,964 bp linear DNA
AY283575.1 GI: 33520314
478. Arthroderma benhamiae metalloprotease MEP3 (mep3) gene, complete cds 2,241 bp linear DNA
AY283574.1 GI: 33520312
479. Arthroderma benhamiae metalloprotease MEP1 (mep1) gene, complete cds 2,213 bp linear DNA
AY283572.1 GI: 33520308
480. Arthroderma benhamiae metalloprotease MEP5 (mep5) gene, complete cds 2,226 bp linear DNA
AY283571.1 GI: 33520306
481. Trichophyton rubrum metalloprotease MEP3 (mep3) gene, complete cds 2,241 bp linear DNA
AY283569.1 GI: 33520302
482. Coccidioides posadasii metalloprotease 1 precursor (MEP1) gene, complete cds 3,417 bp linear DNA
AF500214.1 GI: 33323430
483. Pseudomonas fluorescens strain A506 extracellular alkaline metalloprotease (aprX) gene, complete cds
2,431 bp linear DNA AY298902.1 GI: 31335336
484. Trypanosoma cruzi GP63 group II protein (GP63-II) gene, complete cds 1,760 bp linear DNA
AY266318.1 GI: 31322789
485. Trypanosoma cruzi GP63 group I member b protein (GP63-Ib) gene, complete cds 1,896 bp linear DNA
AY266317.1 GI: 31322787
486. Trypanosoma cruzi GP63 group I member a protein (GP63-Ia) gene, complete cds 2,072 bp linear DNA
AY2663 16.1 GI: 31322785
487. Pseudomonas fluorescens alkaline protease, protease inhibitor, zinc-protease transporter (aprD), zinc-
protease transporter (aprE), and zinc-protease transporter (aprF) genes, complete cds 7,334 bp linear DNA
AF004848.2 GI: 30410774
488. Trypanosoma brucei major surface protease-like protein C (MSP-C) gene, complete cds 1,776 bp linear
DNA AY230807.1 GI: 29602511
489. Chromobacterium violaceum class 4 metalloprotease gene, complete cds 2,454 bp linear DNA
AY161300.1 GI: 26419738
490. Trichophyton rubrum putative secreted metalloprotease 4 (MEP4) gene, complete cds 2,670 bp linear
DNA AF407191.1 GI: 22652156
491. Trichophyton rubrum putative secreted metalloprotease 5 (MEP5) gene, complete cds 2,261 bp linear
DNA AF407189.1 GI: 22652152
492. Trichophyton rubrum putative secreted metalloprotease 2 (MEP2) gene, complete cds 2,192 bp linear
DNA AF407187.1 GI: 22652148
493. Trichophyton rubrum putative secreted metalloprotease 1 (MEP1) gene, complete cds 2,265 bp linear
DNA AF407185.1 GI: 22652144
494. Renibacterium salmoninarum isolate 980036-150 hemolysin (hly) gene, complete cds 1,647 bp linear
DNA AF428067.1 GI: 21321398
495. Helicobacter pylori metalloprotease gene, complete cds 621 bp linear DNA AF380136.1 GI: 21310088
496. Vibrio harveyi zinc metalloprotease Pap6 gene, complete cds 2,034 bp linear DNA AF508306.1
GI: 21070335
497. Synthetic construct Clostridium botulinum neurotoxin type A LHn fragment gene, complete cds 2,616
bp linear DNA AF464912.1 GI: 18251975
498. Myxococcus xanthus putative oxidoreductase, matrix-associated zinc metalloprotease FibA (fibA), and
putative phosphoesterase genes, complete cds 5,690 bp linear DNA AF457462.1 GI: 18140051
499. Bacillus thuringiensis zinc-metalloprotease (inhA2) gene, complete cds 3,087 bp linear DNA
AF421888.1 GI: 16226176
500. Choristoneura fumiferana granulovirus enhancin gene, complete cds 2,706 bp linear DNA AF319939.1
GI: 11276052
501. Vibrio vulnificus VvpR (vvpR) gene, complete cds 1,300 bp linear DNA AY007308.1 GI: 10180983
502. Vibrio vulnificus strain CKUH metalloprotease precursor (vvpE) gene, complete cds 1,835 bp linear
DNA FJ864304.1 GI: 227437414
503. Escherichia coli strain TW14359 putative collagenase (ECs2039) gene, complete cds 2,004 bp linear
DNA EU901274.1 GI: 209771015
504. Escherichia coli strain TB182A putative collagenase (ECs2039) gene, complete cds 2,004 bp linear
DNA EU901273.1 GI: 209771013
505. Escherichia coli strain 87-14 putative collagenase (ECs2039) gene, complete cds 2,004 bp linear DNA
EU901272.1 GI: 209771011
506. Escherichia coli strain 86-24 putative collagenase (ECs2039) gene, complete cds 2,004 bp linear DNA
EU901271.1 GI: 209771009
507. Escherichia coli strain 493/89 putative collagenase (ECs2039) gene, complete cds 2,004 bp linear DNA
EU901270.1 GI: 209771007
508. Escherichia coli strain TW14359 putative collagenase (ECs4039) gene, complete cds 996 bp linear
DNA EU894974.1 GI: 209758445
509. Escherichia coli strain TB182A putative collagenase (ECs4039) gene, complete cds 996 bp linear DNA
EU894973.1 GI: 209758443
510. Escherichia coli strain 87-14 putative collagenase (ECs4039) gene, complete cds 996 bp linear DNA
EU894972.1 GI: 209758441
511. Escherichia coli strain 86-24 putative collagenase (ECs4039) gene, complete cds 996 bp linear DNA
EU894971.1 GI: 209758439
512. Escherichia coli strain 493/89 putative collagenase (ECs4039) gene, complete cds 996 bp linear DNA
EU894970.1 GI: 209758437
513. Staphylococcus chromogenes metalloprotease (scp) gene, complete cds 1,506 bp linear DNA
AF218055.1 GI: 6942069
514. Streptococcus pneumoniae putative response regulator (zmpR), putative histidine kinase (zmpS), and
putative zinc metalloprotease (zmpB) genes, complete cds 8,847 bp linear DNA AF221126.1 GI: 6911254
515. Aeromonas hydrophila elastase (ahpB) gene, complete cds 1,929 bp linear DNA AF193422.1
GI: 6319149
516. Bacteroides fragilis 419 putative metalloprotease II (t3) and BFT-3 (bft-3) genes, complete cds; and
unknown gene 6,096 bp linear DNA AF103902.1 GI: 4927635
517. Bacteroides fragilis metalloprotease enterotoxin gene, complete cds 1,194 bp linear DNA AF081785.2
GI: 4836836
518. Vibrio cholerae collagenase (vcc) gene, complete cds 2,934 bp linear DNA AF080248.1 GI: 3925384
519. Vibrio vulnificus metalloprotease (vvp) gene, complete cds 2,437 bp linear DNA AF102028.1
GI: 3851712
520. Proteus mirabilis metalloprotease operon, complete sequence 9,345 bp linear DNA AF064762.1
GI: 3493594
521. Bacteroides fragilis strain VPI 13784 putative metalloprotease II and fragilysin genes, complete cds; and
unknown gene 6,832 bp linear DNA AF038459.1 GI: 3046919
522. Lactobacillus helveticus endopeptidase O (pepO) gene, complete cds 2,691 bp linear DNA AF019410.1
GI: 2738891
523. Proteus mirabilis metalloprotease gene, complete cds 1,940 bp linear DNA U25950.1 GI: 829593
524. Tannerella forsythia strain ATCC 43037 karilysin protease gene, complete cds 1,563 bp linear DNA
GQ856797.1 GI: 259490993
525. Escherichia coli stcE gene for zinc metalloprotease, strain FHI_K9, serovar O103 3,031 bp linear DNA
AM901563.1 GI: 164513986
526. Low temperature active collagenase 1,536 bp linear DNA DM473818.1 GI: 284405589
527. Vibrio vulnificus gene for metalloprotease, complete cds, strain: 95-8-161 1,830 bp linear DNA
AB540652.1 GI: 283806442
528. Vibrio vulnificus gene for metalloprotease, complete cds, strain: CECT 5198 1,830 bp linear DNA
AB540651.1 GI: 283806440
529. Vibrio vulnificus gene for metalloprotease, complete cds, strain: CECT 5343 1,830 bp linear DNA
AB540650.1 GI: 283806438
530. Pseudomonas fluorescens strain TSS extracellular metalloprotease (aprX) gene, complete cds 1,434 bp
linear DNA FJ687263.1 GI: 251826355
531. Bacillus thuringiensis serovar thuringiensis strain 407 InhA3 (inhA3) gene, complete cds 3,836 bp linear
DNA FJ717416.1 GI: 224593232
532. Vibrio tubiashii strain X00-12-1(RE98) VthB (vthB) and VthA (vthA) genes, complete cds 1,924 bp
linear DNA FJ593884.1 GI: 222107822
533. Sus scrofa matrix metallopeptidase 10 (MMP10) gene, complete cds 7,030 bp linear DNA EU722908.1
GI: 199652493
534. Sus scrofa matrix metallopeptidase 1 (MMP1) gene, complete cds 8,460 bp linear DNA EU722906.1
GI: 199652448
535. Listonella anguillarum PrtV (prtV) and LlpB (llpB) genes, complete cds 4,064 bp linear DNA
GQ118991.1 GI: 239509288
536. Enterobacter sakazakii zinc metalloprotease (zpx) gene, complete cds 1,026 bp linear DNA EF061082.1
GI: 117671277
537. Francisella tularensis subsp. novicida PepO (pepO) gene, complete cds 2,064 bp linear DNA
DQ230367.1 GI: 82452886
538. Listeria monocytogenes strain A23 metalloprotease gene, complete cds 1,533 bp linear DNA
FJ932479.1 GI: 272983537
539. Vibrio tubiashii VtpB (vtpB) gene, complete cds 3,042 bp linear DNA GQ121132.1 GI: 239809226
540. Burkholderia cenocepacia strain LMG 18832 extracellular zinc metalloprotease precursor (zmpA) gene,
complete cds 1,698 bp linear DNA DQ069250.1 GI: 71068369
541. Burkholderia cenocepacia strain LMG 16654 extracellular zinc metalloprotease precursor (zmpA) gene,
complete cds 1,698 bp linear DNA DQ069249.1 GI: 71068367
542. Burkholderia cenocepacia strain LMG 18827 extracellular zinc metalloprotease precursor (zmpA) gene,
complete cds 1,698 bp linear DNA DQ069248.1 GI: 71068365
543. Burkholderia cenocepacia strain K56-2 extracellular zinc metalloprotease precursor (zmpA) gene,
complete cds 1,698 bp linear DNA DQ069247.1 GI: 71068363
544. Streptococcus mitis strain SK564 IgA1 protease (iga) gene, complete cds 7,054 bp linear DNA
DQ004563.1 GI: 63259393
545. Streptococcus mitis strain SK609 IgA1 protease (iga) gene, complete cds 7,553 bp linear DNA
DQ004562.1 GI: 63259391
546. Serratia proteamaculans ProA and Inh genes, complete cds 5,644 bp linear DNA AY818193.1
GI: 60461913
547. Streptococcus mutans strain GS-5 collagenase gene, complete cds 1,287 bp linear DNA AY644675.1
GI: 49473536
548. Photorhabdus sp. Az29 SAM-dependent methyltransferase-like protein, secreted alkaline
metalloprotease (prtA), putative PrtA-specific inhibitor precursor (inh), PrtB (prtB), PrtC (prtC), and PrtD
(prtD) genes, complete cds 9,270 bp linear DNA AY531111.1 GI: 42541020
549. Flavobacterium columnare strain G4 membrane-associated zinc metalloprotease gene, complete cds
1,800 bp linear DNA AY387596.1 GI: 37694418
550. Diachasmimorpha longicaudata entomopoxvirus putative metalloprotease gene, complete cds 2,063 bp
linear DNA AY598433.1 GI: 51317194
551. Myxococcus xanthus isolate C257/780 hypothetical adventurous gliding motility protein M (agmM),
hypothetical TPR-like protein, and adventurous gliding motility protein R (agmR) genes, complete cds 4,461
bp linear DNA AY197568.1 GI: 29169120
552. Caulobacter crescentus S-layer editing metalloprotease SapA (sapA) gene, complete cds 2,330 bp linear
DNA AY064211.1 GI: 17978657
553. Listonella anguillarum strain TL-01 metalloprotease gene, complete cds 1,836 bp linear DNA
AY091854.1 GI: 20301820
554. Trypanosoma cruzi strain CL Brener clone chimeric cosmid Tccli14-1, 8,578 bp linear DNA
AC114396.1 GI: 19263256
555. Listonella anguillarum metalloprotease (emp) gene, complete cds 1,925 bp linear DNA AY046320.1
GI: 15705138
556. Burkholderia pseudomallei serine metalloprotease gene, complete cds 2,793 bp linear DNA AF254803.1
GI: 11177175
557. Bacillus pumilus strain col-J collagenase ColB precursor, gene, complete cds 1,269 bp linear DNA
GU139189.1 GI: 269316462
558. Bacillus pumilus strain col-J collagenase ColA precursor, gene, complete cds 930 bp linear DNA
GU139188.1 GI: 269316460
559. Sequence 26 from Patent WO2009125296 5,379 bp linear DNA HC054965.1 GI: 266634205
560. Listonella anguillarum empA gene for metalloprotease, isolate ayu-H080701 1,836 bp linear DNA
FM866242.2 GI: 264658033
561. Pseudomonas fluorescens strain CHA0 putative amino acid permease, putative D-aminopeptidase
(dmpA), metalloprotease (aprA), protease inhibitor (aprI), and ABC transporter protein (aprD) genes,
complete cds 6,745 bp linear DNA AY644718.1 GI: 50236478
562. Streptococcus pneumoniae zmpD gene for zinc metalloprotease D, complete cds, serotype: 14,
isolateion source: blood of Taiwanese children 5,325 bp linear DNA AB457847.1 GI: 199601653
563. Streptococcus pneumoniae zmpD gene for zinc metalloprotease D, complete cds, serotype: 14,
isolation source: pleural fluid of Taiwanese children 5,403 bp linear DNA AB457846.1 GI: 199601651
564. Streptococcus pneumoniae zmpD gene for zinc metalloprotease D, complete cds, serotype: 23A,
isolation source: blood of Taiwanese children 5,325 bp linear DNA AB457845.1 GI: 199601649
565. Sequence 7 from U.S. Pat. No. 7,572,5991,125 bp linear DNA GP512231.1 GI: 259278965
566. Sequence 5 from U.S. Pat. No. 7,572,599 1,128 bp linear DNA GP512230.1 GI: 259278964
567. Sequence 3 from U.S. Pat. No. 7,572,599 1,128 bp linear DNA GP512229.1 GI: 259278963
568. Sequence 1 from U.S. Pat. No. 7,572,599 2,743 bp linear DNA GP512228.1 GI: 259278962
569. Sequence 61 from U.S. Pat. No. 7,557,079 1,313 bp linear DNA GP468192.1 GI: 259219215
570. Sequence 58 from U.S. Pat. No. 7,557,079 1,048 bp linear DNA GP468191.1 GI: 259219214
571. Sequence 57 from U.S. Pat. No. 7,557,079 1,554 bp linear DNA GP468190.1 GI: 259219213
572. Sequence 56 from U.S. Pat. No. 7,557,079 1,103 bp linear DNA GP468189.1 GI: 259219212
573. Sequence 39 from U.S. Pat. No. 7,557,079 5,269 bp linear DNA GP468176.1 GI: 259219199
574. Sequence 38 from U.S. Pat. No. 7,557,079 5,005 bp linear DNA GP468175.1 GI: 259219198
575. Sequence 35 from U.S. Pat. No. 7,557,079 1,224 bp linear DNA GP468174.1 GI: 259219197
576. Sequence 33 from U.S. Pat. No. 7,557,079 1,293 bp linear DNA GP468173.1 GI: 259219196
577. Sequence 31 from U.S. Pat. No. 7,557,079 419 bp linear DNA GP468172.1 GI: 259219195
578. Sequence 13 from U.S. Pat. No. 7,557,079 1,008 bp linear DNA GP468163.1 GI: 259219186
579. Sequence 11 from U.S. Pat. No. 7,557,079 419 bp linear DNA GP468162.1 GI: 259219185
580. DIAGNOSTIC METHOD FOR OVARIAN CANCER 3,546 bp linear DNA DM169318.1
GI: 256625211
581. Paenibacillus polymyxa npr gene for extracellular neutral protease, complete cds 2,418 bp linear DNA
D00861.1 GI: 216307
582. Pseudoalteromonas sp. SM495 metalloprotease E495 gene, complete cds 2,193 bp linear DNA
FJ211191.1 GI: 208436008
583. Pseudoalteromonas sp. SM9913 secreted metalloprotease Mcp02 (mcp02) gene, complete cds 2,184 bp
linear DNA EF029091.2 GI: 123959779
584. Vibrio tubiashii strain ATCC 19105 VtpA (vtpA) gene, complete cds 1,824 bp linear DNA FJ455121.1
GI: 217040065
585. Vibrio tubiashii strain 00-90-6 VtpA (vtpA) gene, complete cds 1,824 bp linear DNA FJ455120.1
GI: 217040063
586. Vibrio tubiashii strain X00-12-1 VtpA gene, complete cds 1,824 bp linear DNA FJ455119.1
GI: 217040061
587. Streptomyces cacaoi extracellular metalloprotease (npr) gene 1,837 bp linear DNA M37055.1
GI: 153374
588. Myroides profundi strain D25 myroilysin precursor, gene, complete cds 1,947 bp linear DNA
EU883966.2 GI: 197944805
589. Streptomyces parvulus subsp. citrinus gene for collagenase precursor, complete cds 2,894 bp linear
DNA AB429498.1 GI: 206725170
590. Vibrio mimicus vmp gene for metalloprotease, complete cds, strain: ES-39 1,836 bp linear DNA
AB435238.1 GI: 186704297
591. Staphylococcus pseudintermedius pst gene for metalloproteinase, strain CCUG 49543 1,560 bp linear
DNA AM921785.1 GI: 218090026
592. Staphylococcus pseudintermedius pst gene for metalloproteinase, strain 9p 1,518 bp linear DNA
AM921784.1 GI: 218090024
593. Staphylococcus pseudintermedius pst gene for metalloproteinase, strain 6p 1,518 bp linear DNA
AM921783.1 GI: 218090021
594. Staphylococcus pseudintermedius pst gene for metalloproteinase, strain 6INT 1,518 bp linear DNA
AM921782.1 GI: 218090018
595. Staphylococcus pseudintermedius pst gene for metalloproteinase, strain 2INT 1,518 bp linear DNA
AM921781.1 GI: 218090016
596. Chlamydomonas reinhardtii mmp2 gene, 3′UTR, strain: CC-1373 417 bp linear DNA AB071886.1
GI: 19570936
597. Chlamydomonas reinhardtii mmp2 gene, 3′UTR, strain: 6145c 417 bp linear DNA AB071885.1
GI: 19570935
598. Chlamydomonas reinhardtii mmp2 gene, 3′UTR, strain: 21 gr 431 bp linear DNA AB071884.1
GI: 19570934
599. Chlamydomonas reinhardtii mmp1 gene for gametolysin, 3′UTR, strain: CC-2938 403 bp linear DNA
AB071883.1 GI: 19570933
600. Chlamydomonas reinhardtii mmp1 gene for gametolysin, 3′UTR, strain: CC-2937 375 bp linear DNA
AB071882.1 GI: 19570932
601. Chlamydomonas reinhardtii mmp1 gene for gametolysin, 3′UTR, strain: CC-2936 359 bp linear DNA
AB071881.1 GI: 19570931
602. Chlamydomonas reinhardtii mmp1 gene for gametolysin, 3′UTR, strain: CC-2935 75 bp linear DNA
AB071880.1 GI: 19570930
603. Chlamydomonas reinhardtii mmp1 gene for gametolysin, 3′UTR, strain: CC-2932 358 bp linear DNA
AB071879.1 GI: 19570929
604. Chlamydomonas reinhardtii mmp1 gene for gametolysin, 3′UTR, strain: CC-2931 358 bp linear DNA
AB071878.1 GI: 19570928
605. Chlamydomonas reinhardtii mmp1 gene for gametolysin, 3′UTR, strain: CC-2344 358 bp linear DNA
AB071877.1 GI: 19570927
606. Chlamydomonas reinhardtii mmp1 gene for gametolysin, 3′UTR, strain: CC-2342 358 bp linear DNA
AB071876.1 GI: 19570926
607. Chlamydomonas reinhardtii mmp1 gene for gametolysin, 3′UTR, strain: CC-1952 404 bp linear DNA
AB071875.1 GI: 19570925
608. Chlamydomonas reinhardtii mmp1 gene for gametolysin, 3′UTR, strain: CC-1373 344 bp linear DNA
AB071874.1 GI: 19570924
609. Chlamydomonas reinhardtii fus1 gene, intron, strain: 21 gr 525 bp linear DNA AB071888.1
GI: 19570938
610. Chlamydomonas reinhardtii mmp2 gene for matrix metalloprotease 2, complete cds 5,628 bp linear
DNA AB058412.1 GI: 15718392
611. Chlamydomonas reinhardtii mmp1 gene for gametolysin, complete cds 6,100 bp linear DNA
AB058411.1 GI: 15718390
612. Chlamydomonas smithii fus1 gene, intron 12, strain: CC-1373 530 bp linear DNA AB071889.1
GI: 19570939
613. Trichophyton equinum metalloprotease 4 gene, complete cds 2,318 bp linear DNA FJ356719.1
GI: 209976059
614. cDNA arrays and their use for gene expression profiling 782 bp linear DNA DL128666.1 GI: 207066494
615. cDNA arrays and their use for gene expression profiling 466 bp linear DNA DL128665.1 GI: 207066493
616. cDNA arrays and their use for gene expression profiling 437 bp linear DNA DL128664.1 GI: 207066492
617. Salinivibrio proteolyticus zinc metalloprotease precursor (svp) gene, complete cds 1,836 bp linear DNA
DQ908958.1 GI: 115291369
618. Serratia sp. KCK metalloprotease precursor, gene, complete cds 1,936 bp linear DNA EF191201.1
GI: 124518423
619. Biomarkers for Monitoring IMPDH Pathway Inhibition 1,973 bp linear DNA DL081259.1
GI: 197266245
620. Sequence 5 from U.S. Pat. No. 7,399,832 2,604 bp linear DNA GC503176.1 GI: 197032732
621. Sequence 2 from U.S. Pat. No. 7,399,832 2,499 bp linear DNA GC503175.1 GI: 197032731
622. Vibrio tubiashii strain RE22 VtpA (vtpA) gene, complete cds 1,824 bp linear DNA EU675309.1
GI: 189309493
623. Vibrio tubiashii strain RE22 VthB (vthB) and VthA (vthA) genes, complete cds 1,924 bp linear DNA
EU675308.1 GI: 189309490
624. Sequence 3 from U.S. Pat. No. 7,374,933 6,306 bp linear DNA EA758131.1 GI: 189862148
625. Sequence 1 from U.S. Pat. No. 7,374,933 2,115 bp linear DNA EA758130.1 GI: 189862147
626. Homo sapiens DNA, upstream region of type I collagenase 721 bp linear DNA D26110.1 GI: 439714
627. Novel Human Metalloprotease and Polynucleotides Encoding the Same 6,306 bp linear DNA
DJ387844.1 GI: 188563217
628. Novel Human Metalloprotease and Polynucleotides Encoding the Same 2,115 bp linear DNA
DJ387843.1 GI: 188563216
629. Candidatus Liberibacter asiaticus zinc metalloprotease (zmpA) gene, complete cds 1,147 bp linear DNA
EF164804.1 GI: 140063938
630. Vibrio vulnificus vvp gene for metalloprotease, type strain CECT 4602T 3,045 bp linear DNA
AM492792.1 GI: 169642808
631. Streptomyces lividans Imp gene for metalloprotease precursor, complete cds 1,319 bp linear DNA
D00670.1 GI: 217015
632. Inhibitor of platelets activation and aggregation comprisingheompexin domain in matrix metalloprotease
582 bp linear DNA DI166877.1 GI: 168455993
633. Inhibitor of platelets activation and aggregation comprisingheompexin domain in matrix metalloprotease
576 bp linear DNA DI166204.1 GI: 168455535
634. Serratia sp. A2 grimelysin precursor, gene, complete cds 1,026 bp linear DNA EU287453.1
GI: 163716943
635. Serratia grimesii strain DSMZ 30063 grimelysin precursor, gene, complete cds 1,026 bp linear DNA
EU287452.1 GI: 163716941
636. Clostridium perfringens virR gene for positive regulator for virulence factors, complete cds 1,929 bp
linear DNA D14877.1 GI: 473705
637. Clostridium perfringens colA gene for collagenase, complete cds 4,141 bp linear DNA D13791.1
GI: 440850
638. Mus musculus gene for 92-kDa type IV collagenase, promoter region 602 bp linear DNA D15060.1
GI: 286066
639. Sequence 5 from U.S. Pat. No. 7,312,321 2,262 bp linear DNA EA371961.1 GI: 167243848
640. Sequence 3 from U.S. Pat. No. 7,312,321 2,340 bp linear DNA EA371960.1 GI: 167243847
641. Sequence 1 from U.S. Pat. No. 7,312,321 2,076 bp linear DNA EA371959.1 GI: 167243846
642. Burkholderia pseudomallei isolate D286 microbial collagenase gene, complete cds 1,944 bp linear DNA
EU312003.1 GI: 163639394
643. Photorhabdus sp. Az29 PrtS (prtS) gene, complete cds 1,953 bp linear DNA EU307118.1 GI: 162957284
644. Homo sapiens gene for 92-kDa type IV collagenase, 5′-flanking region 2,226 bp linear DNA D10051.1
GI: 219891
645. Sequence 5229 from Patent WO2007106407 708 bp linear DNA CS818320.1 GI: 162792129
646. Sequence 3618 from Patent WO2007106407 4,452 bp linear DNA CS816709.1 GI: 162771844
647. Sequence 7341 from Patent WO2007106407 4,131 bp linear DNA CS820432.1 GI: 162771176
648. Sequence 29 from U.S. Pat. No. 7,285,633 733 bp linear DNA EA304909.1 GI: 162444094
649. Sequence 1 from U.S. Pat. No. 7,285,633 1,707 bp linear DNA EA304904.1 GI: 162444089
650. Streptomyces nigrescens smpi gene for metalloprotease inhibitor, complete cds 1,142 bp linear DNA
D00671.1 GI: 217027
651. Pseudomonas fluorescens prrB gene for putative regulatory RNA, strain M114 564 bp linear DNA
AJ556798.1 GI: 30014091
652. Clostridium histolyticum orfluG, colG, mscL, orf2dG, orf3dG genes, complete cds 5,914 bp linear DNA
D87215.1 GI: 4760823
653. Aeromonas sobria strain 288 metalloprotease gene, complete cds 3,319 bp linear DNA DQ784565.1
GI: 110748601
654. Vibrio parahaemolyticus metalloprotease gene, complete cds 2,445 bp linear DNA DQ479431.1
GI: 94449172
655. Serratia marcescens strain HR-3 insecticidal protein gene, complete cds 1,464 bp linear DNA
EF070725.1 GI: 117655426
656. Pseudomonas aeruginosa strain K organic solvent tolerant elastase gene, complete cds 2,000 bp linear
DNA EU021222.1 GI: 15412704
657. Lactobacillus helveticus strain WSU19 endopeptidase O3 (pepO3) gene, complete cds 3,407 bp linear
DNA DQ221766.1 GI: 78191628
658. Lactobacillus helveticus strain WSU19 endopeptidase O (pepO) gene, complete cds 4,334 bp linear
DNA DQ221765.1 GI: 78191626
659. Lactobacillus helveticus strain WSU19 endopeptidase O2 (pepO2) gene, complete cds 5,765 bp linear
DNA DQ221763.1 GI: 78191622
660. Sequence 12 from Patent WO2007044993 1,566 bp linear DNA CS608253.1 GI: 148917206
661. Sequence 2 from Patent WO2007044993 1,578 bp linear DNA CS608243.1 GI: 148917198
662. Sequence 1 from Patent WO2007044993 713 bp linear DNA CS608242.1 GI: 148917197
663. Flavobacterium columnare collagenase gene, complete cds 2,152 bp linear DNA EF501979.1
GI: 145652155
664. Clostridium botulinum botulinum neurotoxin type A (bonta) gene, complete cds 3,891 bp linear DNA
EF506572.1 GI: 145226691
665. Sequence 5 from U.S. Pat. No. 7,196,168 2,604 bp linear DNA EA105472.1 GI: 145041986
666. Sequence 2 from U.S. Pat. No. 7,196,168 2,499 bp linear DNA EA105471.1 GI: 145041985
667. Sequence 5 from U.S. Pat. No. 7,189,525 2,262 bp linear DNA EA087929.1 GI: 144995372
668. Sequence 3 from U.S. Pat. No. 7,189,525 2,340 bp linear DNA EA087928.1 GI: 144995371
669. Sequence 1 from U.S. Pat. No. 7,189,525 2,076 bp linear DNA EA087927.1 GI: 14499537
670. Uncultured bacterium clone ES63H9 metalloprotease gene, complete cds 2,427 bp linear DNA
EF100137.1 GI: 118499462
671. METALLOPROTEASE ACTIVATION OF MYOSTATIN, AND METHODS OF MODULATING
MYOSTATIN ACTIVITY 1,125 bp linear DNA DD408580.1 GI: 126151630
672. METALLOPROTEASE ACTIVATION OF MYOSTATIN, AND METHODS OF MODULATING
MYOSTATIN ACTIVITY 1,128 bp linear DNA DD408579.1 GI: 126151629
673. METALLOPROTEASE ACTIVATION OF MYOSTATIN, AND METHODS OF MODULATING
MYOSTATIN ACTIVITY 1,128 bp linear DNA DD408578.1 GI: 126151628
674. METALLOPROTEASE ACTIVATION OF MYOSTATIN, AND METHODS OF MODULATING
MYOSTATIN ACTIVITYn2,743 bp linear DNA DD408577.1 GI: 126151627
675. Metalloprotease Proteins 5,269 bp linear DNA DD406973.1 GI: 126150224
676. Metalloprotease Proteins 5,005 bp linear DNA DD406972.1 GI: 126150223
677. Metalloprotease Proteins 1,224 bp linear DNADD406971.1 GI: 126150222
678. Metalloprotease Proteins 1,293 bp linear DNA DD406970.1 GI: 126150221
679. Metalloprotease Proteins 419 bp linear DNA DD406969.1 GI: 126150220
680. Metalloprotease Proteins 1,008 bp linear DNA DD406960.1 GI: 126150211
681. Metalloprotease Proteins 419 bp linear DNADD406959.1 GI: 126150210
682. Listeria monocytogenes strain CCTCCAB97021 Mpl (mpl) gene, complete cds 1,569 bp linear DNA
EF183452.1 GI: 124495032
683. Streptococcus pneumoniae zmpB gene for putative zinc metalloprotease, complete cds, strain: NTUH-
p3 5,634 bp linear DNA AB292311.1 GI: 124430473
684. Streptococcus pneumoniae zmpB gene for putative zinc metalloprotease, complete cds, strain: NTUH-
p28 5,63 1 bp linear DNA AB292310.1 GI: 124430471
685. Streptococcus pneumoniae zmpB gene for putative zinc metalloprotease, complete cds 5,63 1 bp linear
DNA AB292309.1 GI: 124430469
686. A.fumigatus (delta18) gene for metalloprotease (MEP) 3,075 bp linear DNA Z30424.1 GI: 3776612
687. H.sapiens promoter region of collagenase 3 gene 1,202 bp linear DNA X81640.1 GI: 1945758
688. P.lividus Bp10 gene for blastula protease-10 6,003 bp linear DNA X65721.1 GI: 1419727
689. P.lividus gene for hatching enzyme 6,332 bp linear DNA X65722.1 GI: 416551
690. METALLOPROTEASE ACTIVATION OF MYOSTATIN, AND METHODS OF MODULATING
MYOSTATIN ACTIVITY 1,125 bp linear DNA DD349667.1 GI: 116558699
691. METALLOPROTEASE ACTIVATION OF MYOSTATIN, AND METHODS OF MODULATING
MYOSTATIN ACTIVITY 1,128 bp linear DNA DD349666.1 GI: 116558697
692. METALLOPROTEASE ACTIVATION OF MYOSTATIN, AND METHODS OF MODULATING
MYOSTATIN ACTIVITY 1,128 bp linear DNA DD349665.1 GI: 116558695
693. METALLOPROTEASE ACTIVATION OF MYOSTATIN, AND METHODS OF MODULATING
MYOSTATIN ACTIVITY 2,743 bp linear DNA DD349664.1 GI: 116558693
694. Bacillus vietnamensis gene for protease, complete cds 1,991 bp linear DNA AB174895.1 GI: 4573660
695. Erwinia chrysanthemi EprB (eprB) gene, complete cds 1,443 bp linear DNAAY919873.1 GI: 60101755
696. Erwinia chrysanthemi extracellular metalloprotease EprC (eprC) gene, complete cds 1,440 bp linear
DNA AF541945.1 GI: 23452666
697. Sequence 5 from U.S. Pat. No. 7,083,965 2,262 bp linear DNA AR914278.1 GI: 112103237
698. Sequence 3 from U.S. Pat. No. 7,083,965 2,340 bp linear DNA AR914277.1 GI: 112103236
699. Sequence 1 from U.S. Pat. No. 7,083,965 2,076 bp linear DNA AR914276.1 GI: 112103234
700. Sequence 14 from U.S. Pat. No. 7,049,412 3,250 bp linear DNA AR882052.1 GI: 111982530
701. Sequence 1 from U.S. Pat. No. 7,049,412 4,192 bp linear DNA AR882045.1 GI: 111982523
702. Sequence 29 from U.S. Pat. No. 7,034,134 733 bp linear DNA AR862074.1 GI: 111950411
703. Sequence 1 from U.S. Pat. No. 7,034,134 1,707 bp linear DNA AR862069.1 GI: 111950406
704. Uncultured microorganism r15 gene for laccase and ORF1, ORF3, ORF4 and ORF5 DNA for
hypothetical proteins, isolated from bovine rumen 5,594 bp linear DNA AM269758.1 GI: 108248032
705. Assays for inhibitors of FtsH 1,911 bp linear DNA BD261530.1 GI: 33071298
706. Assays for inhibitors of FtsH 1,932 bp linear DNA BD261529.1 GI: 33071297
707. Novel membrane metalloprotease NEPII and therapeutically useful utilization thereof for screening
inhibitory factor 327 bp linear DNA BD204630.1 GI: 33014400
708. Novel membrane metalloprotease NEPII and therapeutically useful utilization thereof for screening
inhibitory factor 2,765 bp linear DNA BD204629.1 GI: 33014399
709. Novel metalloprotease 1,856 bp linear DNA BD196411.1 GI: 33006181
710. Novel metalloprotease 2,426 bp linear DNA BD196410.1 GI: 33006180
711. Sequence 39 from Patent WO2004056983 5,269 bp linear DNA CS226118.1 GI: 83690528
712. Sequence 38 from Patent WO2004056983 5,005 bp linear DNA CS226117.1 GI: 83690527
713. Sequence 35 from Patent WO2004056983 1,224 bp linear DNA CS226114.1 GI: 83690526
714. Sequence 33 from Patent WO200405698 1,293 bp linear DNA CS226112.1 GI: 8369052
715. Sequence 31 from Patent WO2004056983 419 bp linear DNA CS226110.1 GI: 8369052
716. Sequence 13 from Patent WO2004056983 1,008 bp linear DNA CS226092.1 GI: 83690515
717. Sequence 11 from Patent WO2004056983 419 bp linear DNA CS226090.1 GI: 83690514
718. Methods of Diagnosing & Treating Diabetes and Insulin Resistance 3,865 bp linear DNA DD261317.1
GI: 109253626
719. Candida albicans gene for aspartyl proteinase (EC 3.4.23.6) 1,736 bp linear DNA X13669.1 GI: 2508
720. Bacillus thuringiensis metalloprotease enhancin (mpbe) gene, promoter region and complete cds 2,464
bp linear DNA DQ151839.1 GI: 73913014
721. METHODS FOR USING ADAMTS 12, AN INTEGRIN AND METALLOPROTEASE WITH
THROMBOSPONDIN MOTIFS 5,070 bp linear DNA DD246859.1 GI: 94033734
722. Novel membrane-bound metalloprotease 2,118 bp linear DNA E39430.1 GI: 18621539
723. Novel membrane-bound metalloprotease 1,938 bp linear DNA E39423.1 GI: 18621532
724. Human disintegrin metalloprotease relating to Drosophila KUZ gene 3,349 bp linear DNA E33199.1
GI: 13026944
725. Modified matrix metalloprotease 3 537 bp linear DNA E27504.1 GI: 13026499
726. Modified matrix metalloprotease 3 537 bp linear DNA E27503.1 GI: 13026498
727. Modified matrix metalloprotease 3 537 bp linear DNA E27502.1 GI: 13026497
728. Modified matrix metalloprotease 3 537 bp linear DNA E27501.1 GI: 13026496
729. Modified matrix metalloprotease 3 1,434 bp linear DNA E27500.1 GI: 13026495
730. cDNA encoding a protein which has matrixmetalloproteinase activity, SMCP-1 2,052 bp linear DNA
E17223.1 GI: 5711906
731. cDNA encoding novel rat liver metalloprotease 1,551 bp linear DNA E15631.1 GI: 5710314
732. cDNA encoding novel human liver metalloprotease 1,524 bp linear DNA E15630.1 GI: 5710313
733. cDNA encoding membrane type matrix metalloprotease 3,MT-MMP-3 2,107 bp linear DNA E12862.1
GI: 3251694
734. DNA encoding collagenase 4,054 bp linear DNA E03106.1 GI: 2171323
735. Human Disintegrin Metalloprotease Related to Drosophila KUZ gene 3,349 bp linear DNA BD412822.1
GI: 92289150
736. A protease, a gene therefor and the use thereof 2,481 bp linear DNA BD355647.1 GI: 92256767
737. Novel human enzyme of the metalloprotease family 2,262 bp linear DNA BD313651.1 GI: 92161403
738. Novel human enzyme of the metalloprotease family 2,340 bp linear DNA BD313650.1 GI: 92161402
739. Novel human enzyme of the metalloprotease family 2,076 bp linear DNA BD313649.1 GI: 92161401
740. Novel metalloprotease having an activity of aggrecanase 3,455 bp linear DNA BD301438.1
GI: 92150143
741. Novel metalloprotease having an activity of aggrecanase 3,462 bp linear DNA BD301437.1
GI: 92150142
742. Novel metalloprotease having an activity of aggrecanase 3,467 bp linear DNA BD301436.1
GI: 92150141
743. Novel metalloprotease having an activity of aggrecanase 3,470 bp linear DNA BD301435.1
GI: 92150140
744. Novel metalloprotease having an activity of aggrecanase 3,469 bp linear DNA BD301434.1
GI: 92150139
745. Novel metalloprotease having an activity of aggrecanase 3,464 bp linear DNA BD301433.1
GI: 92150138
746. Novel metalloprotease having an activity of aggrecanase 3,467 bp linear DNA BD301432.1
GI: 92150137
747. Novel metalloprotease having an activity of aggrecanase 3,473 bp linear DNA BD301431.1
GI: 92150136
748. Novel metalloprotease having an activity of aggrecanase 2,853 bp linear DNA BD301409.1
GI: 92150114
749. Chlamydia trachomatis serovar L2 mutant polypeptide deformylase gene, complete cds 846 bp linear
DNA DQ287335.1 GI: 83033879
750. Chlamydia trachomatis serovar L2 polypeptide deformylase gene, complete cds 846 bp linear DNA
DQ287334.1 GI: 83033877
751. Uncultured prokaryote HSP gene 1,837 bp linear DNA Y09872.2 GI: 89145246
752. Sus scrofa ADAMTS1 (ADAMTS1) gene, complete cds 9,026 bp linear DNA DQ177331.1
GI: 76781336
753. Aeromonas punctata ap19 gene for metalloprotease, complete cds 2,465 bp linear DNA AB195996.1
GI: 84579468
754. ADAMTS9_v477 Vervet DNA PCR Cercopithecus aethiops STS genomic clone VMA1430, sequence
tagged site 841 bp linear DNA BV680058.1 GI: 83627382
755. Unidentified microorganism phagemid clone pBKR.41 2,802 bp linear DNA AM050332.1 GI: 82524086
756. Aeromonas hydrophila strain AH-1 extracellular protease (mepA) gene, complete cds 1,044 bp linear
DNA AY841796.1 GI: 61658216
757. Vibrio alginolyticus strain ZJ04107 collagenase gene, complete cds 2,449 bp linear DNA DQ097161.1
GI: 70672276
758. Sequence 623 from Patent WO2005054508 2,722 bp linear DNA CS118576.1 GI: 70666522
759. Sequence 622 from Patent WO2005054508 290 bp linear DNA CS118575.1 GI: 70666521
760. E.chrysanthemi gene for protease A 2,368 bp linear DNA X70011.1 GI: 297897
761. E.chrysanthemi gene for protease G 3,200 bp linear DNA X71365.1 GI: 297860
762. S.coelicolor genes for metalloproteinase and LysR-type transcriptional activator 4,673 bp linear DNA
Z11929.1 GI: 46866
763. Sequence 5 from U.S. Pat. No. 6,855,532 2,262 bp linear DNA AR636932.1 GI: 62770139
764. Sequence 3 from U.S. Pat. No. 6,855,532 2,340 bp linear DNA AR636931.1 GI: 62770138
765. Sequence 1 from U.S. Pat. No. 6,855,532 2,076 bp linear DNA AR636930.1 GI: 62770137
766. Renibacterium salmoninarum hly gene for metalloproteas 1,870 bp linear DNA X76499.2 GI: 18250640
767. Erwinia amylovora prt operon 6,652 bp linear DNA Y19002.1 GI: 4826414
768. C.perfringens NCTC8237 pbg, arcA, arcB genes 2,645 bp linear DNA X97684.1 GI: 1321792
769. C.perfringens strain 13 arcABDC, ahrC and colA genes 5,447 bp linear DNA X97768.1 GI: 1321785
770. L.longbeachae mspA gene 2,754 bp linear DNA X83035.1 GI: 758139
771. S.epidermis gene for protease 1,853 bp linear DNA X69957.1 GI: 396258
772. L. monocytogenes mpl gene for metalloprotease 1,952 bp linear DNA X54619.1 GI: 44114
773. B.brevis gene for neutral protease 2,355 bp linear DNA X61286.1 GI: 39378
774. Uncultured bacterium gene for putative clp protease, clone 11 2,850 bp linear DNA AJ488200.1
GI: 57282290
775. Uncultured bacterium gene for putative zinc metallopeptidase, clone 8 1,884 bp linear DNA AJ488199.1
GI: 57282288
776. Uncultured bacterium gene for putative heat shock protein 20, clone 4 570 bp linear DNA AJ488197.1
GI: 57282286
777. Uncultured bacterium gene for putative thioredoxin, clone 2 501 bp linear DNA AJ488196.1
GI: 57282284
778. Yersinia ruckeri inh gene, pD gene, pE gene and pF gene, strain 150 5,757 bp linear DNA AJ421517.1
GI: 17426961
779. Yersinia ruckeri p1 gene for metalloprotease p1 2,082 bp linear DNA AJ318052.1 GI: 14161133
780. Bradyrhizobium japonicum yaeN and ftsH genes 3,715 bp linear DNA AJ243808.1 GI: 5531225
781. Pseudomonas tolaasii eprA, eprI, eprD, eprE and eprF genes 6,900 bp linear DNA AJ007827.1
GI: 3646410
782. Bacillus subtilis strain KCTC 3014 PepT gene, complete cds 1,233 bp linear DNA AY960131.1
GI: 62002103
783. Sequence 1 from U.S. Pat. No. 6,825,025 3,377 bp linear DNA AR609741.1 GI: 56665141
784. Sequence 1 from U.S. Pat. No. 6,825,022 2,968 bp linear DNA AR609700.1 GI: 56665046
785. Sequence 5 from U.S. Pat. No. 6,787,644 2,604 bp linear DNA AR580555.1 GI: 56610967
786. Sequence 2 from U.S. Pat. No. 6,787,644 2,499 bp linear DNA AR580554.1 GI: 56610966
787. ADAMTS6 3045 Rhesus macaque genomic DNA Macaca mulatta STS genomic clone MMA3045,
sequence tagged site 577 bp linear DNA BV209051.1 GI: 51853595
788. ADAMTS19_2956 Rhesus macaque genomic DNA Macaca mulatta STS genomic clone MMA2956,
sequence tagged site 505 bp linear DNA BV209050.1 GI: 51853594
789. ADAMTS13 3006 Rhesus macaque genomic DNA Macaca mulatta STS genomic clone MMA3006,
sequence tagged site 559 bp linear DNA BV209049.1 GI: 51853593
790. ADAMTS8_2450 Rhesus macaque genomic DNA Macaca mulatta STS genomic clone MMA2450,
sequence tagged site 747 bp linear DNA BV208363.1 GI: 49533046
791. ADAMTS14_2587 Rhesus macaque genomic DNA Macaca mulatta STS genomic clone MMA2587,
sequence tagged site654 bp linear DNA BV208362.1 GI: 49533045
792. ADAMTS9_1430 Rhesus macaque genomic DNA Macaca mulatta STS genomic clone MMA1430,
sequence tagged site 840 bp linear DNA BV165974.1 GI: 47776355
793. ADAMTS3_678 Rhesus macaque genomic DNA Macaca mulatta STS genomic clone MMA678,
sequence tagged site 814 bp linear DNA BV165973.1 GI: 47776354
794. ADAMTS1_209 Rhesus macaque genomic DNA Macaca mulatta STS genomic clone MMA209,
sequence tagged site 797 bp linear DNA BV165972.1 GI: 47776353
795. ADAMTS20 4252 Rhesus macaque genomic DNA Macaca mulatta STS genomic clone MMA4252,
sequence tagged site 482 bp linear DNA BV210459.1 GI: 55167472
796. Uncultured bacterium gene for putative transcription factor, clone 7 1,179 bp linear DNA AJ488198.1
GI: 55057253
797. Sequence 2 from Patent EP1433845 3,079 bp linear DNA CQ827891.1 GI: 4973145
798. MARC3917-3918 Bovine white blood cells Bos taurus STS genomic, sequence tagged site 792 bp linear
DNA G67671.1 GI: 12802959
799. Sequence 31 from U.S. Pat. No. 6,716,613 3,455 bp linear DNA AR492145.1 GI: 47260648
800. Sequence 30 from U.S. Pat. No. 6,716,613 3,462 bp linear DNA AR492144.1 GI: 47260647
801. Sequence 29 from U.S. Pat. No. 6,716,613 3,467 bp linear DNA AR492143.1 GI: 47260646
802. Sequence 28 from U.S. Pat. No. 6,716,613 3,470 bp linear DNA AR492142.1 GI: 47260645
803. Sequence 27 from U.S. Pat. No. 6,716,613 3,469 bp linear DNA AR492141.1 GI: 47260644
804. Sequence 26 from U.S. Pat. No. 6,716,613 3,464 bp linear DNA AR492140.1 GI: 47260643
805. Sequence 25 from U.S. Pat. No. 6,716,613 3,467 bp linear DNA AR492139.1 GI: 47260642
806. Sequence 24 from U.S. Pat. No. 6,716,613 3,473 bp linear DNA AR492138.1 GI: 47260641
807. Sequence 2 from U.S. Pat. No. 6,716,613 2,853 bp linear DNA AR492116.1 GI: 47260619
808. Vibrio vulnificus vvp gene for protease, complete cds 1,830 bp linear DNA AB084580.1 GI: 24636579
809. Sequence 1 from U.S. Pat. No. 6,664,093 3,377 bp linear DNA AR438769.1 GI: 42663758
810. Sequence 27 from U.S. Pat. No. 6,642,041 733 bp linear DNA AR428810.1 GI: 40188596
811. Sequence 23 from U.S. Pat. No. 6,642,041 1,526 bp linear DNA AR428809.1 GI: 40188595
812. Sequence 21 from U.S. Pat. No. 6,642,041 1,387 bp linear DNA AR428808.1 GI: 40188594
813. Sequence 20 from U.S. Pat. No. 6,642,041 14,364 bp linear DNA AR428807.1 GI: 40188593
814. Sequence 1 from U.S. Pat. No. 6,642,041 2,197 bp linear DNA AR428803.1 GI: 40188589
815. Sequence 163 from Patent WO03054178 891 bp linear DNA AX799416.1 GI: 37605269
816. Sequence 153 from Patent WO03054178 304 bp linear DNA AX799406.1 GI: 37605264
817. Sequence 151 from Patent WO03054178 1,494 bp linear DNA AX799404.1 GI: 37605263
818. Sequence 149 from Patent WO03054178 546 bp linear DNA AX799402.1 GI: 37605262
819. Sequence 139 from Patent WO03054178 304 bp linear DNA AX799392.1 GI: 37605257
820. Sequence 137 from Patent WO03054178 1,053 bp linear DNA AX799390.1 GI: 37605256
821. Sequence 135 from Patent WO03054178 411 bp linear DNA AX799388.1 GI: 37605255
822. Sequence 133 from Patent WO03054178 4,094 bp linear DNA AX799386.1 GI: 37605254
823. Sequence 129 from Patent WO03054178 2,172 bp linear DNA AX799384.1 GI: 37605253
824. Sequence 127 from Patent WO03054178 1,710 bp linear DNA AX799382.1 GI: 37605252
825. Sequence 99 from Patent WO03054178 291 bp linear DNA AX799354.1 GI: 37605238
826. Sequence 89 from Patent WO03054178 211 bp linear DNA AX799344.1 GI: 37605233
827. Sequence 57 from Patent WO03054178 254 bp linear DNA AX799312.1 GI: 37605217
828. Sequence 53 from Patent WO03054178 362 bp linear DNA AX799308.1 GI: 37605215
829. Sequence 41 from Patent WO03054178 302 bp linear DNA AX799306.1 GI: 37605214
830. Sequence 19 from Patent WO03054178 238 bp linear DNA AX799284.1 GI: 37605203
831. Sequence 17 from Patent WO03054178 233 bp linear DNA AX799282.1 GI: 37605202
832. Sequence 13 from Patent WO03054178 300 bp linear DNA AX799278.1 GI: 37605200
833. Sequence 9 from Patent WO03054178 258 bp linear DNA AX799274.1 GI: 37605198
834. Sequence 3 from Patent WO03054178 535 bp linear DNA AX799268.1 GI: 37605195
835. Sequence 23 from U.S. Pat. No. 6,566,116 2,049 bp linear DNA AR322595.1 GI: 33708395
836. Sequence 22 from U.S. Pat. No. 6,566,116 2,264 bp linear DNA AR322594.1 GI: 33708394
837. Sequence 12 from U.S. Pat. No. 6,566,116 1,272 bp linear DNA AR322586.1 GI: 33708386
838. Sequence 11 from U.S. Pat. No. 6,566,116 1,257 bp linear DNA AR322585.1 GI: 33708385
839. Sequence 10 from U.S. Pat. No. 6,566,116 1,248 bp linear DNA AR322584.1 GI: 33708384
840. Sequence 9 from U.S. Pat. No. 6,566,116 1,233 bp linear DNA AR322583.1 GI: 33708383
841. Sequence 8 from U.S. Pat. No. 6,566,116 1,551 bp linear DNA AR322582.1 GI: 33708382
842. Sequence 7 from U.S. Pat. No. 6,566,116 1,524 bp linear DNA AR322581.1 GI: 33708381
843. Sequence 4 from U.S. Pat. No. 6,548,284 2,823 bp linear DNA AR306358.1 GI: 31696134
844. Sequence 3 from U.S. Pat. No. 6,548,284 2,892 bp linear DNA AR306357.1 GI: 31696133
845. Psychrobacter sp. 16S rRNA gene, strain 116 1,426 bp linear DNA AJ272303.1 GI: 12054947
846. Vibrio fluvialis vfpA gene for metalloprotease, complete cds 2,376 bp linear DNA AB071709.1
GI: 19570827
847. Sequence 225 from Patent WO0246467 782 bp linear DNA AX587755.1 GI: 28212397
848. Clostridium histolyticum colH gene for collagenase, complete cds 3,500 bp linear DNA D29981.1
GI: 563954
849. Sequence 224 from Patent WO0246467 466 bp linear DNA AX587754.1 GI: 27656488
850. Sequence 223 from Patent WO0246467 437 bp linear DNA AX587753.1 GI: 27656487
851. Chlamydomonas reinhardtii mmp1 gene for gametolysin, 3′UTR, strain: 6145c 344 bp linear DNA
AB071873.1 GI: 19570923
852. Sequence 1 from U.S. Pat. No. 6,475,764 4,358 bp linear DNA AR243224.1 GI: 27290343
853. Sequence 1 from Patent WO02077241 2,968 bp linear DNA AX565631.1 GI: 26000967
854. Sequence 3 from Patent WO0226948 1,521 bp linear DNA AX527746.1 GI: 25172276
855. Sequence 1 from Patent WO0226948 2,784 bp linear DNA AX527744.1 GI: 25172275
856. Human serum amyloid A (GSAA1) gene, complete cds 6,943 bp linear DNA X13895.1 GI: 36305
857. DNAs and polypeptides of metalloprotease disintegrins SVPH3-13 and SVPH3-17 2,604 bp linear DNA
BD130424.1 GI: 23225369
858. DNAs and polypeptides of metalloprotease disintegrins SVPH3-13 and SVPH3-17 2,499 bp linear DNA
BD130423.1 GI: 23225368
859. Mammalian matrix metalloprotease 3,691 bp linear DNA BD128395.1 GI: 23223340
860. Mammalian matrix metalloprotease 3,695 bp linear DNA BD128394.1 GI: 23223339
861. Novel metalloprotease having thrombospondin domain and nucleic acid composition encoding the same
4,086 bp linear DNA BD079107.1 GI: 22624710
862. Novel metalloprotease having thrombospondin domain and nucleic acid composition encoding the same
5,338 bp linear DNA BD079106.1 GI: 22624709
863. Novel metalloprotease having thrombospondin domain and nucleic acid composition encoding the same
3,263 bp linear DNA BD079105.1 GI: 22624708
864. Novel metalloprotease having thrombospondin domain and nucleic acid composition encoding the same
2,879 bp linear DNA BD079104.1 GI: 22624707
865. Novel metalloprotease having thrombospondin domain and nucleic acid composition encoding the same
4,086 bp linear DNA BD079095.1 GI: 22624698
866. Novel metalloprotease having thrombospondin domain and nucleic acid composition encoding the same
5,338 bp linear DNA BD079094.1 GI: 22624697
867. Novel metalloprotease having thrombospondin domain and nucleic acid composition encoding the same
3,263 bp linear DNA BD079093.1 GI: 22624696
868. Novel metalloprotease having thrombospondin domain and nucleic acid composition encoding the same
2,879 bp linear DNA BD079092.1 GI: 22624695
869. Aggrecan-decomposable metalloprotease 3,250 bp linear DNA BD073450.1 GI: 22619053
870. Aggrecan-decomposable metalloprotease 4,192 bp linear DNA BD073443.1 GI: 22619046
871. Membrane-bound metalloprotease and soluble secretion type thereof 2,823 bp linear DNA BD062350.1
GI: 22607955
872. Membrane-bound metalloprotease and soluble secretion type thereof 2,892 bp linear DNA BD062349.1
GI: 22607954
873. Use of a novel disintegrin metalloprotease, its mutants, fragments and the like 2,625 bp linear DNA
BD057533.1 GI: 22603139
874. Use of a novel disintegrin metalloprotease, its mutants, fragments and the like 736 bp linear DNA
BD057532.1 GI: 22603138
875. Use of a novel disintegrin metalloprotease, its mutants, fragments and the like 239 bp linear DNA
BD057531.1 GI: 22603137
876. Use of a novel disintegrin metalloprotease, its mutants, fragments and the like 239 bp linear DNA
BD057530.1 GI: 22603136
877. Use of a novel disintegrin metalloprotease, its mutants, fragments and the like 2,763 bp linear DNA
BD057529.1 GI: 22603135
878. Use of a novel disintegrin metalloprotease, its mutants, fragments and the like 1,824 bp linear DNA
BD057528.1 GI: 22603134
879. Sequence 4 from Patent WO02057461 3,179 bp linear DNA AX481383.1 GI: 22316297
880. Sequence 1 from Patent WO02057461 1,668 bp linear DNA AX481380.1 GI: 22316295
881. Chlamydomonas reinhardtii ypt4 gene, intron 7, strain: 21 gr 325 bp linear DNA AB071887.1
GI: 19570937
882. Chlamydomonas reinhardtii mmp1 gene for gametolysin, 3′UTR, strain: 21 gr 359 bp linear DNA
AB071872.1 GI: 19570922
883. Sequence 2 from U.S. Pat. No. 6,399,371 2,275 bp linear DNA AR211785.1 GI: 21515200
884. Sequence 1 from U.S. Pat. No. 6,399,371 2,275 bp linear DNA AR211784.1 GI: 21515198
885. Sequence 7 from U.S. Pat. No. 6,342,374 780 bp linear DNA AR183887.1 GI: 20227856
886. Sequence 6 from U.S. Pat. No. 6,342,374 432 bp linear DNA AR183886.1 GI: 20227855
887. Sequence 5 from U.S. Pat. No. 6,342,374 703 bp linear DNA AR183885.1 GI: 20227854
888. Novel metalloprotease and gene of the same 2,670 bp linear DNA E55282.1 GI: 18629795
889. Novel metalloprotease and gene of the same 3,312 bp linear DNA E55273.1 GI: 18629786
890. Novel metalloprotease and gene of the same 5,061 bp linear DNA E55265.1 GI: 18629778
891. Sequence 3 from Patent WO0188155 3,402 bp linear DNAAX338539.1 GI: 18128944
892. Sequence 1 from Patent WO0188155 3,403 bp linear DNAAX338537.1 GI: 18128943
893. Sequence 13 from Patent WO0188156 2,445 bp linear DNA AX327757.1 GI: 18098063
894. Sequence 11 from Patent WO0188156 3,471 bp linear DNA AX327755.1 GI: 18098061
895. Sequence 10 from Patent WO0188156 3,132 bp linear DNA AX327754.1 GI: 18098060
896. Sequence 8 from Patent WO0188156 3,329 bp linear DNA AX327752.1 GI: 18098058
897. Sequence 3 from Patent WO0188156 3,207 bp linear DNA AX327747.1 GI: 18098057
898. Sequence 1 from Patent WO0188156 3,403 bp linear DNA AX327745.1 GI: 1809805
899. Aeromonas caviae gene for protease, complete cds 3,499 bp linear DNA AB022174.1 GI: 7768559
900. Sequence 3 from Patent WO0179509 6,306 bp linear DNA AX286493.1 GI: 17048613
901. Sequence 1 from Patent WO0179509 2, 115 bp linear DNA AX286491.1 GI: 17048612
902. Sequence 4 from U.S. Pat. No. 6,280,993 3,356 bp linear DNA AR166375.1 GI: 16241652
903. Sequence 8 from U.S. Pat. No. 6,255,064 2,625 bp linear DNA AR160345.1 GI: 16224116
904. Sequence 7 from U.S. Pat. No. 6,255,064 736 bp linear DNA AR160344.1 GI: 16224114
905. Sequence 6 from U.S. Pat. No. 6,255,064 239 bp linear DNA AR160343.1 GI: 16224112
906. Sequence 5 from U.S. Pat. No. 6,255,064 239 bp linear DNA AR160342.1 GI: 16224110
907. Sequence 3 from U.S. Pat. No. 6,255,064 2,763 bp linear DNA AR160341.1 GI: 16224108
908. Sequence 1 from U.S. Pat. No. 6,255,064 1,824 bp linear DNA AR160340.1 GI: 16224105
909. Sequence 2 from Patent WO0170950 483 bp linear DNA AX255838.1 GI: 16074879
910. Sequence 1 from Patent WO0155428 1,563 bp linear DNA AX206683.1 GI: 15394591
911. Porphyromonas gingivalis prtC gene, patient GDR8 1,005 bp linear DNA Y15768.1 GI: 4138332
912. Porphyromonas gingivalis prtC gene, patient Eli88 1,005 bp linear DNA Y15767.1 GI: 4138330
913. Porphyromonas gingivalis prtC gene, patient Eli85 1,005 bp linear DNA Y15766.1 GI: 4138328
914. Porphyromonas gingivalis prtC gene, patient Eli55 1,005 bp linear DNA Y15765.1 GI: 4138326
915. Porphyromonas gingivalis prtC gene, patient Eli47 1,005 bp linear DNA Y15764.1 GI: 4138324
916. Porphyromonas gingivalis prtC gene, patient d165 1,005 bp linear DNA Y15763.1 GI: 4138322
917. Porphyromonas gingivalis prtC gene, patient 53977 1,629 bp linear DNA Y15762.1 GI: 4138320
918. Sequence 5 from Patent WO0136610 2,262 bp linear DNA AX146980.1 GI: 14346251
919. Sequence 3 from Patent WO0136610 2,340 bp linear DNA AX146978.1 GI: 14346249
920. Sequence 1 from Patent WO0136610 2,076 bp linear DNA AX146976.1 GI: 14346247
921. Sequence 812 from Patent WO0118547 498 bp linear DNA AX094688.1 GI: 13510906
922. Sequence 1 from Patent WO0044913 1,083 bp linear DNA AX033699.1 GI: 10280382
923. Sequence 3 from Patent WO9953077 327 bp linear DNA AX014703.1 GI: 10040977
924. Sequence 1 from Patent WO9953077 2,765 bp linear DNA AX014701.1 GI: 10040975
925. Sequence 4 from U.S. Pat. No. 5,968,774 747 bp linear DNA AR080393.1 GI: 10007128
926. Sequence 1 from U.S. Pat. No. 5,968,774 2,052 bp linear DNA AR080392.1 GI: 10007127
927. Cytophaga sp. DNA for collagenase, complete cds 5,653 bp linear DNA D50600.1 GI: 849045
928. Sequence 15 from Patent WO9907856 1,856 bp linear DNA A99214.1 GI: 6782165
929. Sequence 13 from Patent WO9907856 2,426 bp linear DNA A99212.1 GI: 6782163
930. Sequence 1 from Patent WO9822574 4,358 bp linear DNA A91933.1 GI: 6740795
931. Bacteroides fragilis bft-2 gene for metalloprotease, complete cds 1,546 bp linear DNA AB026626.1
GI: 4757386
932. Bacteroides fragilis bft-1 gene for metalloprotease, complete cds 1,546 bp linear DNA AB026625.1
GI: 4757384
933. Bacteroides fragilis bft-3 gene for metalloprotease, complete cds 1,547 bp linear DNA AB026624.1
GI: 4757372
934. Clostridium histolyticum gene for class1 collagenase, complete cds 3,967 bp linear DNA AB026889.1
GI: 6174835
935. Sequence 6 from U.S. Pat. No. 5,843,753 1,899 bp linear DNA AR062771.1 GI: 5990462
936. Sequence 5 from U.S. Pat. No. 5,843,753 2,052 bp linear DNA AR062770.1 GI: 5990461
937. Sequence 6 from U.S. Pat. No. 5,807,729 1,899 bp linear DNA AR039034.1 GI: 5958397
938. Sequence 5 from U.S. Pat. No. 5,807,729 2,052 bp linear DNA AR039033.1 GI: 5958396
939. Sequence 4 from U.S. Pat. No. 5,861,280 747 bp linear DNAAR030553.1 GI: 5943767
940. Sequence 1 from U.S. Pat. No. 5,861,280 2,052 bp linear DNA AR030552.1 GI: 5943766
941. M.musculus DNA for collagenase promoter 692 bp linear DNA X82000.1 GI: 854277
942. Sequence 3 from U.S. Pat. No. 5,747,322 866 bp linear DNA AR005002.1 GI: 3965881
943. Sequence 2 from U.S. Pat. No. 5,747,322 821 bp linear DNA AR005001.1 GI: 3965880
944. Sequence 1 from U.S. Pat. No. 5,747,322 734 bp linear DNA AR005000.1 GI: 3965879
945. Human type IV collagenase gene, promoter sequence 1,716 bp linear DNA U96098.1 GI: 2459743
946. Pseudomonas sp. ‘TAC II 18’ PAPRA gene 1,814 bp linear DNA Y17314.1 GI: 3169101
947. Pseudomonas fluorescens gene for metalloprotease, complete cds 1,431 bp linear DNA AB013895.1
GI: 3135219
948. Homo sapiens neutrophil collagenase (CLGNA) gene, promoter region and 5′UTR 968 bp linear DNA
AF059679.1 GI: 3075514
949. Sequence 6 from U.S. Pat. No. 5,691,162 1,899 bp linear DNA I76290.1 GI: 3012444
950. Sequence 5 from U.S. Pat. No. 5,691,162 2,052 bp linear DNA I76289.1 GI: 3012443
951. Streptococcus sanguis iga gene, strain SK85 5,741 bp linear DNA Y13461.1 GI: 2288964
952. Streptococcus sanguis iga gene, strain SK49 5,681 bp linear DNA Y13460.1 GI: 2288962
953. Streptococcus sanguis iga gene, strain SK4 5,740 bp linear DNA Y13459.1 GI: 2288960
954. Streptococcus sanguis iga gene, strain SK162 5,868 bp linear DNA Y13458.1 GI: 228895
955. Streptococcus sanguis iga gene, strain SK161 5,678 bp linear DNA Y13457.1 GI: 2288956
956. Streptococcus sanguis iga gene, strain SK115 5,681 bp linear DNA Y13456.1 GI: 2288954
957. Streptococcus sanguis iga gene, strain SK112 5,681 bp linear DNA Y13455.1 GI: 2288952
958. Streptococcus oralis iga gene 5,529 bp linear DNA Y13224.1 GI: 2288928
959. Homo sapiens collagenase-1 (MMP-1) promoter sequence 4,438 bp linear DNA AF023338.1
GI: 2564677
960. Porphyromonas gingivalis DNA for collagenase, complete cds 1,306 bp linear DNA AB006973.1
GI: 2505966
961. Sequence 9 from Patent EP0677586 3,979 bp linear DNA A46338.1 GI: 2300554
962. Sequence 1 from Patent EP0677586 487 bp linear DNA A46330.1 GI: 2300549
963. Clostridium perfringens DNA for lambda toxin (metalloprotease), complete cds 2,202 bp linear DNA
D45904.1 GI: 1345370
964. Serratia marcescens metalloprotease transporter genes, complete cds 3,145 bp linear DNA D83582.1
GI: 1208446
965. Clostridium perfringens DNA for large-conductive mechanosensitive channel homologue, complete cds
720 bp linear DNA D50309.1 GI: 786167
966. H.sapiens COL 10Al gene for collagen (alpha-1 type X) 3,226 bp linear DNA X60382.1 GI: 30094
967. B.thermoproteolyticus npr gene for thermolysin 2,033 bp linear DNA X76986.1 GI: 441266
968. S.hyicus gene for neutral metalloprotease 1,814 bp linear DNA X73315.1 GI: 312428
969. V.proteolytica gene for aminopeptidase 787 bp linear DNA X62698.1 GI: 297182
970. V.alginolyticus gene for collagenase 3,927 bp linear DNA X62635.1 GI: 48325
971. Serratia marcescens metalloprotease gene 3,871 bp linear DNA X55521.1 GI: 47238
972. Sequence 25 from U.S. Pat. No. 5,453,371 2,442 bp linear DNA 114775.1 GI: 1249684
973. Sequence 24 from U.S. Pat. No. 5,453,371 2,217 bp linear DNA 114774.1 GI: 1249683
974. Sequence 2 from U.S. Pat. No. 5,453,371 4,054 bp linear DNA 114754.1 GI: 1249663
975. Sequence 1 from U.S. Pat. No. 4,772,557 1,970 bp linear DNA 101070.1 GI: 313929

### Generating Engineered S. Typhimurium

S. Typhimuium can engineered to express an exogenous collagenase encoding polynucleotide and/or secondary active agent and/or cargo using routine recombinant techniques, which are described in one or more of the documents cited herein and/or will be appreciated by those of ordinary skill in the art. The exogenous collagenase encoding polynucleotides and/or secondary active agents and/or cargo can be included on a vector or vectors, and be transiently or stably incorporated into the S. Typhimuium using conventional recombinant engineering techniques. The exogenous collagenase encoding polynucleotide and/or secondary agents and/or cargo(s) can be operatively coupled to one or more regulatory elements, such as promoters, operons, or other elements (e.g., enhancers and/or the like) needed for expression or integration into the bacterium. The encoding polynucleotides can be codon optimized for expression in the S. Typhimurium.

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

In some embodiments, the exogenous collagenase encoding polynucleotide is codon optimized for expression in the S. Typhimurium. Such codon optimized sequences are within the ambit of the ordinary skilled artisan in view of the description herein. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available.

In some embodiments, the S. Typhimurium are engineered to at least express an exogenous collagenase using a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the exogenous collagenase and/or secondary active agent and/or cargo described herein can be delivered directly to a host cell, e.g., an S. Typhimurium, and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the exogenous collagenase and/or secondary active agent and/or cargo described herein of the present disclosure. In some embodiments, the naked polynucleotide can contain other nucleic acids and/or polynucleotides in addition to the exogenous collagenase and/or secondary active agent and/or cargo described herein of the present disclosure.

### Exemplary Regulatory Elements

In some embodiments, the polynucleotides and/or vectors thereof described herein can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530) (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE: CMV enhancers: the R-US' segment in LTR of HTLV-I (Mol. Cell. Biol., Vol. 8(1), p. 466-472, 1988): SV40 enhancer; and the intron sequence between exons 2 and 3 of rabbit β-globin (Proc. Natl. Acad. Sci. USA., Vol. 78(3), p. 1527-31, 1981).

In some aspects, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some aspects, the vector can contain a minimal promoter. In some aspects, the minimal promoter is the Mecp2 promoter, tRNA promoter, or U6. In a further embodiment, the minimal promoter is tissue specific. In some aspects, the length of the vector polynucleotide the minimal promoters and polynucleotide sequences is less than 4.4 Kb.

To express a polynucleotide, the vector can include one or more transcriptional and/or translational initiation regulatory sequences, e.g. promoters, that direct the transcription of the gene and/or translation of the encoded protein in a cell. In some aspects, a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g. ITGAM, CD43 promoter, CD14 promoter, CD45 promoter, CD68 promoter), urogenital cell specific promoters (e.g., Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70) promoter, Hsp90) promoter, pDawn, XVE/OlexA, GVG, and pOp/LhGR.

### Pharmaceutical Formulations

Also described herein are pharmaceutical formulations that can contain an amount, effective amount, and/or least effective amount, and/or therapeutically effective amount of one or more engineered S. Typhimurium cells of the present description (which are also referred to as the primary active agent or ingredient elsewhere herein) and/or secondary active agent(s) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, an active ingredient (e.g., a primary or secondary active agent) is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, the active agent(s), e.g., compounds, molecules, compositions, vectors, vector systems, cells, or any combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a cancer. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

Where appropriate the pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agent(s), including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, genetic modifying agent and any combination thereof.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-a, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, bifeprunox, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g., ibuprofen, naproxen, ketoprofen, and nimesulide), COX-2 inhibitors (e.g., rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, abendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telvancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g., lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicyclic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g., camptothecin derivatives (e.g., topotecan and irinotecan)), epidermal growth factor receptor blockade family agents (e.g., cetuximab, gefitinib), farnesyltransferase inhibitors (e.g., L-778-123), COX-2 inhibitors (e.g., rofecoxib, celecoxib, and etoricoxib), bFGF and VEGF targeting agents (e.g., bevazucimab and thalidomide), NBTXR3, Nimoral, trans sodium crocetinate, NVX-108, and combinations thereof. See also e.g., Kvols, L. K., J Nucl Med 2005: 46:187S-190S.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more agents included in the pharmaceutical formulation can alone, or in combination, kill cancer cells, inhibit cancer growth, inhibit cancer metastasis, or have one or more other chemotherapeutic effects.

The effective amount, least effective amount, and/or therapeutically effective amount of the primary and an optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and an optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate: polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammator anti-histamines, anti-infectives, chemotherapeutics, and any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components, devices, containers, and/or the like that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof is in need of a cancer treatment.

### Methods of Using the Engineered S. Typhimurium

Described in certain example embodiments herein are methods of (a) treating and/or preventing a disease or a symptom thereof in a subject. (b) modifying a cell, tissue, organ, and/or tumor microenvironment of a subject, (c) modifying an extracellular matrix or component thereof optionally of a subject, (d) modifying a collagen matrix optionally of a subject; or (e) any combination of (a)-(d) the method including the step of administering an engineered S. Typhimurium bacterium, population thereof, and/or progeny thereof as described herein or a pharmaceutical formulation thereof to the subject, extracellular matrix or component thereof, collagen matrix, or combination thereof. In some embodiments, administration is to a tumor microenvironment. In some embodiments, a secondary agent is administered simultaneously or sequentially with the engineered S. Typhimurium, progeny thereof, or pharmaceutical formulation thereof.

In certain example embodiments, the disease is a cancer. In certain example embodiments, the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis fungoide, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.

In certain example embodiments, the method further comprises administering one or more secondary active agents to the subject. In certain example embodiments, the one or more secondary active agents comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, a genetic modifying agent, a vaccine, or any combination thereof.

In certain example embodiments, the engineered bacterium, population thereof, and/or progeny thereof as described herein, or the pharmaceutical formulation thereof is effective to treat a disease in the subject in need thereof.

Exemplary routes of administration are described herein and will be appreciated by those of ordinary skill in the art in view of the description provided herein.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

This Example can demonstrate modification of a strain of S. Typhimurium VNP20009 (ATCC 202165, American Type Culture Collection, Manassas, VA), which was tested in multiple clinical studies and shown safe for human administration to constitutively express a collagen-degrading metalloproteinase (collagenase) encoded on a plasmid [1], [2]. While S. Typhimurium VNP20009 has shown immense promise as a form of bacteria-based cancer therapy in vitro and in animal models, its success has not translated to the clinic [3]. This lack of efficacy may be due to insufficient tumor colonization, which was poor in clinical trials [1], [2]. A major reason for this may be the overexpression of collagen expressed in many tumors, which may be correlated with reduced bacterial intratumoral penetration and colonization [4]. Moreover, high collagen content impedes macromolecular and particle-based drug transport in tumors, which can be improved by administering collagenase [5]. Applicant hypothesized that a strain derived from S. Typhimurium VNP20009 that can actively degrade the collagen matrix holds the potential to improve bacterial tumor colonization and aid in the transport and improve the efficacy of co-administered chemotherapeutics. Herein, we comprehensively describe our experimental methods and results demonstrating the function of the engineered strain.

### Methods

### Cloning of a Bacterial Collagenase Gene and Plasmid Construction

The prtV gene encoding a metalloproteinase was cloned from Vibrio parahaemolyticus EB101 (ATCC 17802) using the Lambda-PCR technique recently developed by the Senger Laboratory at Virginia Tech [6]. In this restriction enzyme-free technique, forward and reverse primers are designed to flank the gene of interest. A short sequence (approx. 18-25 bp) matching the sequence immediately upstream of the insertion site (5′ to 3′) was added to the 5′ end of the forward primer, while a sequence containing the reverse complement of the 18-25 bp immediately downstream of the insertion site was added to the 5′ end of the reverse primer. Completion of PCR for cloning the gene, therefore, resulted in a double-stranded (DS) “mega-primer” (i.e., fragments that contained the cloned gene along with short regions of the destination vector on the 5′ and 3′ ends). Prior to gene cloning, the reverse primer was phosphorylated using T4 polynucleotide kinase (Thermo Fisher Scientific, Waltham, MA). PCR was performed using the high-fidelity DNA polymerase Phusion (Thermo Fisher). The minus strand of purified DS mega-primer product was digested using Lambda exonuclease (Thermo Fisher) to produce a single-stranded (SS) mega-primer. The pBAD LIC cloning vector (8A) was a gift from Scott Gradia (Addgene plasmid #37501: http://n2t.net/addgene:37501: RRID:Addgene_37501), and was used as the destination vector for this work. The gene insertion reaction is an Omega-PCR reaction, similar to the description in [7]. Approximately 75 ng of the vector, 2.5 μL of the SS mega-primer, and a reverse primer designed to bind to the plasmid vector in a distal region from the gene insertion site (final concentration of 2 μM) were combined in a 25 μL Phusion PCR reaction. The resulting Omega-PCR product was then digested using DpnI (Fisher Scientific, Pittsburgh, PA) for approximately 16 hr at 37° C., purified, and used to transform E. coli NEB 10-beta (New England BioLabs, Ipswich, MA) via standard heat shock methods.

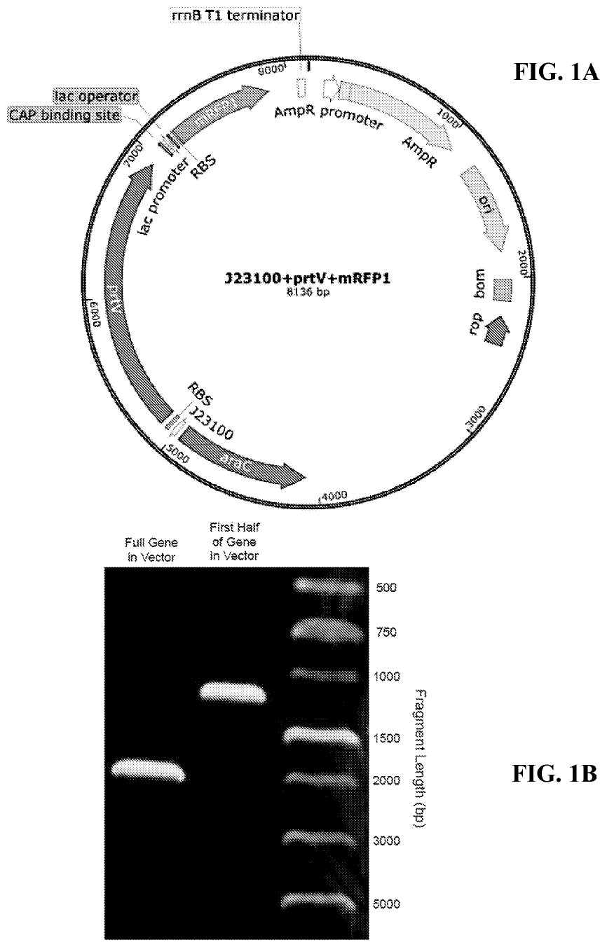

Applicant replaced the araBAD promoter natively present in the plasmid vector with the constitutive BioBrick promoter BBa_J23100 via standard site-directed mutagenesis to facilitate expression without the need for a chemical inducer (i.e., arabinose). Additionally, the BioBrick part BBa_J04550 (excluding the BBa_B0015 terminator) was cloned and inserted downstream of prtV but still within the multiple-cloning site (MCS) via Lambda-PCR. The final plasmid thus encoded constitutive expression of both prtV and mRFP1 (FIG. 1A-1B).

A colony containing the correct size insert was cultured overnight, and the DNA construct was extracted from E. coli and used to transform the restriction-deficient strain of S. Typhimurium JR501 [8]. Finally, DNA from this strain was extracted and used to transform VNP20009 as well as its parental strain, 14028. Hereafter, these strains are referred to as S. Typhimurium VNP20009prtV and 14028prtV.

A plasmid to encode constitutive mRFP1 expression was also constructed. For the preliminary work detailed herein, the full BBa_J04450 sequence was cloned into pBAD LIC (8A) via Lambda-PCR at a site outside the MCS (thus, the MCS did not contain a coding sequence). Unless otherwise noted, we do not give additional nomenclature to S. Typhimurium VNP20009 expressing this plasmid; all VNP20009 in control experiments expressed this plasmid.

### Microfluidic Experiments

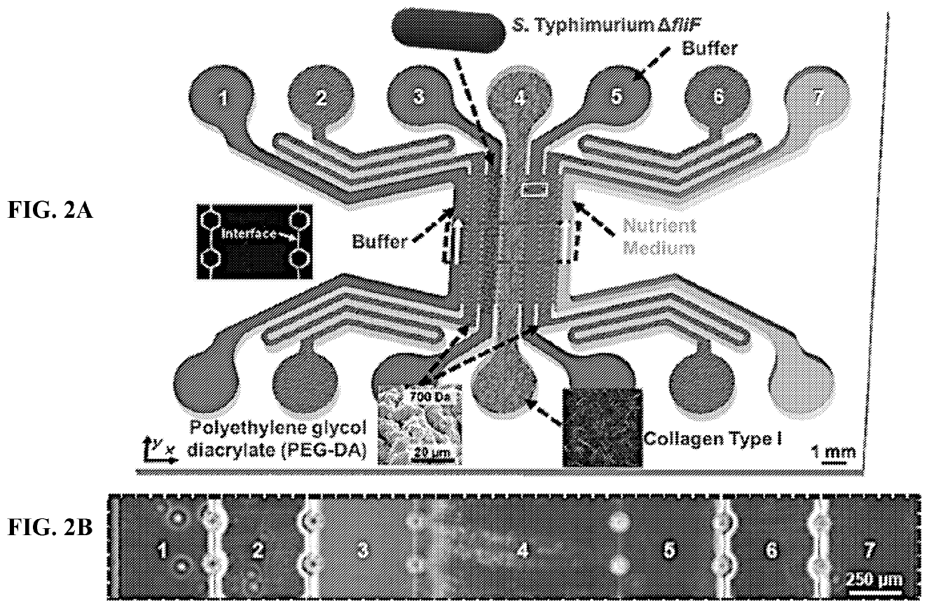

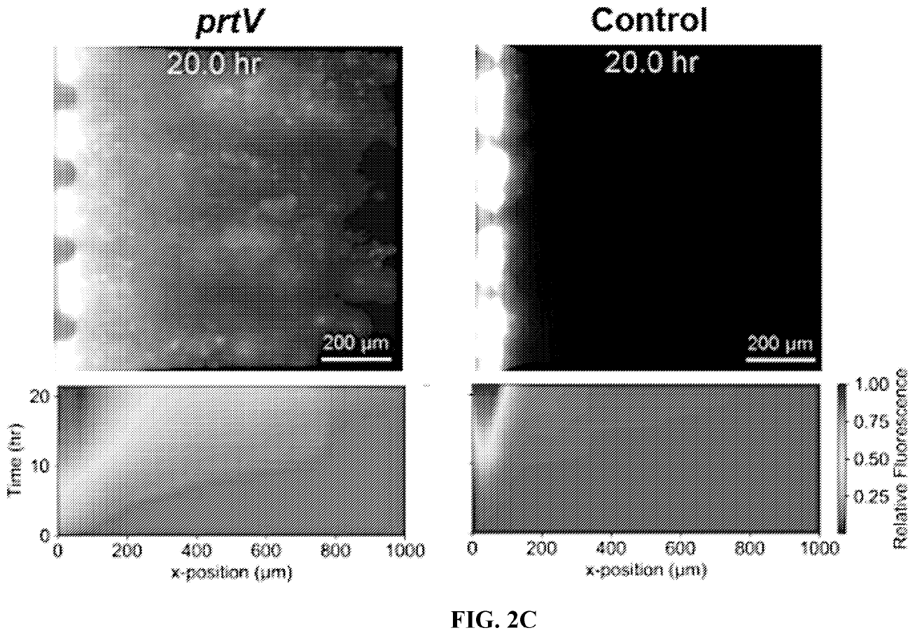

In order to evaluate the transport properties of the S. Typhimurium VNP20009prtV, Applicant performed experiments in a custom microfluidic platform containing a collagen barrier separating channels containing bacteria and buffer medium (FIG. 2A-2C). This device delivers a constant supply of nutrients to support bacterial growth and protein expression under quasi-static conditions. For all experiments, a relatively dense collagen matrix of 5 mg/mL was used. In order to decouple the confounding effects of differences in swimming motility and transport, Applicant used non-motile ΔfliF strains, which were transported by small interstitial flow across the collagen barrier. Initially, the bacteria were seeded only in the side of the hydrogel. Prior to experiments, the bacteria were culture overnight at 37° C., 100 RPM in lysogeny broth (LB: 1% tryptone, 0.5% yeast extracts, 1% NaCl) supplemented with 100 μg/mL ampicillin. Prior to experiments, bacteria were diluted 100× in fresh LB and grown to an OD600 of 1.0. The bacteria were then diluted 10× in PBS and pipetted gently into one channel adjacent to the collagen and a dilute solution of bacterial growth medium (LB) was continuously flowed through the outermost channel farthest from the bacteria introduction channel, while buffer solution (PBS) was flowed through the closer outermost channel.

### Results

Applicant has cloned the prtV gene from V. parahaemolyticus using the Lambda-PCR technique and performed several simple assays to validate and evaluate its expression in S. Typhimurium. These experiments demonstrate that the enzyme is expressed constitutively, secreted, and exhibits enzymatic activity against gelatin and collagen type I. After validating the successful engineering of prtV-expressing strains, Applicant performed experiments wherein the bacteria were allowed to grow and colonize collagen gel contained inside microfluidic devices. The penetration distance of S. Typhimurium VNP20009prtV was significantly enhanced relative to the parental control strain.

Cloning and Constitutive Expression of prtV in S. Typhimurium VNP 20009

While a number of collagenases have been recombinantly expressed in heterologous Gram-negative hosts, they usually accumulate intracellularly, thus rendering them ineffective against extracellular ECM absent of bacterial cell lysis. Applicant selected the PrtV collagenase form Vibrio parahaemolyticus for cloning and recombinant expression in S. Typhimurium VNP20009 based on prior findings that the enzyme is actively secreted when expressed by E. coli [6], [9]. To facilitate robust expression without significant detriment to bacterial fitness, we designed a constitutive expression circuit in a medium-copy number vector (FIG. 1A). To clone the gene, we adopted the recently developed Lambda-PCR technique from the Senger Laboratory at Virginia Tech. This method employs the Omega-PCR method coupled with a Lambda exonuclease digestion step for restriction enzyme- and ligation-free cloning [7]. In using this method, Applicant found that cloning DNA in fragments smaller than approximately 1 kbp significantly increased the success rate: thus the 1835 bp prtV gene was cloned in two fragments and confirmed via gel electrophoresis screening of colony-PCR products (FIG. 1B).

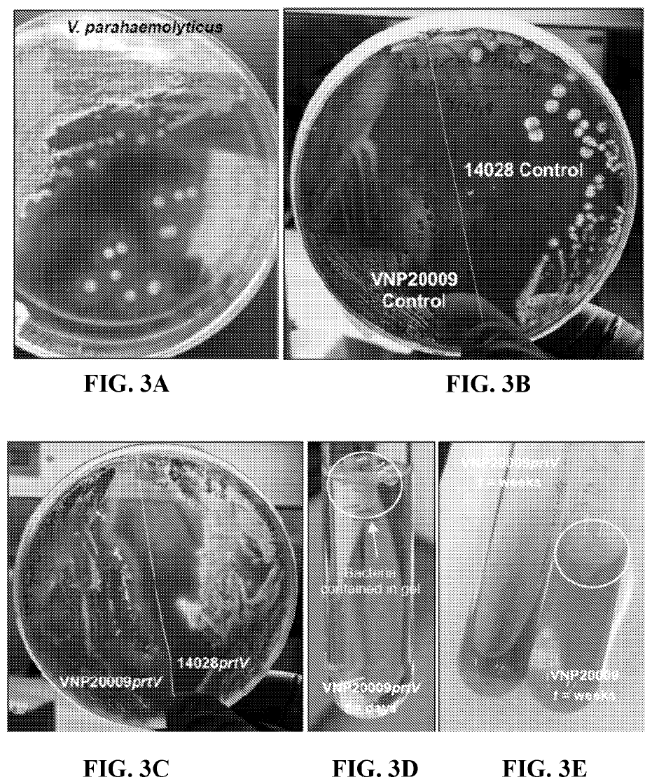

Following cloning, we first confirmed expression, secretion, and proteolytic activity of PrtV using a simple gelatin-based assay [10]. In this assay, proteolytic activity is directly observable via the clouding of nutrient agar supplemented with gelatin. Collagen is one of the primary constituents of gelatin. After approximately 40 hr incubation at 37° ° C., turbid zones appeared surrounding colonies V. parahaemolyticus (FIG. 3A) but not around colonies of wild-type S. Typhimurium (FIG. 3B). However, turbid zones were observable on plates containing S. Typhimurium harboring prtV plasmids (FIG. 3C). This confirmed that active PrtV was being secreted by the bacteria. We further confirmed expression by inoculating a 10% gelatin semi-solid medium with our VNP20009prtV strain (FIG. 3D) and wild-type VNP20009. After several weeks' incubation at room temperature, VNP20009prtV had completely liquefied the gelatin, while the control strain remained constrained by semi-solid gelatin near the inoculation site (FIG. 3E).

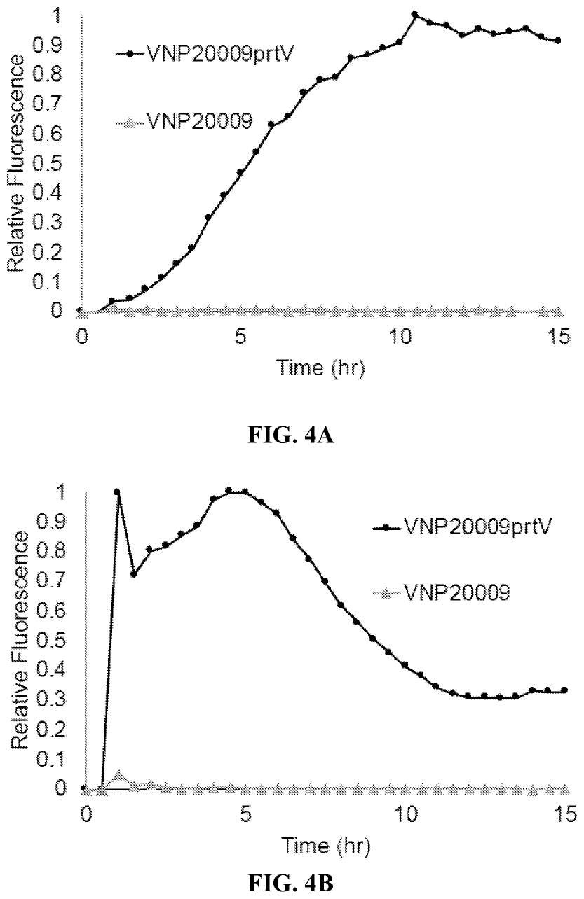

While gelatin-based assays confirmed the expression and secretion of PrtV, a more definitive assay for the bacteria's ability to degrade relevant tumor ECM constituents was needed to validate their potential for enhanced transport in and colonization of tumors. Collagen type I is one of the primary stromal components in many tumors. Applicant quantitatively measured collagen degradation by prtV and control strains using dye-quenched collagen type I, which releases fluorescent fluorescein upon protein cleavage. As expected, a strong increase in fluorescein fluorescence was detected in samples containing prtV strains but not in samples containing control strains (FIG. 4A). Nevertheless, a large amount of bacterial growth was detected in all cases via RFP fluorescence (FIG. 4B).

### Transport Experiment Results

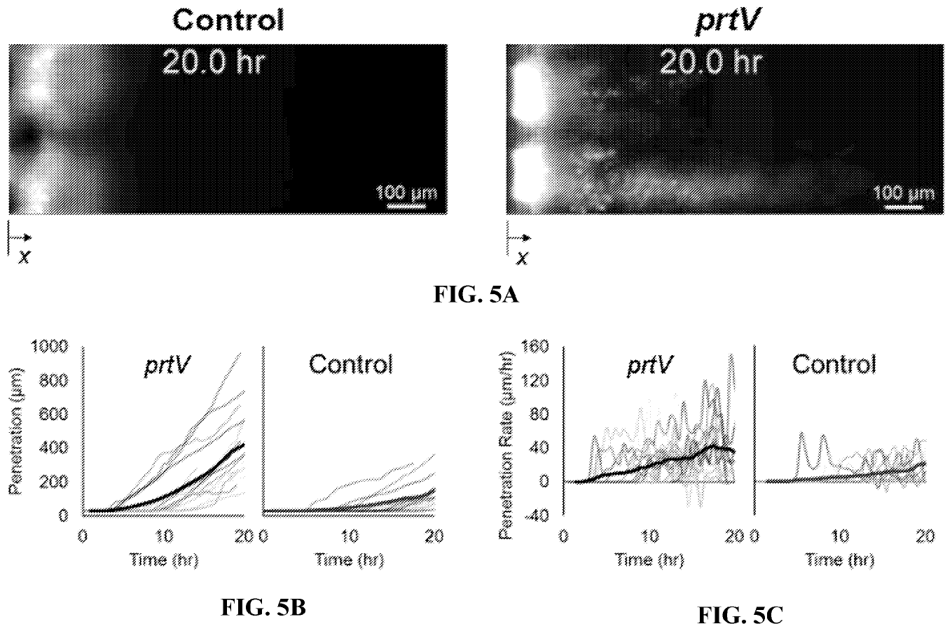

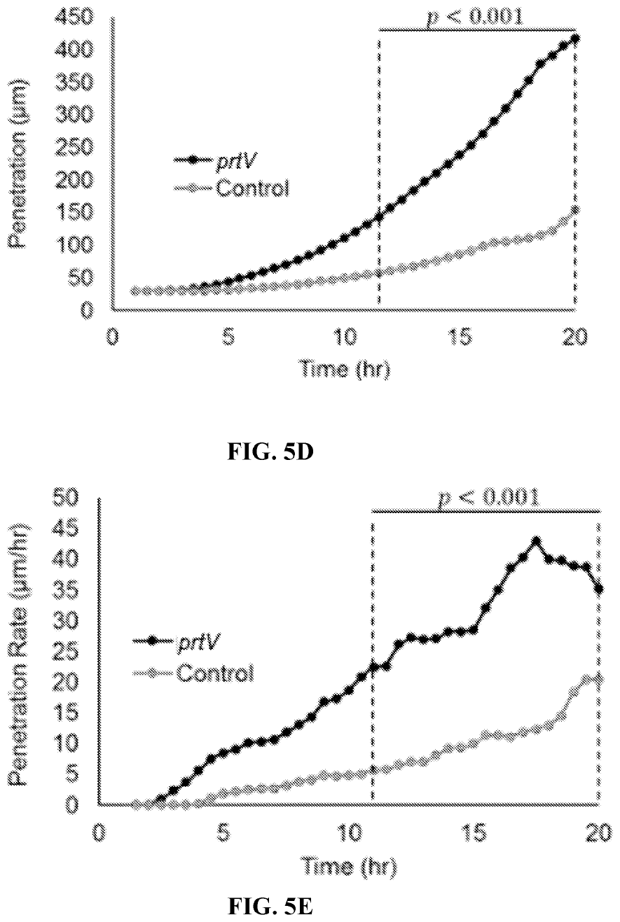

Applicant quantified the bacterial penetration via time-lapse fluorescent microscopy (FIG. 5A). Over a period of 20 hr, the collagenase-secreting S. Typhimurium VNP20009prtV were transported deeper into collagen gel on average, with penetration and penetration rate being significantly greater than that of the parental control strain for times greater than 11.5 hr and 11.0 hr, respectively (FIG. 5B-5E). These results indicate the functional performance of the engineered bacteria, with implications for enhanced transport in and colonization of tumors.

### REFERENCES FOR EXAMPLE 1

[1] J. F. Toso et al., “Phase I Study of the Intravenous Administration of Attenuated Salmonella typhimurium to Patients With Metastatic Melanoma,” J. Clin. Oncol., vol. 20, no. 1, pp. 142-152, 2002.
[2] J. Nemunaitis et al., “Pilot trial of genetically modified, attenuated Salmonella expressing the E. coli cytosine deaminase gene in refractory cancer patients.,” Cancer Gene Ther., vol. 10, pp. 737-744, 2003, doi: 10.1038/sj.cgt. 7700634.
[3] S. Zhou, C. Gravekamp, D. Bermudes, and K. Liu, “Tumour-targeting bacteria engineered to fight cancer,” Nat. Rev. Cancer, vol. 18, no. 12, pp. 727-743, 2018, doi: 10.1038/s41568-018-0070-z.
[4] S. B. Suh et al., “Nanoscale Bacteria-Enabled Autonomous Drug Delivery System (NanoBEADS) Enhances Intratumoral Transport of Nanomedicine,” Adv. Sci., vol. 6, no. 3, 2019, doi: 10.1002/advs.201801309.
[5] A. Dolor and F. C. Szoka, “Digesting a Path Forward: The Utility of Collagenase Tumor Treatment for Improved Drug Delivery,” Mol. Pharm., vol. 15, no. 6, pp. 2069-2083, 2018, doi: 10.1021/acs.molpharmaceut.8b00319.
[6] C. Y. Lee, S. C. Su, and R. B. Liaw, “Molecular analysis of an extracellular protease gene from Vibrio parahaemolyticus,” Microbiology, vol. 141, no. 10, pp. 2569-2576, 1995, doi: 10.1099/13500872-141-10-2569.
[7] L. Chen, F. Wang, X. Wang, and Y. G. Liu, “Robust one-tube Ω-PCR strategy accelerates precise sequence modification of plasmids for functional genomics,” Plant Cell Physiol., vol. 54, no. 4, pp. 634-642, 2013, doi: 10.1093/pcp/pct009.
[8] S. P. Tsai, R. J. Hartin, and J.-I. Ryu, “Transformation in Restriction-deficient Salmonella typhimurium LT2,” Microbiology, vol. 135, no. 9, pp. 2561-2567, 1989, doi: 10.1099/00221287-135-9-2561.
[9] M.-S. Yu and C.-Y. Lee, “Expression and characterization of the prtV gene encoding a collagenase from Vibrio parahaemolyticus in Escherichia coli,” Microbiology, vol. 145, no. 1, pp. 143-150, 1999, doi: 10.1099/13500872-145-1-143.
[10] J. Smith, Harry L. and K. Goodner, “Detection of Bacterial Gelatinases by Gelatin-agar Plate Methods,” J. Bacteriol., vol. 76, no. 6, pp. 662-665, 1958.

### Example 2

Chemotherapeutics often have limited efficacy and high systemic toxicity. Bacteria could serve as autonomous drug carrier with immunotherapeutic potential. This Example describes and demonstrates at least the generation of exemplary engineered S. Typhimurium VNP 20009 that secretes a heterologous collagenase without debilitation effects on fitness of the engineered bacteria. This Example also at least evaluates the effect of collagenase secretion on the bacterial interstitial transport.

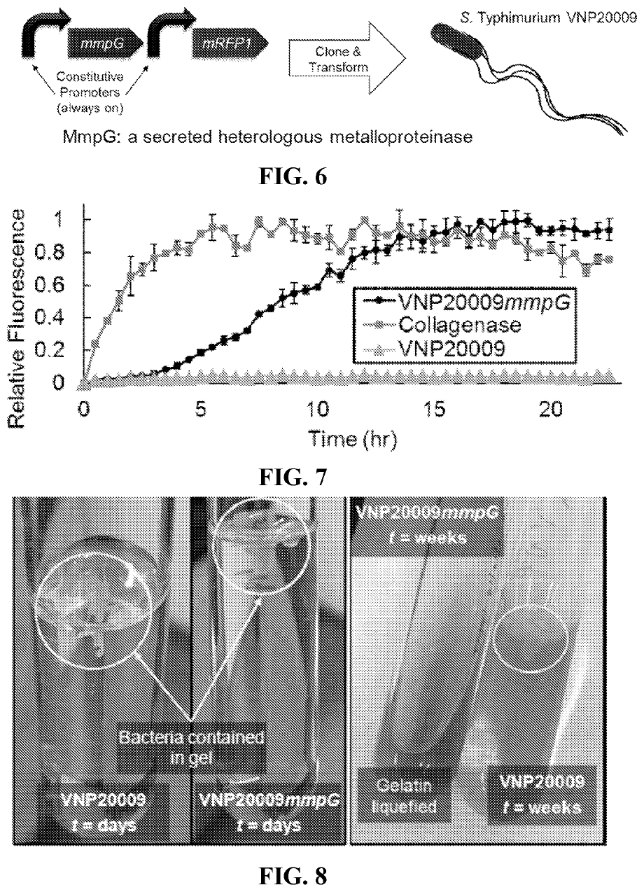

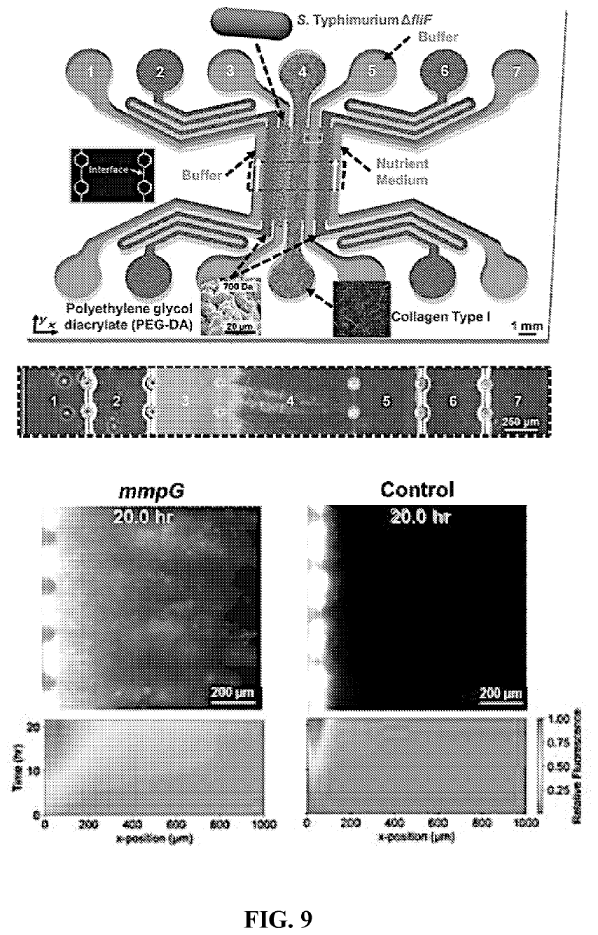

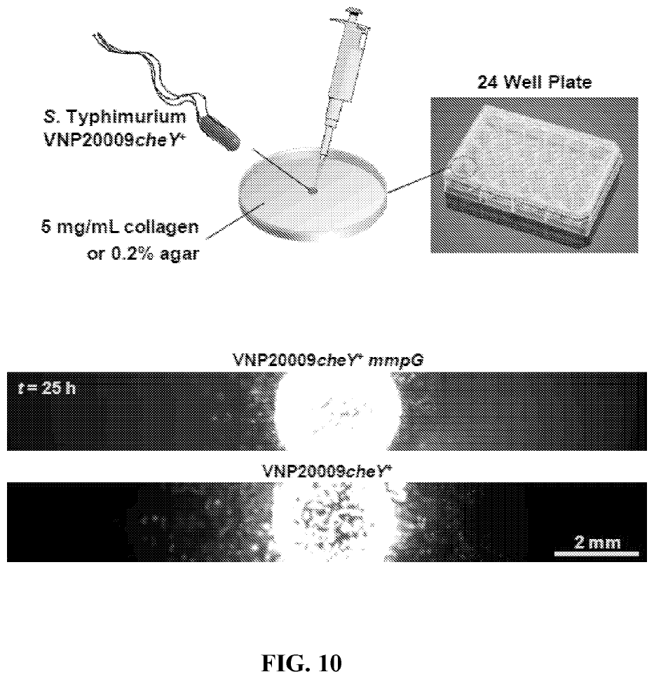

Without being bound by theory, Applicant hypothesized that engineering bacteria, and more particularly tumor-targeting bacteria, to express collagenase in situ can enhance bacterial tumor penetration. FIG. 6 shows an exemplary scheme for engineering a collagenase secreting Salmonella. FIGS. 8-10 show various assays, including microfluidic assays, to evaluate characteristics of the engineered bacteria, such as dye-quenched collagen type I assay, microfluidic assays of advective transport of non-motile bacteria under oscillatory interstitial flow, and swim plate assays of motile bacteria transport.

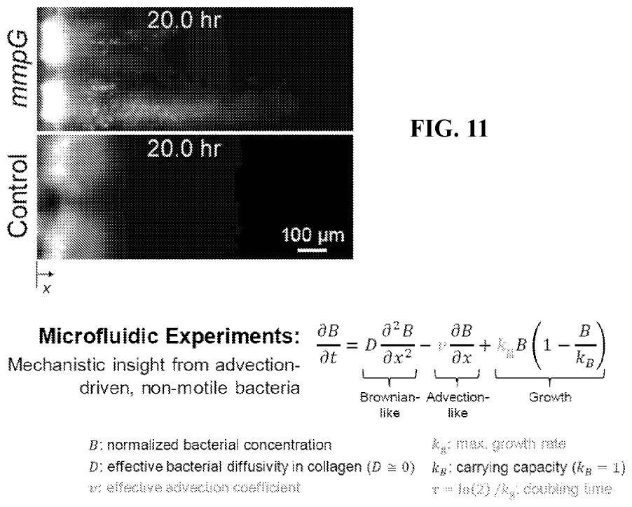

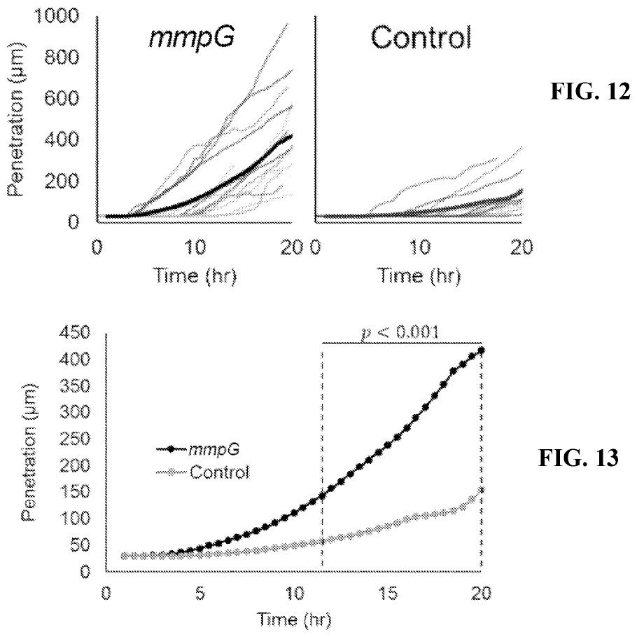

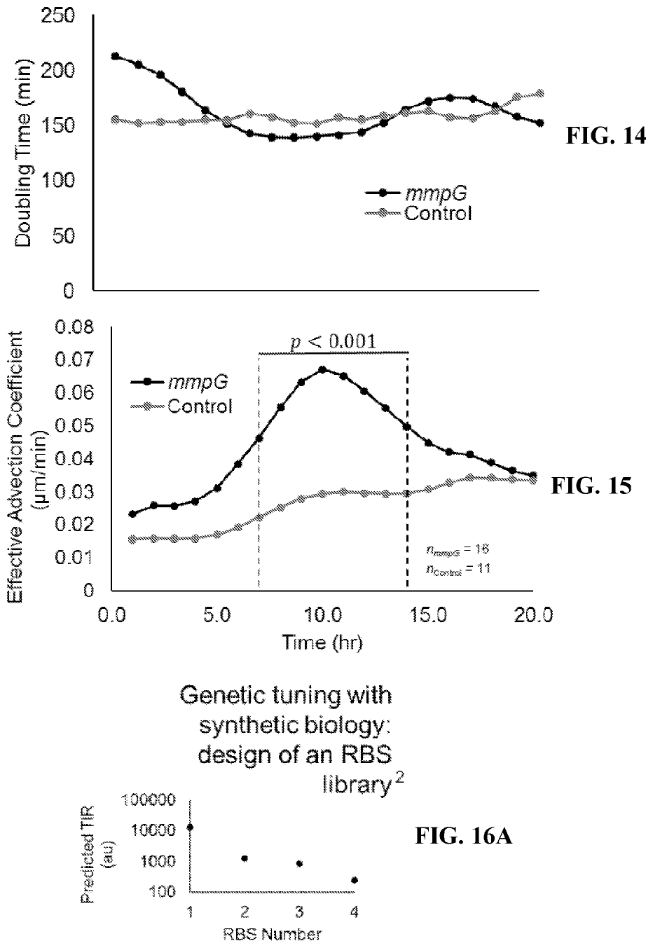

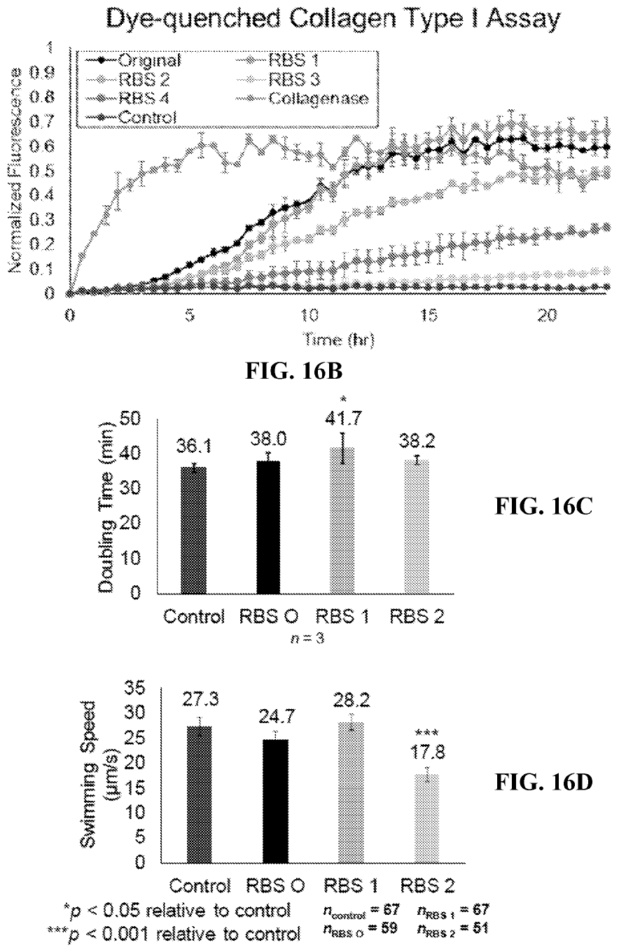

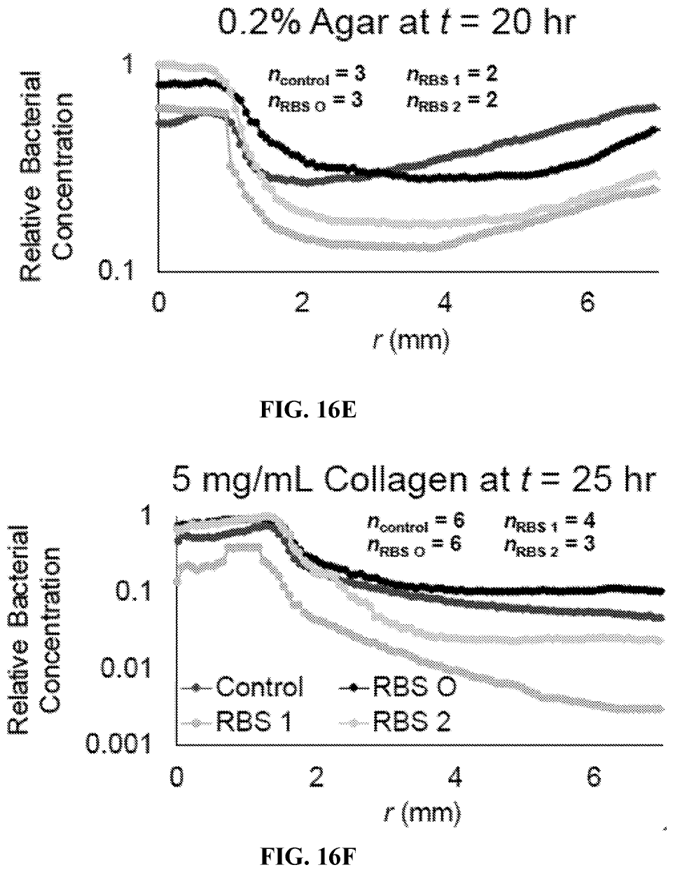

FIGS. 11-16F shows microfluidic experimental results and genetic tuning of motile bacteria from control and engineered VNP2009mmpG bacteria.

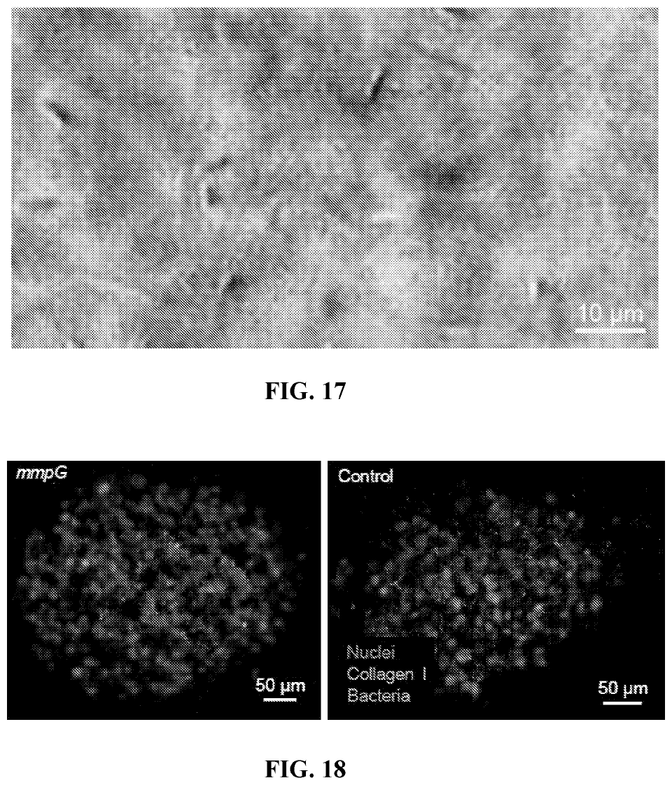

FIG. 17 shows a microscopic image bacteria swimming in about 5 mg/mL collagen. FIG. 18 shows fluorescent microscopic images showing bacteria infiltration into pancreatic tumor organoids.

Results at least demonstrated that collagenase secretion enhances the penetration of non-motile S. Typhimurium by about 171% under low velocity oscillatory flow mimicking interstitial flow in the tumor microenvironment. Further, balancing collagenase secretion rate and its deleterious effects on motility and growth enhances the transport and distal colonization of motile strains. Moreover, localized collagenase secretion has the potential to augment bacteria-based cancer therapy.

### REFERENCES FOR EXAMPLE 2

1. Suh et al. (2019) Adv. Sci. 6(3).
2. RBSs design using the RBS Calculator v2.0 Borujeni et al., (2014). Nuc. Acids Res. 42(4).
3. Drifka et al. (2013). Lab Chip. 13(19).
4. Leaman et al. (2018). ACS Synth. Biol. 7(4).

### ***

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An engineered Salmonella Typhimurium (S. Typhimurium) bacterium, population thereof, and/or progeny thereof, the engineered S. Typhimurium bacterium comprising: an exogenous collagenase encoding polynucleotide, polypeptide product thereof, or both, wherein the engineered S. Typhimurium strain is an S. Typhimurium14028 or S. Typhimurium VNP20009.
2. The engineered bacterium, population thereof, and/or progeny thereof of aspect 1, wherein the collagenase encoding polynucleotide is or encodes a collagenase or functional domain thereof as set forth in Table 1.
3. The engineered bacterium, population thereof, and/or progeny thereof of any one of aspects 1-2, wherein the exogenous collagenase gene is a metalloproteinase gene.
4. The engineered bacterium, population thereof, and/or progeny thereof of any one of aspects 1-3, wherein the collagenase gene is prtV from Vibrio parahaemolyticus EB101, a homologue thereof, an orthologue thereof, or a paralogue thereof.
5. The engineered bacterium, population thereof, and/or progeny thereof of any one of aspects 1-4, wherein the exogenous collagenase encoding polynucleotide is present on a plasmid, cosmid, or artificial chromosome.
6. The engineered bacterium, population thereof, and/or progeny thereof of any one of aspects 1-5, wherein the exogenous collagenase encoding polynucleotide is operably coupled to one or more regulatory elements, optionally wherein the one or more regulatory elements is or comprises a promoter, wherein the promoter is a constitutive promoter, inducible promoter, tissue or tumor specific promoter, or any permissible combination thereof.
7. The engineered bacterium, population thereof, and/or progeny thereof of any one of aspects 1-6, wherein the exogenous collagenase encoding polynucleotide is constitutively expressed, is inducibly expressed, or is selectively expressed by the engineered bacterium.
8. The engineered bacterium, population thereof, and/or progeny thereof of any one of aspects 1-7, wherein the engineered bacterium, population thereof, and/or progeny thereof has increased tumor or tumor microenvironment penetration, increased tumor microenvironment retention, increased tumor colonization, or any combination thereof as compared to a parent S. Typhimurium, optionally of the strain S. Typhimurium 14028 or strain S. Typhimurium VNP20009.
9. The engineered bacterium, population thereof, and/or progeny thereof of any one of aspects 1-8, wherein the engineered bacterium, population thereof, and/or progeny thereof is capable of degrading a collagen matrix.
10. The engineered bacterium, population thereof, and/or progeny thereof of any one of aspects 1-9, wherein the engineered bacterium, population thereof, and/or progeny thereof is capable of producing and/or secreting a collagenase polypeptide and/or functional domain thereof.
11. The engineered bacterium, population thereof, and/or progeny thereof of any one of aspects 1-10, further comprising a second active agent, a cargo, or both, wherein the second active agent, cargo, or both is/are coupled to, integrated with, contained within, or otherwise associated with the engineered bacterium, population thereof, and/or progeny thereof.
12. The engineered bacterium, population thereof, and/or progeny thereof of any one of aspects 1-11, wherein collagen matrix penetration, tumor microenvironment penetration, and/or extracellular matrix penetration is increased 10-1,000 percent or more as compared to a S. Typhimurium parent bacterium, optionally of the strain S. Typhimurium 14028 or strain S. Typhimurium VNP20009.
13. A pharmaceutical formulation comprising: an engineered bacterium, population thereof, and/or progeny thereof of any one of aspects 1-12; and a pharmaceutically acceptable carrier.
14. The pharmaceutical formulation of aspect 13, further comprising one or more secondary active agents.
15. The pharmaceutical formulation of aspect 13, wherein the one or more secondary active agents is/are or comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, a genetic modifying agent, a vaccine, or a combination thereof.
16. A method of (a) treating and/or preventing a disease or a symptom thereof in a subject, (b) modifying a cell, tissue, organ, and/or tumor microenvironment of a subject, (c) modifying an extracellular matrix or component thereof optionally of a subject, (d) modifying a collagen matrix optionally of a subject; or (e) any combination of (a)-(d) the method comprising: administering an engineered bacterium, population thereof, and/or progeny thereof of any one of aspects 1-12 or a pharmaceutical formulation thereof as in any one of claims 13-15 to the subject, extracellular matrix or component thereof, collagen matrix, or combination thereof.
17. The method of aspect 16, wherein the disease is a cancer.
18. The method of aspect 17, wherein the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis fungoide, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.
19. The method of any one of aspects 16-18, further comprising administering one or more secondary active agents to the subject.
20. The method of aspect 19, wherein the one or more secondary active agents comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoireanti-histamines, anti-infectives, radiation sensitizers, chemotherapeutics, a genetic modifying agent, a vaccine, or any combination thereof.
21. The method of any one of aspects 16-20, wherein the engineered bacterium, population thereof, and/or progeny thereof of any one of aspects 1-12 or the pharmaceutical formulation thereof as in any one of aspects 13-15 is effective to treat a disease in the subject in need thereof.
22. A kit for treating and/or preventing a disease in a subject in need thereof comprising: an engineered bacterium, population thereof, and/or progeny thereof of any one of aspects 1-12 or a pharmaceutical formulation thereof as in any one of aspects 13-15, optionally one or more secondary active agents, and/or optionally one or more delivery reagents and/or devices, one or more storage reagents and/or devices, one or more culture reagents and/or devices, or any combination thereof; and instructions in a tangible medium expression directing a user to administer the engineered bacterium, population thereof, and/or progeny thereof of any one of aspects 1-12 or a pharmaceutical formulation thereof as in any one of aspects 13-15, and optionally the one or more secondary active agents to the subject in need thereof.
23. The kit of aspect 22, wherein the subject in need thereof has a cancer.
24. The kit of aspect 23, wherein the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis fungoide, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.

What is claimed is:

1. An engineered Salmonella Typhimurium (S. Typhimurium) bacterium, population thereof, and/or progeny thereof, the engineered S. Typhimurium bacterium comprising:
an exogenous collagenase encoding polynucleotide, polypeptide product thereof, or both, wherein the engineered S. Typhimurium strain is an S. Typhimurium14028 or S. Typhimurium VNP20009.

2. The engineered bacterium, population thereof, and/or progeny thereof of claim 1, wherein the collagenase encoding polynucleotide is or encodes a collagenase or functional domain thereof as set forth in Table 1.

3. The engineered bacterium, population thereof, and/or progeny thereof of claim 1, wherein the exogenous collagenase gene is a metalloproteinase gene.

4. The engineered bacterium, population thereof, and/or progeny thereof of claim 1, wherein the collagenase gene is prtV from Vibrio parahaemolyticus EB101, a homologue thereof, an orthologue thereof, or a paralogue thereof.

5. The engineered bacterium, population thereof, and/or progeny thereof of claim 1, wherein the exogenous collagenase encoding polynucleotide is present on a plasmid, cosmid, or artificial chromosome.

6. The engineered bacterium, population thereof, and/or progeny thereof of claim 1, wherein the exogenous collagenase encoding polynucleotide is operably coupled to one or more regulatory elements, optionally wherein the one or more regulatory elements is or comprises a promoter, wherein the promoter is a constitutive promoter, inducible promoter, tissue or tumor specific promoter, or any permissible combination thereof.

7. The engineered bacterium, population thereof, and/or progeny thereof of claim 1, wherein the exogenous collagenase encoding polynucleotide is constitutively expressed, is inducibly expressed, or is selectively expressed by the engineered bacterium.

8. The engineered bacterium, population thereof, and/or progeny thereof of any one of claim 1, wherein the engineered bacterium, population thereof, and/or progeny thereof has increased tumor or tumor microenvironment penetration, increased tumor microenvironment retention, increased tumor colonization, or any combination thereof as compared to a parent S. Typhimurium, optionally of the strain S. Typhimurium 14028 or strain S. Typhimurium VNP20009.

9. The engineered bacterium, population thereof, and/or progeny thereof of claim 1, wherein the engineered bacterium, population thereof, and/or progeny thereof is capable of degrading a collagen matrix.

10. The engineered bacterium, population thereof, and/or progeny thereof of any one of claim 1, wherein the engineered bacterium, population thereof, and/or progeny thereof is capable of producing and/or secreting a collagenase polypeptide and/or functional domain thereof.

11. The engineered bacterium, population thereof, and/or progeny thereof of claim 1, further comprising a second active agent, a cargo, or both, wherein the second active agent, cargo, or both is/are coupled to, integrated with, contained within, or otherwise associated with the engineered bacterium, population thereof, and/or progeny thereof.

12. The engineered bacterium, population thereof, and/or progeny thereof of claim 1, wherein collagen matrix penetration, tumor microenvironment penetration, and/or extracellular matrix penetration is increased 10-1,000 percent or more as compared to a S. Typhimurium parent bacterium, optionally of the strain S. Typhimurium 14028 or strain S. Typhimurium VNP20009.

13. A pharmaceutical formulation comprising:
an engineered bacterium, population thereof, and/or progeny thereof of any one of claim 1-12; and
a pharmaceutically acceptable carrier.

14. The pharmaceutical formulation of claim 13, further comprising one or more secondary active agents.

15. The pharmaceutical formulation of claim 13, wherein the one or more secondary active agents is/are or comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, a genetic modifying agent, a vaccine, or any combination thereof.

16. A method of
a. treating and/or preventing a disease or a symptom thereof in a subject;
b. modifying a cell, tissue, organ, and/or tumor microenvironment of a subject;
c. modifying an extracellular matrix or component thereof optionally of a subject;
d. modifying a collagen matrix optionally of a subject; or
e. a combination thereof
the method comprising:
administering an engineered bacterium, population thereof, and/or progeny thereof of claim 1 or a pharmaceutical formulation thereof the subject, extracellular matrix or component thereof, collagen matrix, or any combination thereof.

17. The method of claim 16, wherein the disease is a cancer.

18. The method of claim 17, wherein the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis fungoide, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.

19. The method of claim 16, further comprising administering one or more secondary active agents to the subject.

20. The method of claim 19, wherein the one or more secondary active agents comprise DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, a genetic modifying agent, a vaccine, or any combination thereof.

21. The method of claim 16, wherein the engineered bacterium, population thereof, and/or progeny thereof of claim 1 or the pharmaceutical formulation is effective to treat a disease in the subject in need thereof.

22. A kit for treating and/or preventing a disease in a subject in need thereof comprising:
an engineered bacterium, population thereof, and/or progeny thereof of claim 1 or a pharmaceutical formulation thereof, optionally one or more secondary active agents, and/or optionally one or more delivery reagents and/or devices, one or more storage reagents and/or devices, one or more culture reagents and/or devices, or any combination thereof; and
instructions in a tangible medium expression directing a user to administer the engineered bacterium, population thereof, and/or progeny thereof of claim 1 or a pharmaceutical formulation thereof, and optionally the one or more secondary active agents to the subject in need thereof.

23. The kit of claim 22, wherein the subject in need thereof has a cancer.

24. The kit of claim 23, wherein the cancer is acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, an astrocytoma, atypical teratoid/Rhabdoid tumors, basal cell carcinoma of the skin, bile duct cancer, bladder cancer, a bone cancer, a brain tumor or cancer, a glioblastoma, breast cancer, a bronchial tumor, Burkitt lymphoma, carcinoid tumor, a cardiac tumor, a germ cell tumor, an embryonal tumor, cervical cancer, cholangiocarcinoma, a chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, an endometrial cancer, an ependymoma, esophageal cancer, esthesioneuroblastoma, an extracranial germ cell tumor, an extragonadal germ cell tumor, an eye cancer, fallopian tube cancer, a gallbladder cancer, a gastric cancer, a gastrointestinal carcinoid tumor, a gastrointestinal stromal tumor, a central nervous system germ cell tumor, an extracranial germ cell tumor, an extragonadal germ cell tumor, an ovarian germ cell tumor, testicular cancer, gestational trophoblastic disease, Hairy cell leukemia, a head and/or neck cancer, a hepatocellular (liver) cancer, Langerhans cell histiocytosis, Hodgkin lymphoma, hypopharyngeal cancer, an islet cell tumor, a pancreatic neuroendocrine tumor, a kidney cancer, a laryngeal cancer, leukemia, a lip cancer, an oral cancer, a lung cancer, lymphoma, melanoma, Merkel cell carcinoma, mesothelioma, metastatic squamous cell neck cancer, a midline tract carcinoma with or without NUT gene changes, a multiple endocrine neoplasia syndrome, multiple myeloma, a plasma cell neoplasm, a mycosis fungoide, a myelodysplastic syndrome, a myelodysplastic/myeloproliferative neoplasm, chronic myelogenous leukemia, a nasal cancer, a sinus cancer, non-Hodgkin lymphoma, a pancreatic cancer, a paraganglioma, a paranasal sinus cancer, a parathyroid cancer, a penile cancer, a pharyngeal cancer, a pheochromocytoma, a pituitary cancer, a peritoneal cancer, a prostate cancer, a rectal cancer, a Rhabdomyosarcoma, a salivary gland cancer, a uterine sarcoma, Sézary syndrome, a skin cancer, a small intestine cancer, a colon cancer, a soft tissue sarcoma, a T-cell lymphoma, a throat cancer, an oropharyngeal cancer, a nasopharyngeal cancer, a hypopharyngeal cancer, a thymoma, a thymic carcinoma, a thyroid cancer, a transitional cell cancer of the renal pelvis and ureter, a urethral cancer, a uterine cancer, a vaginal cancer, a cervical cancer, a vascular tumor and/or cancer, a vulvar cancer, Wilms Tumor, or any combination thereof.
